(19) United States
(12) Patent Application Publication
(10) Pub. No.: US 2019/0352390 A1
(43) Pub. Date: Nov. 21, 2019

(54) ANTIBODIES TO IL-37

(71) Applicants: Monash University, Clayton, Victoria (AU); Hudson Institute of Medical Research, Clayton, Victoria (AU)

(72) Inventors: Andrew Ellisdon, Clayton, Victoria (AU); James Whisstock, Clayton, Victoria (AU); Claudia Nold, Clayton, Victoria (AU); Marcel Nold, Clayton, Victoria (AU)

(21) Appl. No.: 16/471,431

(22) PCT Filed: Dec. 22, 2017
(86) PCT No.: PCT/AU2017/051443

§ 371 Date: Jun. 19, 2019

(30) Foreign Application Priority Data
Dec. 23, 2016 (AU) 2016905344

(51) Int. Cl. C07K 16/24; G01N 33/68

(52) U.S. Cl. CPC C07K 16/244; G01N 2333/54; C07K 2317/76; G01N 33/6869

(57) ABSTRACT

The invention relates to IL-37, to antibodies and related fragments thereof for binding to IL-37, to production of said antibodies and fragments and to use of said antibodies and fragments for detection and therapy of various conditions. For example, the invention relates to an antigen binding site that binds to or specifically binds to IL-37 and inhibits IL-37 activity.

### FIELD OF THE INVENTION

The invention relates to IL-37, to antibodies and related fragments thereof for binding to IL-37, to production of said antibodies and fragments and to use of said antibodies and fragments for detection and therapy of various conditions.

### ASSOCIATED APPLICATIONS

This application claims priority from Australian provisional application AU 2016905344, the contents of which are hereby incorporated by reference in their entirety.

### BACKGROUND OF THE INVENTION

Interleukin (IL)-37 (formerly known as interleukin-1 family member 7, or IL-1F7) is a member of the IL-1 family which consists of the pro-inflammatory ligands IL-1α, IL-1β, IL-18, IL-33, IL-36α, IL-36μ, IL-36γ, and the anti-inflammatory members IL-1 receptor antagonist (IL-1Ra), IL-36Ra, IL-37 and IL-38. IL-37 distinguishes itself from most other anti-inflammatory cytokines by exerting functions that counter the activity of a broad spectrum of inflammatory assaults, including pro-inflammatory cytokines such as IL-1β, tumor necrosis factor (TNF) and interferon (IFN)γ as well as Toll-like receptors (TLRs). Accordingly, expression of IL-37 in the human lung epithelial cell line A549 suppressed the production of pro-inflammatory cytokines such as IL-1α and IL-6. As a mouse homolog for IL-37 has yet to be identified, in vivo studies used mice transgenic for human IL-37. These mice were protected in a number of animal models of disease, including lipopolysaccharide (LPS)-induced shock, experimental colitis, ischemic liver damage and obesity-induced inflammation.

IL-37 is inducible by various pro-inflammatory stimuli such as IL-1β, IL-18, TNF, IFNγ or TLR agonists. Encoded by the IL1F7 gene, IL-37 is expressed in five splice variants, termed IL1F7a-e. IL-37b, the longest and only isoform that possesses 5 of the 6 exons, is by far the best-studied splice variant. Similar to other IL-1 family ligands such as IL-1β and IL-18, IL-37 is expressed as a precursor molecule and contains a caspase-1 cleavage site.

IL-37 inhibits inflammation via two distinct mechanisms. On the one hand, IL-37 acts intracellularly by translocating into the nucleus and inhibiting pro-inflammatory cytokines in a Smad3-dependent manner. On the other hand, IL-37 is released from cells and then inhibits inflammation by binding to its cell surface receptor complex, which comprises IL-18 receptor α (IL-18Rα) and IL-1 receptor 8 (IL-1R8, previously known as single immuno-globulin IL-1R-related molecule, SIGIRR).

There are circumstances where inhibition of an anti-inflammatory response would be beneficial, for example in treating cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides an antigen binding site that binds to or specifically binds to IL-37 and inhibits IL-37 activity.

Preferably, the antigen binding site comprises an antigen binding domain of an antibody, the antigen binding domain binds to or specifically binds to IL-37 and inhibits IL-37 activity.

The IL-37 activity that may be inhibited by any antigen binding site of the invention includes: IL-37 binding to IL-18 receptor α (IL-18Rα), IL-1 receptor 8 or a complex of which comprises IL-18 receptor α (IL-18Rα) and IL-1 receptor 8; IL-37 binding to or interacting with SMAD3; IL-37 binding to or interacting with DNA; IL-37 mediated cell signalling; an IL-37 mediated anti-inflammatory response in vitro or in vivo; IL-37 mediated reduction in cytokine production or secretion; and/or autocrine or paracrine IL-37 mediated reduction in cytokine production or secretion. Preferably, the cytokine is any one or more described herein, including sICAM-1, IFN-gamma, I-TAC, G-SCF, IL-16, IL-10, IL-13, TNF, 1-309, IL-2, IL-7, M-CSF, TIMP-1, IL-1alpha, MIP-1alpha, RANTES, MIG, IL-1Ra, sTREM-1, MCP-5, IP-10, MCP-1, IL-23, KC, IL-1beta, MIP-2, IL-17, IL-17F, IL-4, IL-5, IKL-33, IL-25, IL-21, IL-22, eotaxin and IL-6. More preferably, the cytokine is any one or more of M-CSF, TIMP-1, IL-1alpha, MIP-1alpha, RANTES, MIG, IL-1Ra, sTREM-1, MCP-5, IP-10, MCP-1, IL-23, KC, IL-1beta, MIP-2, IL-17, eotaxin and IL-6. Most preferably, the pro-inflammatory cytokine is IL-1β, IL-6 and/or TNF. Preferably, IL-37 mediated activity is determined using monocytes, macrophages, peripheral blood mononuclear cells (PBMCs), cancer cells or a cell derived therefrom. Preferably, the cancer cell is a lung epithelial cell such as A549, or monocytic leukemia cell such as THP-1.

In any embodiment of the invention, the antigen binding site of the invention binds to or specifically binds to human IL-37. It will be understood that the IL-37 that is bound by the antigen binding site includes any homolog, or sequence variant of IL-37. Further, the human IL-37 may be in monomeric or dimeric form. The monomeric form of IL-37 may include variants of IL-37 which have a reduced propensity to form dimers, including an IL-37 molecule having a mutation of D73K or Y85A, or any one or more of the mutations described in PCT/AU2016/050495. Still further, the human IL-37 bound by the antigen binding site of the invention may comprise, consist essentially of or consist of an amino acid sequence corresponding to the propeptide of IL-37. Alternatively, the IL-37 may be the mature form of the human IL-37 peptide, for example after removal of N-terminal amino acids 1 to 46 (of the propeptide sequence) or an equivalent sequence thereof. The IL-37 may be endogenous IL-37 produced from PBMCs. Alternatively, the IL-37 may be from an exogenous source, and was administered to an individual as part of a treatment. In these circumstances, the antigen binding site of the invention may be useful to inhibit or block the activity of the administered exogenous IL-37. Preferably, the antigen binding site binds to or specifically binds to a human IL-37 molecule comprising, consisting essentially of or consisting of an amino acid sequence as shown in SEQ ID NO: 1.

Preferably, the antigen binding site inhibits or reduces the IL-37 activity that is induced by a pro-inflammatory stimulus. The pro-inflammatory stimulus may be any pro-inflammatory stimulus, including for example, bacterial infection (where the stimulus may be the bacterial endotoxin lipopolysaccharide LPS), viral infection, produced by cancer cells, or the result of any other biological, chemical or physical agent or stimulus known to trigger a pro-inflammatory response in an individual.

The IL-37-binding antigen binding site of the present invention may inhibit any IL-37 mediated reduction in cytokine production or secretion from monocytes, macrophages, PBMCs, cancer cells, endothelial cells, epithelial cells, dendritic cells or a cell derived therefrom.

For example, the antigen binding site may increase the production or secretion of IL-1β in PBMCs or a cell type derived therefrom, or cancer cells, compared to when the antigen binding site is not present. Reduction or inhibition of IL-37 activity may be determined by any method as described herein, including by any of the methods described in Examples 4, 5 or 6.

An antigen binding site of the invention may bind to IL-37 and not detectably bind to or bind significantly to IL-18 and/or IL-1β, and/or IL-1Ra, and/or IL-36 and/or IL-36Ra and/or IL-38. The binding of an antigen binding site to IL-18 and/or IL-1β may be determined by any method described herein, particularly immunoblotting as described in Example 3.

The invention provides an antigen binding site for binding to IL-37, the antigen binding site comprising:

FR1-CDR1-FR2-CDR2-FR3-CDR3-FR4-linker-FR1a-CDR1a-FR2a-CDR2a-FR3a-CDR3a-FR4a

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the framework regions or complementarity determining regions are as described herein.

The invention provides an antigen binding site for binding to IL-37, the antigen binding site comprising:

FR1-CDR1-FR2-CDR2-FR3-CDR3-FR4-linker-FR1a-CDR1a-FR2a-CDR2a-FR3a-CDR3a-FR4a

wherein:

FR1, FR2, FR3 and FR4 are each framework regions;

CDR1, CDR2 and CDR3 are each complementarity determining regions;

FR1a, FR2a, FR3a and FR4a are each framework regions;

CDR1a, CDR2a and CDR3a are each complementarity determining regions;

wherein the sequence of any of the complementarity determining regions have an amino acid sequence as described in Table 1 below. Preferably, the framework regions have an amino acid sequence as described in Table 3 below. CDR1, CDR2 and CDR3 may be sequences from the Variable Heavy chain (V_{H}); CDR1a, CDR2a and CDR3a may be sequences from the Variable Light chain (V_{L}). Alternatively, CDR1, CDR2 and CDR3 are sequences from the V_{L}, CDR1a, CDR2a and CDR3a are sequences from V_{H}.

The invention provides an antigen binding site comprising, consisting essentially of or consisting of an amino acids sequence of (in order of N to C terminus or C to N terminus):
SEQ ID NO: 8 and 9;
SEQ ID NO: 18 and 19;
SEQ ID NO: 44 and 45;
SEQ ID NO: 54 and 55;
SEQ ID NO: 64 and 65;
SEQ ID NO: 74 and 75;
SEQ ID NO: 84 and 85;
SEQ ID NO: 94 and 95;
SEQ ID NO: 104 and 105;
SEQ ID NO: 114 and 115;
SEQ ID NO: 124 and 125;
SEQ ID NO: 134 and 135;
SEQ ID NO: 144 and 145;
SEQ ID NO: 154 and 155;
SEQ ID NO: 164 and 165;
SEQ ID NO: 174 and 175;
SEQ ID NO: 184 and 185;
SEQ ID NO: 194 and 195; or
SEQ ID NO: 204 and 205.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:5, a CDR2 comprising a sequence at least about 80%, at least 85%, at least 90%, at least 92%, at least 95%, at least 97%, at least 99% identical to a sequence set in SEQ ID NO:6 and a CDR3 comprising a sequence at least about 80%, at least 85%, at least 90%, at least 92%, at least 95%, at least 97%, at least 99% identical to a sequence set forth in SEQ ID NO: 7;

(ii) a V_{H} comprising a sequence at least about 95% or 96% or 97% or 98% or 99% identical to a sequence set forth in SEQ ID NO: 9;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 2, a CDR2 comprising a sequence at least about 80%, at least 85%, at least 90%, at least 92%, at least 95%, at least 97%, at least 99% identical to a sequence set forth in SEQ ID NO: 3 and a CDR3 comprising a sequence at least about 80%, at least 85%, at least 90%, at least 92%, at least 95%, at least 97%, at least 99% identical to a sequence set forth in SEQ ID NO: 4;

(iv) a V_{L} comprising a sequence at least about 95% identical to a sequence set forth in SEQ ID NO: 8;

(v) a V_{H} comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7;

(vi) a V_{H} comprising a sequence set forth in SEQ ID NO: 9;

(vii) a V_{L} comprising a CDR1 comprising a sequence set SEQ ID NO: 2, a CDR2 comprising a sequence set forth in SEQ ID NO: 3 and a CDR3 comprising a sequence set forth in SEQ ID NO: 4;

(viii) a V_{L} comprising a sequence set forth in SEQ ID NO: 8;

(ix) a V_{H} comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7; and a V_{L} comprising a CDR1 comprising a sequence set SEQ ID NO: 2, a CDR2 comprising a sequence set forth in SEQ ID NO: 3 and a CDR3 comprising a sequence set forth in SEQ ID NO: 4; and

(x) a V_{H} comprising a sequence set forth in SEQ ID NO: 9 and a V_{L} comprising a sequence set forth in SEQ ID NO: 8.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO: 15, a CDR2 comprising a sequence at least about 80%, at least 85%, at least 90%, at least 92%, at least 95%, at least 97%, at least 99% identical to a sequence set in SEQ ID NO: 16 and a CDR3 comprising a sequence at least about 80%, at least 85%, at least 90%, at least 92%, at least 95%, at least 97%, at least 99% identical to a sequence set forth in SEQ ID NO: 17;

(ii) a V_{H} comprising a sequence at least about 95% or 96% or 97% or 98% or 99% identical to a sequence set forth in SEQ ID NO: 19;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 12, a CDR2 comprising a sequence at least about 80%, at least 85%, at least 90%, at least 92%, at least 95%, at least 97%, at least 99% identical to a sequence set forth in SEQ ID NO: 13 and a CDR3 comprising a sequence at least about 80%, at least 85%, at least 90%, at least 92%, at least 95%, at least 97%, at least 99% identical to a sequence set forth in SEQ ID NO: 14;

(iv) a V_{L} comprising a sequence at least about 95% identical to a sequence set forth in SEQ ID NO: 18;

(v) a V_{H} comprising a CDR1 comprising a sequence set forth in SEQ ID NO: 15, a CDR2 comprising a sequence set forth between in SEQ ID NO: 16 and a CDR3 comprising a sequence set forth in SEQ ID NO: 17;

(vi) a V_{H} comprising a sequence set forth in SEQ ID NO: 19;

(vii) a V_{L} comprising a CDR1 comprising a sequence set SEQ ID NO: 12, a CDR2 comprising a sequence set forth in SEQ ID NO: 13 and a CDR3 comprising a sequence set forth in SEQ ID NO: 14;

(viii) a V_{L} comprising a sequence set forth in SEQ ID NO: 18;

(ix) a V_{H} comprising a CDR1 comprising a sequence set forth in SEQ ID NO: 15, a CDR2 comprising a sequence set forth between in SEQ ID NO: 16 and a CDR3 comprising a sequence set forth in SEQ ID NO: 17; and a V_{L} comprising a CDR1 comprising a sequence set SEQ ID NO: 12, a CDR2 comprising a sequence set forth in SEQ ID NO: 13 and a CDR3 comprising a sequence set forth in SEQ ID NO: 14; and

(x) a V_{H} comprising a sequence set forth in SEQ ID NO: 19 and a V_{L} comprising a sequence set forth in SEQ ID NO: 18.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:41, a CDR2 comprising a sequence at least about 80%, at least 85%, at least 90%, at least 92%, at least 95%, at least 97%, at least 99% identical to a sequence set in SEQ ID NO:42 and a CDR3 comprising a sequence at least about 80%, at least 85%, at least 90%, at least 92%, at least 95%, at least 97%, at least 99% identical to a sequence set forth in SEQ ID NO: 43;

(ii) a V_{H} comprising a sequence at least about 95% or 96% or 97% or 98% or 99% identical to a sequence set forth in SEQ ID NO: 45;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 38, a CDR2 comprising a sequence at least about 80%, at least 85%, at least 90%, at least 92%, at least 95%, at least 97%, at least 99% identical to a sequence set forth in SEQ ID NO: 39 and a CDR3 comprising a sequence at least about 80%, at least 85%, at least 90%, at least 92%, at least 95%, at least 97%, at least 99% identical to a sequence set forth in SEQ ID NO: 40;

(iv) a V_{L} comprising a sequence at least about 95% identical to a sequence set forth in SEQ ID NO: 44;

(v) a V_{H} comprising a CDR1 comprising a sequence set forth in SEQ ID NO: 41, a CDR2 comprising a sequence set forth between in SEQ ID NO: 42 and a CDR3 comprising a sequence set forth in SEQ ID NO: 43;

(vi) a V_{H} comprising a sequence set forth in SEQ ID NO: 45;

(vii) a V_{L} comprising a CDR1 comprising a sequence set SEQ ID NO: 38, a CDR2 comprising a sequence set forth in SEQ ID NO: 39 and a CDR3 comprising a sequence set forth in SEQ ID NO: 40;

(viii) a V_{L} comprising a sequence set forth in SEQ ID NO: 44;

(ix) a V_{H} comprising a CDR1 comprising a sequence set forth in SEQ ID NO: 41, a CDR2 comprising a sequence set forth between in SEQ ID NO: 42 and a CDR3 comprising a sequence set forth in SEQ ID NO: 43; and a V_{L} comprising a CDR1 comprising a sequence set SEQ ID NO: 38, a CDR2 comprising a sequence set forth in SEQ ID NO: 39 and a CDR3 comprising a sequence set forth in SEQ ID NO: 40; and

(x) a V_{H} comprising a sequence set forth in SEQ ID NO: 45 and a V_{L} comprising a sequence set forth in SEQ ID NO: 44.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:51, a CDR2 comprising a sequence at least about 80%, at least 85%, at least 90%, at least 92%, at least 95%, at least 97%, at least 99% identical to a sequence set in SEQ ID NO:52 and a CDR3 comprising a sequence at least about 80%, at least 85%, at least 90%, at least 92%, at least 95%, at least 97%, at least 99% identical to a sequence set forth in SEQ ID NO: 53;

(ii) a V_{H} comprising a sequence at least about 95% or 96% or 97% or 98% or 99% identical to a sequence set forth in SEQ ID NO: 55;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 48, a CDR2 comprising a sequence at least about 80%, at least 85%, at least 90%, at least 92%, at least 95%, at least 97%, at least 99% identical to a sequence set forth in SEQ ID NO: 49 and a CDR3 comprising a sequence at least about 80%, at least 85%, at least 90%, at least 92%, at least 95%, at least 97%, at least 99% identical to a sequence set forth in SEQ ID NO: 50;

(iv) a V_{L} comprising a sequence at least about 95% identical to a sequence set forth in SEQ ID NO: 54;

(v) a V_{H} comprising a CDR1 comprising a sequence set forth in SEQ ID NO: 51, a CDR2 comprising a sequence set forth between in SEQ ID NO: 52 and a CDR3 comprising a sequence set forth in SEQ ID NO: 53;

(vi) a V_{H} comprising a sequence set forth in SEQ ID NO: 55;

(vii) a V_{L} comprising a CDR1 comprising a sequence set SEQ ID NO: 48, a CDR2 comprising a sequence set forth in SEQ ID NO: 49 and a CDR3 comprising a sequence set forth in SEQ ID NO: 50;

(viii) a V_{L} comprising a sequence set forth in SEQ ID NO: 54;

(ix) a V_{H} comprising a CDR1 comprising a sequence set forth in SEQ ID NO: 51, a CDR2 comprising a sequence set forth between in SEQ ID NO: 52 and a CDR3 comprising a sequence set forth in SEQ ID NO: 53; and a V_{L} comprising a CDR1 comprising a sequence set SEQ ID NO: 48, a CDR2 comprising a sequence set forth in SEQ ID NO: 49 and a CDR3 comprising a sequence set forth in SEQ ID NO: 50; and

(x) a V_{H} comprising a sequence set forth in SEQ ID NO: 55 and a V_{L} comprising a sequence set forth in SEQ ID NO: 54.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:61, a CDR2 comprising a sequence at least about 80%, at least 85%, at least 90%, at least 92%, at least 95%, at least 97%, at least 99% identical to a sequence set in SEQ ID NO:62 and a CDR3 comprising a sequence at least about 80%, at least 85%, at least 90%, at least 92%, at least 95%, at least 97%, at least 99% identical to a sequence set forth in SEQ ID NO: 63;

(ii) a V_{H} comprising a sequence at least about 95% or 96% or 97% or 98% or 99% identical to a sequence set forth in SEQ ID NO: 65;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 58, a CDR2 comprising a sequence at least about 80%, at least 85%, at least 90%, at least 92%, at least 95%, at least 97%, at least 99% identical to a sequence set forth in SEQ ID NO: 59 and a CDR3 comprising a sequence at least about 80%, at least 85%, at least 90%, at least 92%, at least 95%, at least 97%, at least 99% identical to a sequence set forth in SEQ ID NO: 60;

(iv) a V_{L} comprising a sequence at least about 95% identical to a sequence set forth in SEQ ID NO: 64;

(v) a V_{H} comprising a CDR1 comprising a sequence set forth in SEQ ID NO: 61, a CDR2 comprising a sequence set forth between in SEQ ID NO: 62 and a CDR3 comprising a sequence set forth in SEQ ID NO: 63;

(vi) a V_{H} comprising a sequence set forth in SEQ ID NO: 65;

(vii) a V_{L} comprising a CDR1 comprising a sequence set SEQ ID NO: 58, a CDR2 comprising a sequence set forth in SEQ ID NO: 59 and a CDR3 comprising a sequence set forth in SEQ ID NO: 60;

(viii) a V_{L} comprising a sequence set forth in SEQ ID NO: 64;

(ix) a V_{H} comprising a CDR1 comprising a sequence set forth in SEQ ID NO: 61, a CDR2 comprising a sequence set forth between in SEQ ID NO: 62 and a CDR3 comprising a sequence set forth in SEQ ID NO: 63; and a V_{L} comprising a CDR1 comprising a sequence set SEQ ID NO: 58, a CDR2 comprising a sequence set forth in SEQ ID NO: 59 and a CDR3 comprising a sequence set forth in SEQ ID NO: 60; and

(x) a V_{H} comprising a sequence set forth in SEQ ID NO: 65 and a V_{L} comprising a sequence set forth in SEQ ID NO: 64.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO: 71, a CDR2 comprising a sequence at least about 80%, at least 85%, at least 90%, at least 92%, at least 95%, at least 97%, at least 99% identical to a sequence set in SEQ ID NO:72 and a CDR3 comprising a sequence at least about 80%, at least 85%, at least 90%, at least 92%, at least 95%, at least 97%, at least 99% identical to a sequence set forth in SEQ ID NO: 73;

(ii) a V_{H} comprising a sequence at least about 95% or 96% or 97% or 98% or 99% identical to a sequence set forth in SEQ ID NO: 75;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 68, a CDR2 comprising a sequence at least about 80%, at least 85%, at least 90%, at least 92%, at least 95%, at least 97%, at least 99% identical to a sequence set forth in SEQ ID NO: 69 and a CDR3 comprising a sequence at least about 80%, at least 85%, at least 90%, at least 92%, at least 95%, at least 97%, at least 99% identical to a sequence set forth in SEQ ID NO: 70;

(iv) a V_{L} comprising a sequence at least about 95% identical to a sequence set forth in SEQ ID NO: 74;

(v) a V_{H} comprising a CDR1 comprising a sequence set forth in SEQ ID NO: 71, a CDR2 comprising a sequence set forth between in SEQ ID NO: 72 and a CDR3 comprising a sequence set forth in SEQ ID NO: 73;

(vi) a V_{H} comprising a sequence set forth in SEQ ID NO: 75;

(vii) a V_{L} comprising a CDR1 comprising a sequence set SEQ ID NO: 68, a CDR2 comprising a sequence set forth in SEQ ID NO: 69 and a CDR3 comprising a sequence set forth in SEQ ID NO: 70;

(viii) a V_{L} comprising a sequence set forth in SEQ ID NO: 74;

(ix) a V_{H} comprising a CDR1 comprising a sequence set forth in SEQ ID NO: 71, a CDR2 comprising a sequence set forth between in SEQ ID NO: 72 and a CDR3 comprising a sequence set forth in SEQ ID NO: 73; and a V_{L} comprising a CDR1 comprising a sequence set SEQ ID NO: 68, a CDR2 comprising a sequence set forth in SEQ ID NO: 69 and a CDR3 comprising a sequence set forth in SEQ ID NO: 70; and

(x) a V_{H} comprising a sequence set forth in SEQ ID NO: 75 and a V_{L} comprising a sequence set forth in SEQ ID NO: 74.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:81, a CDR2 comprising a sequence at least about 80%, at least 85%, at least 90%, at least 92%, at least 95%, at least 97%, at least 99% identical to a sequence set in SEQ ID NO: 82 and a CDR3 comprising a sequence at least about 80%, at least 85%, at least 90%, at least 92%, at least 95%, at least 97%, at least 99% identical to a sequence set forth in SEQ ID NO: 83;

(ii) a V_{H} comprising a sequence at least about 95% or 96% or 97% or 98% or 99% identical to a sequence set forth in SEQ ID NO: 85;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 78, a CDR2 comprising a sequence at least about 80%, at least 85%, at least 90%, at least 92%, at least 95%, at least 97%, at least 99% identical to a sequence set forth in SEQ ID NO: 79 and a CDR3 comprising a sequence at least about 80%, at least 85%, at least 90%, at least 92%, at least 95%, at least 97%, at least 99% identical to a sequence set forth in SEQ ID NO: 80;

(iv) a V_{L} comprising a sequence at least about 95% identical to a sequence set forth in SEQ ID NO: 84;

(v) a V_{H} comprising a CDR1 comprising a sequence set forth in SEQ ID NO: 81, a CDR2 comprising a sequence set forth between in SEQ ID NO: 82 and a CDR3 comprising a sequence set forth in SEQ ID NO: 83;

(vi) a V_{H} comprising a sequence set forth in SEQ ID NO: 85;

(vii) a V_{L} comprising a CDR1 comprising a sequence set SEQ ID NO: 78, a CDR2 comprising a sequence set forth in SEQ ID NO: 79 and a CDR3 comprising a sequence set forth in SEQ ID NO: 80;

(viii) a V_{L} comprising a sequence set forth in SEQ ID NO: 84;

(ix) a V_{H} comprising a CDR1 comprising a sequence set forth in SEQ ID NO: 81, a CDR2 comprising a sequence set forth between in SEQ ID NO: 82 and a CDR3 comprising a sequence set forth in SEQ ID NO: 83; and a V_{L} comprising a CDR1 comprising a sequence set SEQ ID NO: 78, a CDR2 comprising a sequence set forth in SEQ ID NO: 79 and a CDR3 comprising a sequence set forth in SEQ ID NO: 80; and

(x) a V_{H} comprising a sequence set forth in SEQ ID NO: 85 and a V_{L} comprising a sequence set forth in SEQ ID NO: 84.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:91, a CDR2 comprising a sequence at least about 80%, at least 85%, at least 90%, at least 92%, at least 95%, at least 97%, at least 99% identical to a sequence set in SEQ ID NO:92 and a CDR3 comprising a sequence at least about 80%, at least 85%, at least 90%, at least 92%, at least 95%, at least 97%, at least 99% identical to a sequence set forth in SEQ ID NO: 93;

(ii) a V_{H} comprising a sequence at least about 95% or 96% or 97% or 98% or 99% identical to a sequence set forth in SEQ ID NO: 95;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 88, a CDR2 comprising a sequence at least about 80%, at least 85%, at least 90%, at least 92%, at least 95%, at least 97%, at least 99% identical to a sequence set forth in SEQ ID NO: 89 and a CDR3 comprising a sequence at least about 80%, at least 85%, at least 90%, at least 92%, at least 95%, at least 97%, at least 99% identical to a sequence set forth in SEQ ID NO: 90;

(iv) a V_{L} comprising a sequence at least about 95% identical to a sequence set forth in SEQ ID NO: 94;

(v) a V_{H} comprising a CDR1 comprising a sequence set forth in SEQ ID NO: 91, a CDR2 comprising a sequence set forth between in SEQ ID NO: 92 and a CDR3 comprising a sequence set forth in SEQ ID NO: 93;

(vi) a V_{H} comprising a sequence set forth in SEQ ID NO: 95;

(vii) a V_{L} comprising a CDR1 comprising a sequence set SEQ ID NO: 88, a CDR2 comprising a sequence set forth in SEQ ID NO: 89 and a CDR3 comprising a sequence set forth in SEQ ID NO: 90;

(viii) a V_{L} comprising a sequence set forth in SEQ ID NO: 94;

(ix) a V_{H} comprising a CDR1 comprising a sequence set forth in SEQ ID NO: 91, a CDR2 comprising a sequence set forth between in SEQ ID NO: 92 and a CDR3 comprising a sequence set forth in SEQ ID NO: 93; and a V_{L} comprising a CDR1 comprising a sequence set SEQ ID NO: 88, a CDR2 comprising a sequence set forth in SEQ ID NO: 89 and a CDR3 comprising a sequence set forth in SEQ ID NO: 90; and

(x) a V_{H} comprising a sequence set forth in SEQ ID NO: 95 and a V_{L} comprising a sequence set forth in SEQ ID NO: 94.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:101, a CDR2 comprising a sequence at least about 80%, at least 85%, at least 90%, at least 92%, at least 95%, at least 97%, at least 99% identical to a sequence set in SEQ ID NO:102 and a CDR3 comprising a sequence at least about 80%, at least 85%, at least 90%, at least 92%, at least 95%, at least 97%, at least 99% identical to a sequence set forth in SEQ ID NO: 103;

(ii) a V_{H} comprising a sequence at least about 95% or 96% or 97% or 98% or 99% identical to a sequence set forth in SEQ ID NO: 105;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 98, a CDR2 comprising a sequence at least about 80%, at least 85%, at least 90%, at least 92%, at least 95%, at least 97%, at least 99% identical to a sequence set forth in SEQ ID NO: 99 and a CDR3 comprising a sequence at least about 80%, at least 85%, at least 90%, at least 92%, at least 95%, at least 97%, at least 99% identical to a sequence set forth in SEQ ID NO: 100;

(iv) a V_{L} comprising a sequence at least about 95% identical to a sequence set forth in SEQ ID NO: 104;

(v) a V_{H} comprising a CDR1 comprising a sequence set forth in SEQ ID NO: 101, a CDR2 comprising a sequence set forth between in SEQ ID NO: 102 and a CDR3 comprising a sequence set forth in SEQ ID NO: 103;

(vi) a V_{H} comprising a sequence set forth in SEQ ID NO: 105;

(vii) a V_{L} comprising a CDR1 comprising a sequence set SEQ ID NO: 98, a CDR2 comprising a sequence set forth in SEQ ID NO: 99 and a CDR3 comprising a sequence set forth in SEQ ID NO: 100;

(viii) a V_{L} comprising a sequence set forth in SEQ ID NO: 104;

(ix) a V_{H} comprising a CDR1 comprising a sequence set forth in SEQ ID NO: 101, a CDR2 comprising a sequence set forth between in SEQ ID NO: 102 and a CDR3 comprising a sequence set forth in SEQ ID NO: 103; and a V_{L} comprising a CDR1 comprising a sequence set SEQ ID NO: 98, a CDR2 comprising a sequence set forth in SEQ ID NO: 99 and a CDR3 comprising a sequence set forth in SEQ ID NO: 100; and

(x) a V_{H} comprising a sequence set forth in SEQ ID NO: 105 and a V_{L} comprising a sequence set forth in SEQ ID NO: 104.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:111, a CDR2 comprising a sequence at least about 80%, at least 85%, at least 90%, at least 92%, at least 95%, at least 97%, at least 99% identical to a sequence set in SEQ ID NO:112 and a CDR3 comprising a sequence at least about 80%, at least 85%, at least 90%, at least 92%, at least 95%, at least 97%, at least 99% identical to a sequence set forth in SEQ ID NO: 113;

(ii) a V_{H} comprising a sequence at least about 95% or 96% or 97% or 98% or 99% identical to a sequence set forth in SEQ ID NO: 115;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 108, a CDR2 comprising a sequence at least about 80%, at least 85%, at least 90%, at least 92%, at least 95%, at least 97%, at least 99% identical to a sequence set forth in SEQ ID NO: 109 and a CDR3 comprising a sequence at least about 80%, at least 85%, at least 90%, at least 92%, at least 95%, at least 97%, at least 99% identical to a sequence set forth in SEQ ID NO: 110;

(iv) a V_{L} comprising a sequence at least about 95% identical to a sequence set forth in SEQ ID NO: 114;

(v) a V_{H} comprising a CDR1 comprising a sequence set forth in SEQ ID NO: 111, a CDR2 comprising a sequence set forth between in SEQ ID NO: 112 and a CDR3 comprising a sequence set forth in SEQ ID NO: 113;

(vi) a V_{H} comprising a sequence set forth in SEQ ID NO: 115;

(vii) a V_{L} comprising a CDR1 comprising a sequence set SEQ ID NO: 108, a CDR2 comprising a sequence set forth in SEQ ID NO: 109 and a CDR3 comprising a sequence set forth in SEQ ID NO: 110;

(viii) a V_{L} comprising a sequence set forth in SEQ ID NO: 114;

(ix) a V_{H} comprising a CDR1 comprising a sequence set forth in SEQ ID NO: 111, a CDR2 comprising a sequence set forth between in SEQ ID NO: 112 and a CDR3 comprising a sequence set forth in SEQ ID NO: 113; and a V_{L} comprising a CDR1 comprising a sequence set SEQ ID NO: 108, a CDR2 comprising a sequence set forth in SEQ ID NO: 109 and a CDR3 comprising a sequence set forth in SEQ ID NO: 110; and

(x) a V_{H} comprising a sequence set forth in SEQ ID NO: 115 and a V_{L} comprising a sequence set forth in SEQ ID NO: 114.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:121, a CDR2 comprising a sequence at least about 80%, at least 85%, at least 90%, at least 92%, at least 95%, at least 97%, at least 99% identical to a sequence set in SEQ ID NO:122 and a CDR3 comprising a sequence at least about 80%, at least 85%, at least 90%, at least 92%, at least 95%, at least 97%, at least 99% identical to a sequence set forth in SEQ ID NO: 123;

(ii) a V_{H} comprising a sequence at least about 95% or 96% or 97% or 98% or 99% identical to a sequence set forth in SEQ ID NO: 125;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 118, a CDR2 comprising a sequence at least about 80%, at least 85%, at least 90%, at least 92%, at least 95%, at least 97%, at least 99% identical to a sequence set forth in SEQ ID NO: 119 and a CDR3 comprising a sequence at least about 80%, at least 85%, at least 90%, at least 92%, at least 95%, at least 97%, at least 99% identical to a sequence set forth in SEQ ID NO: 120;

(iv) a V_{L} comprising a sequence at least about 95% identical to a sequence set forth in SEQ ID NO: 124;

(v) a V_{H} comprising a CDR1 comprising a sequence set forth in SEQ ID NO: 121, a CDR2 comprising a sequence set forth between in SEQ ID NO: 122 and a CDR3 comprising a sequence set forth in SEQ ID NO: 123;

(vi) a V_{H} comprising a sequence set forth in SEQ ID NO: 125;

(vii) a V_{L} comprising a CDR1 comprising a sequence set SEQ ID NO: 118, a CDR2 comprising a sequence set forth in SEQ ID NO: 119 and a CDR3 comprising a sequence set forth in SEQ ID NO: 120;

(viii) a V_{L} comprising a sequence set forth in SEQ ID NO: 124;

(ix) a V_{H} comprising a CDR1 comprising a sequence set forth in SEQ ID NO: 121, a CDR2 comprising a sequence set forth between in SEQ ID NO: 122 and a CDR3 comprising a sequence set forth in SEQ ID NO: 123; and a V_{L} comprising a CDR1 comprising a sequence set SEQ ID NO: 118, a CDR2 comprising a sequence set forth in SEQ ID NO: 119 and a CDR3 comprising a sequence set forth in SEQ ID NO: 120; and

(x) a V_{H} comprising a sequence set forth in SEQ ID NO: 125 and a V_{L} comprising a sequence set forth in SEQ ID NO: 124.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:131, a CDR2 comprising a sequence at least about 80%, at least 85%, at least 90%, at least 92%, at least 95%, at least 97%, at least 99% identical to a sequence set in SEQ ID NO:132 and a CDR3 comprising a sequence at least about 80%, at least 85%, at least 90%, at least 92%, at least 95%, at least 97%, at least 99% identical to a sequence set forth in SEQ ID NO: 133;

(ii) a V_{H} comprising a sequence at least about 95% or 96% or 97% or 98% or 99% identical to a sequence set forth in SEQ ID NO: 135;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 128, a CDR2 comprising a sequence at least about 80%, at least 85%, at least 90%, at least 92%, at least 95%, at least 97%, at least 99% identical to a sequence set forth in SEQ ID NO: 129 and a CDR3 comprising a sequence at least about 80%, at least 85%, at least 90%, at least 92%, at least 95%, at least 97%, at least 99% identical to a sequence set forth in SEQ ID NO: 130;

(iv) a V_{L} comprising a sequence at least about 95% identical to a sequence set forth in SEQ ID NO: 134;

(v) a V_{H} comprising a CDR1 comprising a sequence set forth in SEQ ID NO: 131, a CDR2 comprising a sequence set forth between in SEQ ID NO: 132 and a CDR3 comprising a sequence set forth in SEQ ID NO: 133;

(vi) a V_{H} comprising a sequence set forth in SEQ ID NO: 135;

(vii) a V_{L} comprising a CDR1 comprising a sequence set SEQ ID NO: 128, a CDR2 comprising a sequence set forth in SEQ ID NO: 129 and a CDR3 comprising a sequence set forth in SEQ ID NO: 130;

(viii) a V_{L} comprising a sequence set forth in SEQ ID NO: 134;

(ix) a V_{H} comprising a CDR1 comprising a sequence set forth in SEQ ID NO: 131, a CDR2 comprising a sequence set forth between in SEQ ID NO: 132 and a CDR3 comprising a sequence set forth in SEQ ID NO: 133; and a V_{L} comprising a CDR1 comprising a sequence set SEQ ID NO: 128, a CDR2 comprising a sequence set forth in SEQ ID NO: 129 and a CDR3 comprising a sequence set forth in SEQ ID NO: 130; and

(x) a V_{H} comprising a sequence set forth in SEQ ID NO: 135 and a V_{L} comprising a sequence set forth in SEQ ID NO: 134.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:141, a CDR2 comprising a sequence at least about 80%, at least 85%, at least 90%, at least 92%, at least 95%, at least 97%, at least 99% identical to a sequence set in SEQ ID NO:142 and a CDR3 comprising a sequence at least about 80%, at least 85%, at least 90%, at least 92%, at least 95%, at least 97%, at least 99% identical to a sequence set forth in SEQ ID NO: 143;

(ii) a V_{H} comprising a sequence at least about 95% or 96% or 97% or 98% or 99% identical to a sequence set forth in SEQ ID NO: 145;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 138, a CDR2 comprising a sequence at least about 80%, at least 85%, at least 90%, at least 92%, at least 95%, at least 97%, at least 99% identical to a sequence set forth in SEQ ID NO: 139 and a CDR3 comprising a sequence at least about 80%, at least 85%, at least 90%, at least 92%, at least 95%, at least 97%, at least 99% identical to a sequence set forth in SEQ ID NO: 140;

(iv) a V_{L} comprising a sequence at least about 95% identical to a sequence set forth in SEQ ID NO: 144;

(v) a V_{H} comprising a CDR1 comprising a sequence set forth in SEQ ID NO: 141, a CDR2 comprising a sequence set forth between in SEQ ID NO: 142 and a CDR3 comprising a sequence set forth in SEQ ID NO: 143;

(vi) a V_{H} comprising a sequence set forth in SEQ ID NO: 145;

(vii) a V_{L} comprising a CDR1 comprising a sequence set SEQ ID NO: 138, a CDR2 comprising a sequence set forth in SEQ ID NO: 139 and a CDR3 comprising a sequence set forth in SEQ ID NO: 140;

(viii) a V_{L} comprising a sequence set forth in SEQ ID NO: 144;

(ix) a V_{H} comprising a CDR1 comprising a sequence set forth in SEQ ID NO: 141, a CDR2 comprising a sequence set forth between in SEQ ID NO: 142 and a CDR3 comprising a sequence set forth in SEQ ID NO: 143; and a V_{L} comprising a CDR1 comprising a sequence set SEQ ID NO: 138, a CDR2 comprising a sequence set forth in SEQ ID NO: 139 and a CDR3 comprising a sequence set forth in SEQ ID NO: 140; and

(x) a V_{H} comprising a sequence set forth in SEQ ID NO: 145 and a V_{L} comprising a sequence set forth in SEQ ID NO: 144.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:151, a CDR2 comprising a sequence at least about 80%, at least 85%, at least 90%, at least 92%, at least 95%, at least 97%, at least 99% identical to a sequence set in SEQ ID NO:152 and a CDR3 comprising a sequence at least about 80%, at least 85%, at least 90%, at least 92%, at least 95%, at least 97%, at least 99% identical to a sequence set forth in SEQ ID NO: 153;

(ii) a V_{H} comprising a sequence at least about 95% or 96% or 97% or 98% or 99% identical to a sequence set forth in SEQ ID NO: 155;

(iii) a V_{L} comprising a CDR1 comprising a sequence at least about 80%, at least 85%, at least 90%, at least 92%, at least 95%, at least 97%, at least 99% identical to a sequence set forth in SEQ ID NO:148, a CDR2 comprising a sequence at least about 80%, at least 85%, at least 90%, at least 92%, at least 95%, at least 97%, at least 99% identical to a sequence set forth in SEQ ID NO: 149 and a CDR3 comprising a sequence at least about 80%, at least 85%, at least 90%, at least 92%, at least 95%, at least 97%, at least 99% identical to a sequence set forth in SEQ ID NO: 150;

(iv) a V_{L} comprising a sequence at least about 95% identical to a sequence set forth in SEQ ID NO: 154;

(v) a V_{H} comprising a CDR1 comprising a sequence set forth in SEQ ID NO: 151, a CDR2 comprising a sequence set forth between in SEQ ID NO:152 and a CDR3 comprising a sequence set forth in SEQ ID NO: 153;

(vi) a V_{H} comprising a sequence set forth in SEQ ID NO: 155;

(vii) a V_{L} comprising a CDR1 comprising a sequence set SEQ ID NO:1482, a CDR2 comprising a sequence set forth in SEQ ID NO: 149 and a CDR3 comprising a sequence set forth in SEQ ID NO: 150;

(viii) a V_{L} comprising a sequence set forth in SEQ ID NO: 154;

(ix) a V_{H} comprising a CDR1 comprising a sequence set forth in SEQ ID NO: 151, a CDR2 comprising a sequence set forth between in SEQ ID NO: 152 and a CDR3 comprising a sequence set forth in SEQ ID NO: 153; and a V_{L} comprising a CDR1 comprising a sequence set SEQ ID NO: 148, a CDR2 comprising a sequence set forth in SEQ ID NO: 149 and a CDR3 comprising a sequence set forth in SEQ ID NO: 150; and

(x) a V_{H} comprising a sequence set forth in SEQ ID NO: 155 and a V_{L} comprising a sequence set forth in SEQ ID NO: 154.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:161, a CDR2 comprising a sequence at least about 80%, at least 85%, at least 90%, at least 92%, at least 95%, at least 97%, at least 99% identical to a sequence set in SEQ ID NO:162 and a CDR3 comprising a sequence at least about 80%, at least 85%, at least 90%, at least 92%, at least 95%, at least 97%, at least 99% identical to a sequence set forth in SEQ ID NO: 163;

(ii) a V_{H} comprising a sequence at least about 95% or 96% or 97% or 98% or 99% identical to a sequence set forth in SEQ ID NO: 165;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 158, a CDR2 comprising a sequence at least about 80%, at least 85%, at least 90%, at least 92%, at least 95%, at least 97%, at least 99% identical to a sequence set forth in SEQ ID NO: 159 and a CDR3 comprising a sequence at least about 80%, at least 85%, at least 90%, at least 92%, at least 95%, at least 97%, at least 99% identical to a sequence set forth in SEQ ID NO: 160;

(iv) a V_{L} comprising a sequence at least about 95% identical to a sequence set forth in SEQ ID NO: 164;

(v) a V_{H} comprising a CDR1 comprising a sequence set forth in SEQ ID NO: 161, a CDR2 comprising a sequence set forth between in SEQ ID NO: 162 and a CDR3 comprising a sequence set forth in SEQ ID NO: 163;

(vi) a V_{H} comprising a sequence set forth in SEQ ID NO: 165;

(vii) a V_{L} comprising a CDR1 comprising a sequence set SEQ ID NO: 158, a CDR2 comprising a sequence set forth in SEQ ID NO: 159 and a CDR3 comprising a sequence set forth in SEQ ID NO: 160;

(viii) a V_{L} comprising a sequence set forth in SEQ ID NO: 164;

(ix) a V_{H} comprising a CDR1 comprising a sequence set forth in SEQ ID NO: 161, a CDR2 comprising a sequence set forth between in SEQ ID NO: 162 and a CDR3 comprising a sequence set forth in SEQ ID NO: 163; and a V_{L} comprising a CDR1 comprising a sequence set SEQ ID NO:158, a CDR2 comprising a sequence set forth in SEQ ID NO: 159 and a CDR3 comprising a sequence set forth in SEQ ID NO: 160; and

(x) a V_{H} comprising a sequence set forth in SEQ ID NO: 165 and a V_{L} comprising a sequence set forth in SEQ ID NO: 164.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:171, a CDR2 comprising a sequence at least about 80%, at least 85%, at least 90%, at least 92%, at least 95%, at least 97%, at least 99% identical to a sequence set in SEQ ID NO:172 and a CDR3 comprising a sequence at least about 80%, at least 85%, at least 90%, at least 92%, at least 95%, at least 97%, at least 99% identical to a sequence set forth in SEQ ID NO: 173;

(ii) a V_{H} comprising a sequence at least about 95% or 96% or 97% or 98% or 99% identical to a sequence set forth in SEQ ID NO: 175;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 168, a CDR2 comprising a sequence at least about 80%, at least 85%, at least 90%, at least 92%, at least 95%, at least 97%, at least 99% identical to a sequence set forth in SEQ ID NO: 169 and a CDR3 comprising a sequence at least about 80%, at least 85%, at least 90%, at least 92%, at least 95%, at least 97%, at least 99% identical to a sequence set forth in SEQ ID NO: 170;

(iv) a V_{L} comprising a sequence at least about 95% identical to a sequence set forth in SEQ ID NO: 174;

(v) a V_{H} comprising a CDR1 comprising a sequence set forth in SEQ ID NO: 171, a CDR2 comprising a sequence set forth between in SEQ ID NO: 172 and a CDR3 comprising a sequence set forth in SEQ ID NO: 173;

(vi) a V_{H} comprising a sequence set forth in SEQ ID NO: 175;

(vii) a V_{L} comprising a CDR1 comprising a sequence set SEQ ID NO: 168, a CDR2 comprising a sequence set forth in SEQ ID NO: 169 and a CDR3 comprising a sequence set forth in SEQ ID NO: 170;

(viii) a V_{L} comprising a sequence set forth in SEQ ID NO: 174;

(ix) a V_{H} comprising a CDR1 comprising a sequence set forth in SEQ ID NO: 171, a CDR2 comprising a sequence set forth between in SEQ ID NO: 172 and a CDR3 comprising a sequence set forth in SEQ ID NO: 173; and a V_{L} comprising a CDR1 comprising a sequence set SEQ ID NO: 168, a CDR2 comprising a sequence set forth in SEQ ID NO: 169 and a CDR3 comprising a sequence set forth in SEQ ID NO: 170; and

(x) a V_{H} comprising a sequence set forth in SEQ ID NO: 175 and a V_{L} comprising a sequence set forth in SEQ ID NO: 174.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:181, a CDR2 comprising a sequence at least about 80%, at least 85%, at least 90%, at least 92%, at least 95%, at least 97%, at least 99% identical to a sequence set in SEQ ID NO:182 and a CDR3 comprising a sequence at least about 80%, at least 85%, at least 90%, at least 92%, at least 95%, at least 97%, at least 99% identical to a sequence set forth in SEQ ID NO: 183;

(ii) a V_{H} comprising a sequence at least about 95% or 96% or 97% or 98% or 99% identical to a sequence set forth in SEQ ID NO: 185;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 178, a CDR2 comprising a sequence at least about 80%, at least 85%, at least 90%, at least 92%, at least 95%, at least 97%, at least 99% identical to a sequence set forth in SEQ ID NO: 179 and a CDR3 comprising a sequence at least about 80%, at least 85%, at least 90%, at least 92%, at least 95%, at least 97%, at least 99% identical to a sequence set forth in SEQ ID NO: 180;

(iv) a V_{L} comprising a sequence at least about 95% identical to a sequence set forth in SEQ ID NO: 184;

(v) a V_{H} comprising a CDR1 comprising a sequence set forth in SEQ ID NO: 181, a CDR2 comprising a sequence set forth between in SEQ ID NO: 182 and a CDR3 comprising a sequence set forth in SEQ ID NO: 183;

(vi) a V_{H} comprising a sequence set forth in SEQ ID NO: 185;

(vii) a V_{L} comprising a CDR1 comprising a sequence set SEQ ID NO:178, a CDR2 comprising a sequence set forth in SEQ ID NO: 179 and a CDR3 comprising a sequence set forth in SEQ ID NO: 180

(viii) a V_{L} comprising a sequence set forth in SEQ ID NO: 184;

(ix) a V_{H} comprising a CDR1 comprising a sequence set forth in SEQ ID NO: 181, a CDR2 comprising a sequence set forth between in SEQ ID NO: 182 and a CDR3 comprising a sequence set forth in SEQ ID NO: 183; and a V_{L} comprising a CDR1 comprising a sequence set SEQ ID NO: 178, a CDR2 comprising a sequence set forth in SEQ ID NO: 179 and a CDR3 comprising a sequence set forth in SEQ ID NO: 180; and

(x) a V_{H} comprising a sequence set forth in SEQ ID NO: 185 and a V_{L} comprising a sequence set forth in SEQ ID NO: 184.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:191, a CDR2 comprising a sequence at least about 80%, at least 85%, at least 90%, at least 92%, at least 95%, at least 97%, at least 99% identical to a sequence set in SEQ ID NO:192 and a CDR3 comprising a sequence at least about 80%, at least 85%, at least 90%, at least 92%, at least 95%, at least 97%, at least 99% identical to a sequence set forth in SEQ ID NO: 193;

(ii) a V_{H} comprising a sequence at least about 95% or 96% or 97% or 98% or 99% identical to a sequence set forth in SEQ ID NO: 195;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 188, a CDR2 comprising a sequence at least about 80%, at least 85%, at least 90%, at least 92%, at least 95%, at least 97%, at least 99% identical to a sequence set forth in SEQ ID NO: 189 and a CDR3 comprising a sequence at least about 80%, at least 85%, at least 90%, at least 92%, at least 95%, at least 97%, at least 99% identical to a sequence set forth in SEQ ID NO: 190;

(iv) a V_{L} comprising a sequence at least about 95% identical to a sequence set forth in SEQ ID NO: 194;

(v) a V_{H} comprising a CDR1 comprising a sequence set forth in SEQ ID NO: 191, a CDR2 comprising a sequence set forth between in SEQ ID NO: 192 and a CDR3 comprising a sequence set forth in SEQ ID NO: 193;

(vi) a V_{H} comprising a sequence set forth in SEQ ID NO: 195;

(vii) a V_{L} comprising a CDR1 comprising a sequence set SEQ ID NO: 188, a CDR2 comprising a sequence set forth in SEQ ID NO: 189 and a CDR3 comprising a sequence set forth in SEQ ID NO: 190;

(viii) a V_{L} comprising a sequence set forth in SEQ ID NO: 194;

(ix) a V_{H} comprising a CDR1 comprising a sequence set forth in SEQ ID NO: 191, a CDR2 comprising a sequence set forth between in SEQ ID NO: 192 and a CDR3 comprising a sequence set forth in SEQ ID NO: 193; and a V_{L} comprising a CDR1 comprising a sequence set SEQ ID NO: 188, a CDR2 comprising a sequence set forth in SEQ ID NO: 189 and a CDR3 comprising a sequence set forth in SEQ ID NO: 190; and

(x) a V_{H} comprising a sequence set forth in SEQ ID NO: 195 and a V_{L} comprising a sequence set forth in SEQ ID NO: 194.

The present invention also provides an antigen binding site comprising an antigen binding domain of an antibody, wherein the antigen binding domain binds to or specifically binds to IL-37, wherein the antigen binding domain comprises at least one of:

(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:201, a CDR2 comprising a sequence at least about 80%, at least 85%, at least 90%, at least 92%, at least 95%, at least 97%, at least 99% identical to a sequence set in SEQ ID NO:202 and a CDR3 comprising a sequence at least about 80%, at least 85%, at least 90%, at least 92%, at least 95%, at least 97%, at least 99% identical to a sequence set forth in SEQ ID NO: 203;

(ii) a V_{H} comprising a sequence at least about 95% or 96% or 97% or 98% or 99% identical to a sequence set forth in SEQ ID NO: 205;

(iii) a V_{L} comprising a CDR1 comprising a sequence at least about 80%, at least 85%, at least 90%, at least 92%, at least 95%, at least 97%, at least 99% identical to a sequence set forth in SEQ ID NO: 198, a CDR2 comprising a sequence at least about 80%, at least 85%, at least 90%, at least 92%, at least 95%, at least 97%, at least 99% identical to a sequence set forth in SEQ ID NO: 199 and a CDR3 comprising a sequence at least about 80%, at least 85%, at least 90%, at least 92%, at least 95%, at least 97%, at least 99% identical to a sequence set forth in SEQ ID NO: 200;

(iv) a V_{L} comprising a sequence at least about 95% identical to a sequence set forth in SEQ ID NO: 204;

(v) a V_{H} comprising a CDR1 comprising a sequence set forth in SEQ ID NO: 201, a CDR2 comprising a sequence set forth between in SEQ ID NO: 202 and a CDR3 comprising a sequence set forth in SEQ ID NO: 203;

(vi) a V_{H} comprising a sequence set forth in SEQ ID NO: 205;

(vii) a V_{L} comprising a CDR1 comprising a sequence set SEQ ID NO: 198, a CDR2 comprising a sequence set forth in SEQ ID NO: 199 and a CDR3 comprising a sequence set forth in SEQ ID NO: 200;

(viii) a V_{L} comprising a sequence set forth in SEQ ID NO: 204;

(ix) a V_{H} comprising a CDR1 comprising a sequence set forth in SEQ ID NO: 201, a CDR2 comprising a sequence set forth between in SEQ ID NO: 202 and a CDR3 comprising a sequence set forth in SEQ ID NO: 203; and a V_{L} comprising a CDR1 comprising a sequence set SEQ ID NO: 198, a CDR2 comprising a sequence set forth in SEQ ID NO: 199 and a CDR3 comprising a sequence set forth in SEQ ID NO: 200; and

(x) a V_{H} comprising a sequence set forth in SEQ ID NO: 205 and a V_{L} comprising a sequence set forth in SEQ ID NO: 204.

As described herein, the antigen binding site may be in the form of:

(i) a single chain Fv fragment (scFv);

(ii) a dimeric scFv (di-scFv);

(iii) one of (i) or (ii) linked to a constant region of an antibody, Fc or a heavy chain constant domain (CH) 2 and/or CH3; or

(iv) one of (i) or (ii) linked to a protein that binds to an immune effector cell.

Further, as described herein, the antigen binding site may be in the form of:

(i) a diabody;

(ii) a triabody;

(iii) a tetrabody;

(iv) a Fab;

(v) a F(ab′)2;

(vi) a Fv;

(vii) one of (i) to (vi) linked to a constant region of an antibody, Fc or a heavy chain constant domain (CH) 2 and/or CH3;

(viii) one of (i) to (vi) linked to a protein that binds to an immune effector cell.

The foregoing antigen binding sites can also be referred to as antigen binding domains of antibodies.

An antigen binding site as described herein may be an antibody or antigen binding fragment thereof. Typically, the antigen binding site is an antibody, for example, a monoclonal antibody.

As used herein the antigen binding site may be a variable domain.

In any aspect or embodiment, the antibody is a naked antibody. Specifically, the antibody is in a non-conjugated form and is not adapted to form a conjugate.

In one example, the complementarity determining region sequences (CDRs) of an antigen binding site of the invention are defined according to the Kabat numbering system.

In another example, the CDRs are defined according to the IMGT numbering system.

Reference herein to a protein or antibody that “binds to” IL-37 provides literal support for a protein or antibody that “binds specifically to” or “specifically binds to” IL-37.

The present invention also provides antigen binding domains or antigen binding fragments of the foregoing antibodies.

The invention also provides a fusion protein comprising an antigen binding site, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

The invention also provides a conjugate in the form of an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent.

The invention also provides an antibody for binding to an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

The invention also provides a nucleic acid encoding an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein. As such, the present invention also provides a nucleic acid comprising, consisting essentially of, or consisting of any one of the nucleotide sequences provided in Table 2.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding sites, the expression construct may comprise a promoter linked to a nucleic acid encoding that polypeptide chain.

In examples directed to multiple polypeptide chains that form an antigen binding site, an expression construct comprises a nucleic acid encoding a polypeptide comprising, e.g., a V_{H} operably linked to a promoter and a nucleic acid encoding a polypeptide comprising, e.g., a V_{L} operably linked to a promoter.

In another example, the expression construct is a bicistronic expression construct, e.g., comprising the following operably linked components in 5′ to 3′ order:

(i) a promoter

(ii) a nucleic acid encoding a first polypeptide;

(iii) an internal ribosome entry site; and

(iv) a nucleic acid encoding a second polypeptide,

wherein the first polypeptide comprises a V_{H} and the second polypeptide comprises a V_{L}, or vice versa.

The present invention also contemplates separate expression constructs one of which encodes a first polypeptide comprising a V_{H} and another of which encodes a second polypeptide comprising a V_{L}. For example, the present invention also provides a composition comprising:

(i) a first expression construct comprising a nucleic acid encoding a polypeptide comprising a V_{H} operably linked to a promoter; and

(ii) a second expression construct comprising a nucleic acid encoding a polypeptide comprising a V_{L} operably linked to a promoter.

The invention provides a cell comprising a vector or nucleic acid described herein. Preferably, the cell is isolated, substantially purified or recombinant. In one example, the cell comprises the expression construct of the invention or:

(i) a first expression construct comprising a nucleic acid encoding a polypeptide comprising a V_{H} operably linked to a promoter; and

(ii) a second expression construct comprising a nucleic acid encoding a polypeptide comprising a V_{L} operably linked to a promoter,

wherein the first and second polypeptides associate to form an antigen binding site of the present invention.

Examples of cells of the present invention include bacterial cells, yeast cells, insect cells or mammalian cells.

The invention also provides a pharmaceutical composition comprising an antigen binding site, or comprising a CDR and/or FR sequence as described herein, or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding site, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label.

The invention also provides a kit or article of manufacture comprising an antigen binding site, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

An antigen binding site, a protein or antibody as described herein may comprise a human constant region, e.g., an IgG constant region, such as an IgG1, IgG2, IgG3 or IgG4 constant region or mixtures thereof. In the case of an antibody or protein comprising a V_{H} and a V_{L}, the V_{H} can be linked to a heavy chain constant region and the V_{L} can be linked to a light chain constant region.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington D.C. United States Department of Health and Human Services, 1987 and/or 1991)).

In one example a protein or antibody as described herein or a composition of a protein or antibody as described herein, comprises a heavy chain constant region, comprising a stabilized heavy chain constant region, comprising a mixture of sequences fully or partially with or without the C-terminal lysine residue.

In one example, an antibody of the invention comprises a V_{H} disclosed herein linked or fused to an IgG4 constant region or stabilized IgG4 constant region (e.g., as discussed above) and the V_{L} is linked to or fused to a kappa light chain constant region.

The functional characteristics of an antigen binding site of the invention will be taken to apply mutatis mutandis to an antibody of the invention.

An antigen binding site as described herein may be purified, substantially purified, isolated and/or recombinant.

An antigen binding site of the invention may be part of a supernatant taken from media in which a hybridoma expressing an antigen binding site of the invention has been grown.

The present invention also provides a method for treating or preventing a condition associated with elevated expression, production, activation and/or secretion of endogenous IL-37 in a subject, the method comprising administering an antigen binding site of the invention to the subject, thereby treating or preventing a condition associated with elevated expression, production, activation and/or secretion of endogenous IL-37.

The present invention also provides a method for treating or preventing cancer in a subject, the method comprising administering an antigen binding site of the invention to the subject, thereby treating or preventing cancer. In this regard, an antigen binding site can be used to prevent a relapse of a condition, and this is considered preventing the condition.

Exemplary cancers include hematologic cancers, cancers of epithelial origin, liver cancer, pancreatic cancer, gastric cancer, osteosarcoma, endometrial cancer and ovarian cancer, or any other cancer or type of tumour described herein.

The present invention also provides a method for treating or preventing a condition associated with immune paralysis in a subject, the method comprising administering an antigen binding site of the invention to the subject, thereby treating or preventing a condition associated with immune paralysis. Exemplary conditions associated with immune paralysis include sepsis, and acute and chronic liver failure.

The invention also provides a cell comprising a vector or nucleic acid molecule described herein.

The invention also provides an animal or tissue derived therefrom comprising a cell described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Post fusion supernatant human IL-37 ELISA results. After assessing the binding of antibodies to the target antigen by microarray, the positive clones were further tested by ELISA. The results shown are the absorbance readings (Raw absorbance readings) for binding by ELISA to recombinant human IL-37 and GST as a negative control. The Positive:Negative ratios described were calculated and the positive clones were identified as C3, D3 and A3.

FIG. 2. Immunoblot of hybridoma supernatant for recombinant (rec) IL-18, rec IL-1 and rec IL-37 for detection of specificity and cross reactivity. Blot was probed with anti-IL-37 hybridoma supernatant from clone D3. No cross reactivity with the proteins tested was been observed—Lane 1: rec IL-18, lane 2: IL-10, lane 3: markers (25 KDa marker highlighted), and lane 4: rec human IL-37.

FIG. 3. Blocking of endogenous IL-37 induced by LPS with IL-1β ELISA readout. PBMC freshly isolated from healthy volunteers were incubated with the indicated concentration of hybridoma clone D3 supernatant. Thirty minutes thereafter, cultures were stimulated with LPS (500 pg/ml) or vehicle. Supernatants were collected 0, 1, 3 and 6 h after addition of LPS and IL-1β was measured by ELISA. Graph shows supernatant IL-1β in pg/ml±SEM (n=3 donors). **, P<0.01; ***, P<0.001 compared to LPS alone. ###, P<0.001 compared to LPS+37bl1 one h after t₀.

FIG. 4. Characterization of IL-37-blocking antibody. Freshly isolated PBMC were first treated with the indicated dilutions of the IL-37-blocking antibody (clone D3) or control, followed by addition of the indicated concentrations of D73K⁴⁶⁻²¹⁸ or vehicle 30 min later, and 50 pg/ml LPS another 30 min later. IL-1β protein abundance was determined by ELISA in the culture supernatants 20 h after addition of LPS, and percent changes afforded by treatment with the IL-37b variants in the presence or absence of the IL-37-blocking antibody were calculated and graphed±SEM; n=2 biological replicates from one exemplary donor.

FIG. 5. Blocking of natural or monomeric IL-37. Constructs expressing Natural¹⁻²¹⁸, D73K¹⁻²¹⁸ or Y85A¹⁻²¹⁸ were transfected into THP-1 cells. Following differentiation with PMA (50 ng/ml) for 24 h and a medium change, cultures were pre-treated with an IL-37-blocking antibody (1:50 dilution; clone D3) or control, then stimulated with 250 ng/ml LPS or vehicle. The fold-changes in IL-1β protein abundance between pairs of cultures treated with the IL-37-blocking antibody and vehicle-treated cultures (transfected with identical constructs, i.e. pairs of Control, Natural, D73K, or Y85A, and stimulated with LPS) were calculated and are graphed as mean±SEM; n=4 biological repeats for each LPS-stimulated condition; statistical significance was assessed using ANOVA on ranks and Student-Newman-Keuls post-hoc analysis; *, P<0.05 for Control vs IL-37b transfection; #, P<0.05 for Natural¹⁻²¹⁸ vs D73K¹⁻²¹⁸ or Y85A¹⁻²¹⁸. Dashed line indicates background of IL-1β induction by the IL-37-blocking antibody in Control—(i.e. not IL-37b) transfected cultures.

FIG. 6. Characterization of IL-37-blocking antibody. PBMC from healthy volunteers (n=2) were isolated and pre-treated with various dilutions of the hybridoma supernatants (clone C3 at 1:5 to 1:500) for 30 minutes, followed by treatment with Natural recombinant IL-37 (rIL-37) at various concentrations (from 1 pg/mL to 10 ng/mL) for 1 hour prior to LPS stimulation (500 pg/mL). After an overnight incubation, supernatants were collected and IL-1β was measured by ELISA.

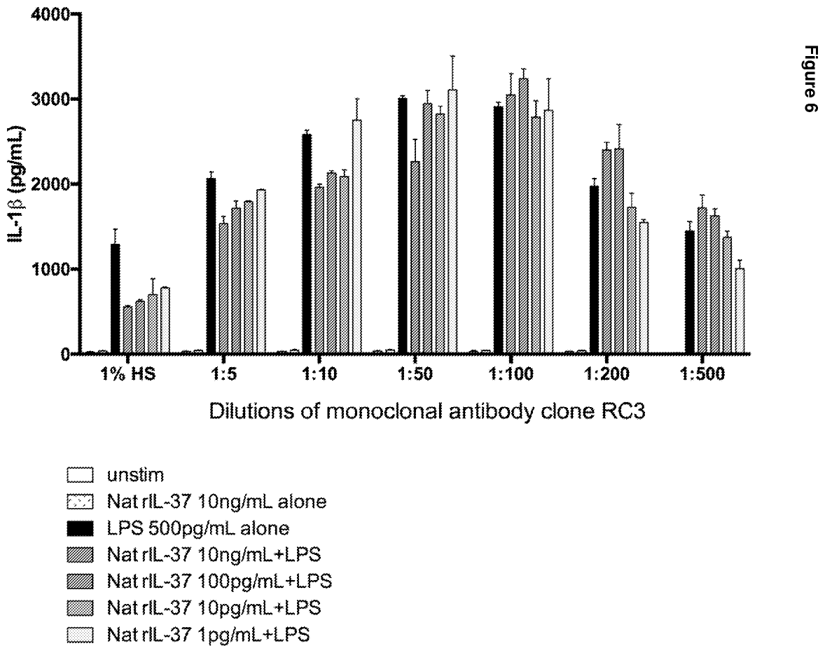

FIG. 7. Blocking of the anti-inflammatory activity of recombinant IL-37 with hybridoma supernatant in human peripheral blood cells. Fresh human PBMCs from two donors were isolated and seeded at 2.5×10⁵ cells/well in a 96-well plate. Cells were allowed to rest for 1 h prior to adding the hybridoma supernatant of clone RD1 at the indicated dilutions. After 30 min incubation, 10 pg/ml recombinant IL-37 (Y85A variant) was added and incubated for 1 h. Cells were then stimulated overnight with 200 pg/ml LPS. IL-1β was measured in the supernatant by ELISA and the change in IL-1β was calculated by comparing to LPS+vehicle or LPS+RD1 as appropriate. Each graph depicts data from one donor as percentage means±s.e.m.

FIG. 8. Blocking of the anti-inflammatory IL-37 activity with hybridoma supernatant in murine splenocytes. Mouse splenocytes were isolated from wild-type (WT) and IL-37 transgenic (IL-37tg) mice (n=2 for WT, n=1 for IL-37tg) and seeded at 2.5×10⁵ cells/well in a 96-well plate. Cells were allowed to rest for 1 h prior to adding the hybridoma clones RF1 (left) and RF3 (right) at the indicated concentrations in biological triplicates. After 30 min incubation, cells were stimulated overnight with LPS (250 ng/mL). IL-6 was measured in the supernatant by ELISA and the change in IL-6 was calculated by comparing LPS+hybridoma supernatant to LPS+vehicle. Data are depicted as percentage means±s.e.m.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed antigen binding sites, for example antibodies, that bind to and inhibit or reduce the activity of IL-37. IL-37 suppresses pro-inflammatory cytokine release in a range of cell types, including human blood cells through anti-inflammatory signalling. The antigen binding sites as described herein have the capacity to inhibit or reduce one or more aspects of the anti-inflammatory activity mediated by IL-37.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-lnterscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

As used herein, reference to IL-37 is to a molecule that has at least one biochemical or biophysical activity of IL-37, for example, it can bind to, and may be a ligand for interleukin 18 receptor alpha (IL-18R1/IL-1Rrp). It may also bind to interleukin 18 binding protein (IL-18BP), an inhibitory binding protein of interleukin 18 (IL-18), and subsequently form a complex with the IL-18 receptor beta chain, and through which it may inhibit the activity of IL-18. Other biochemical or biophysical activities of IL-37 include binding to IL-1R8 (SIGIRR), binding to SMAD3, binding to DNA, blocking the production of pro-, but not anti-inflammatory, cytokines triggered by a broad spectrum of inflammatory assaults including TLR ligands, IFNγ, TNF and IL-1β in human or murine immune cells, inhibition of the activation of dendritic cells (reduction of surface expression of CD86 and MHC II), triggering a specific pattern of regulation of intracellular kinases including blockade of the mTOR, MAPK and NF-κB pathways, and induction of anti-inflammatory kinases such as Mer and PTEN.

IL-37 is also known as interleukin-37 (FIL1 zeta; IL-1 zeta; IL-1F7b (IL-1H4, IL-1H, IL-1RP1); IL-1X protein; IL1F7 (canonical product IL-1F7b); interleukin 1 family member 7; interleukin 1 zeta; interleukin-1 homolog 4; interleukin-1 superfamily z; interleukin-1-related protein). Human IL-37 has 5 isoforms, a, b, c, d and e, all of which are included when referring to IL-37 herein unless expressly stated otherwise. Any isoforms or orthologs of human IL-37 polypeptides are also contemplated within the present invention. For example, the present invention includes polypeptides with identity to any of human IL-37 isoforms a, b, c, d or e.

For the purposes of nomenclature only and without placing any limitation on the scope of the form of IL-37 bound by the antigen binding site of the present invention, an exemplary sequence of a human IL-37 molecule is set out in SEQ ID NO: 1.

The phrase “inhibits IL-37 activity” will be understood to mean that the antigen binding site of the present invention inhibits, antagonises, prevents or reduces any one or more activities of IL-37, including but not limited to, IL-37 binding to IL-18 receptor α (IL-18Rα), to IL-1 receptor 8 or a complex of which comprises IL-18 receptor α (IL-18Rα) and IL-1 receptor 8; IL-37 binding to or interacting with SMAD3; IL-37 binding to or interacting with DNA; IL-37 mediated cell signalling; an IL-37 mediated anti-inflammatory response in vitro or in vivo; IL-37 mediated reduction in cytokine production or secretion; and/or autocrine or paracrine IL-37 mediated reduction in cytokine production or secretion.

Preferably, the cytokine whose production or secretion is reduced by IL-37 is a pro-inflammatory cytokine. Preferably, the cytokine is any one or more described as being regulated by IL-37, including those described in Nold et al. (2010) Nature Immunology 11(11):1014-22, for example, sICAM-1, IFN-gamma, I-TAC, G-SCF, IL-16, IL-10, IL-13, TNF, 1-309, IL-2, IL-7, M-CSF, TIMP-1, IL-1alpha, MIP-1alpha, RANTES, MIG, IL-1Ra, sTREM-1, MCP-5, IP-10, MCP-1, IL-23, KC, IL-1beta, MIP-2, IL-17, eotaxin and IL-6. More preferably, the cytokine is any one or more of M-CSF, TIMP-1, IL-1alpha, MIP-1alpha, RANTES, MIG, IL-1Ra, sTREM-1, MCP-5, IP-10, MCP-1, IL-23, KC, IL-1beta, MIP-2, IL-17, eotaxin and IL-6. Most preferably, the pro-inflammatory cytokine is IL-1β, IL-6 and/or TNF. Examples of cytokines whose production or secretion is reduced by IL-37 and other effects of IL-37 on intracellular pathways are summarised in Nold-Petry et al., (2015) Nature Immunology 16, 354-365. It will be well within the purview of the skilled person to be able to determine which IL-37-mediated pathways can be modified using the antigen-binding sites of the present invention.

Preferably, IL-37 mediated activity is determined using monocytes, macrophages, peripheral blood mononuclear cells (PBMCs), cancer cells or a cell derived therefrom. Preferably, the cancer cell is a lung epithelial cell such as A549, or monocytic leukemia cell such as THP-1. An antigen binding site as described herein may inhibit the effect of IL-37 on any cell type as described herein including monocytes, macrophages, peripheral blood mononuclear cells (PBMCs), cancer cells or a cell derived therefrom.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” is used interchangeably with “antigen binding domain” and shall be taken to mean a region of an antibody that is capable of specifically binding to an antigen, i.e., a V_{H} or a V_{L} or an Fv comprising both a V_{H} and a V_{L}. The antigen binding domain need not be in the context of an entire antibody, e.g., it can be in isolation (e.g., a domain antibody) or in another form, e.g., as described herein, such as a single chain Fv fragment (scFv).

For the purposes for the present disclosure, the term “antibody” includes a protein capable of specifically binding to one or a few closely related antigens (e.g., IL-37) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding site or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding site of the invention reacts or associates more frequently, more rapidly, with greater duration and/or with greater affinity with a particular antigen or cell expressing same than it does with alternative antigens or cells. For example, an antigen binding site binds to IL-37 (e.g., hIL-37) with materially greater affinity (e.g., 1.5 fold or 2 fold or 5 fold or 10 fold or 20 fold or 40 fold or 60 fold or 80 fold to 100 fold or 150 fold or 200 fold) than it does to other interleukins or to antigens commonly recognized by polyreactive natural antibodies (i.e., by naturally occurring antibodies known to bind a variety of antigens naturally found in humans). In an example of the present invention, an antigen binding site that “specifically binds” to hIL-37 with an affinity at least 1.5 fold or 2 fold or greater (e.g., 5 fold or 10 fold or 20 fold r 50 fold or 100 fold or 200 fold) than it does to another interleukin, such as IL-18 and/or Il-1β. Generally, but not necessarily, reference to binding means specific binding, and each term shall be understood to provide explicit support for the other term.

As used herein, the term “does not detectably bind” shall be understood to mean that an antigen binding site, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding may be detected using biosensor analysis (e.g. Biacore) in which the antigen binding site is immobilized and contacted with an antigen.

As used herein, the term “does not significantly bind” shall be understood to mean that the level of binding of an antigen binding site of the invention to a polypeptide is not statistically significantly higher than background, e.g., the level of binding signal detected in the absence of the antigen binding site and/or in the presence of a negative control protein (e.g., an isotype control antibody) and/or the level of binding detected in the presence of a negative control polypeptide. The level of binding may be detected using biosensor analysis (e.g. Biacore) in which the antigen binding site is immobilized and contacted with an antigen.

For the purposes of clarification and as will be apparent to the skilled artisan based on the exemplified subject matter herein, reference to “affinity” in this specification is a reference to KD of a protein or antibody.

For the purposes of clarification and as will be apparent to the skilled artisan based on the description herein, reference to an “affinity of at least about” will be understood to mean that the affinity (or KD) is equal to the recited value or higher (i.e., the value recited as the affinity is lower), i.e., an affinity of 2 nM is greater than an affinity of 3 nM. Stated another way, this term could be “an affinity of X or less”, wherein X is a value recited herein.

As used herein, the term “epitope” (or “antigenic determinant”) shall be understood to mean a region of IL-37 to which an antigen binding site comprising an antigen binding domain of an antibody binds. This term is not necessarily limited to the specific residues or structure to which the antigen binding site makes contact. For example, this term includes the region spanning amino acids contacted by the antigen binding site and 5-10 (or more) or 2-5 or 1-3 amino acids outside of this region. In some examples, the epitope comprises a series of discontinuous amino acids that are positioned close to one another when antigen binding site is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the terms “preventing”, “prevent” or “prevention” include administering an antigen binding site of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding site described herein to thereby reduce or eliminate at least one symptom of a specified disease or condition.

As used herein, the term “subject” shall be taken to mean any animal including humans, for example a mammal. Exemplary subjects include but are not limited to humans and non-human primates, companion animals such as dogs and cats, and farm animals such as horses, sheep and cattle. Preferably, the subject is a human.

Antibodies

In one example, an antigen binding site or IL-37-binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods IL-37 (e.g., hIL-37) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody or a de-immunized antibody.

Antibody Binding Domain Containing Proteins

Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

Other Antibodies and Proteins Comprising Antigen Binding Domains Thereof

The present invention also contemplates other antibodies and proteins comprising antigen-binding domains thereof, such as:

(i) “key and hole” bispecific proteins as described in U.S. Pat. No. 5,731,168;

(ii) heteroconjugate proteins, e.g., as described in U.S. Pat. No. 4,676,980;

(iii) heteroconjugate proteins produced using a chemical cross-linker, e.g., as described in U.S. Pat. No. 4,676,980; and

(iv) Fab₃ (e.g., as described in EP19930302894).

Mutations to Proteins

The present invention also provides an antigen binding site or a nucleic acid encoding same having at least 80% identity to a sequence disclosed herein. In one example, an antigen binding site or nucleic acid of the invention comprises sequence at least about 85% or 90% or 92% or 95% or 97% or 98% or 99% identical to a sequence disclosed herein.

Alternatively, or additionally, the antigen binding site comprises a CDR (e.g., three CDRs) at least about 80% or 85% or 90% or 92% or 95% or 97% or 98% or 99% identical to CDR(s) of a V_{H} or V_{L} as described herein according to any example.

In another example, a nucleic acid of the invention comprises a sequence at least about 80% or 85% or 90% or 92% or 95% or 97% or 98% or 99% identical to a sequence encoding an antigen binding site having a function as described herein according to any example. The present invention also encompasses nucleic acids encoding an antigen binding site of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding site of the invention comprising one or more conservative amino acid substitutions compared to a sequence set forth herein. In some examples, the antigen binding site comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding site comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding site of the invention. In another example, the mutation(s) occur within a CDR of an antigen binding site of the invention.

Exemplary methods for producing mutant forms of an antigen binding site include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1 Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding sites of the invention will be apparent to the skilled artisan and/or described herein, e.g., antigen binding. For example, methods for determining antigen binding, competitive inhibition of binding, affinity, association, dissociation and therapeutic efficacy are described herein.

Constant Regions

The present invention encompasses antigen binding sites and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding site comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

Protein Production

In one example, an antigen binding site described herein according to any example is produced by culturing a hybridoma under conditions sufficient to produce the protein, e.g., as described herein and/or as is known in the art.

Recombinant Expression

In another example, an antigen binding site described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Isolation of Proteins

Methods for isolating a protein are known in the art and/or described herein.

Where an antigen binding site is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding site prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying Activity of an IL-37-Binding Protein

Binding to IL-37 and Mutants Thereof

It will be apparent to the skilled artisan from the disclosure herein that some antigen binding sites of the present invention bind to IL-37 and to specific mutant forms of IL-37. Methods for assessing binding to a protein are known in the art, e.g., as described in Scopes (In: Protein purification: principles and practice, Third Edition, Springer Verlag, 1994). Such a method generally involves immobilizing the antigen binding site and contacting it with labelled antigen. Following washing to remove non-specific bound protein, the amount of label and, as a consequence, bound antigen is detected. Of course, the antigen binding site can be labelled and the antigen immobilized. Panning-type assays can also be used. Alternatively, or additionally, surface plasmon resonance assays can be used.

Optionally, the dissociation constant (Kd), association constant (Ka) and/or affinity constant (K_{D}) of an immobilized antigen binding site for IL-37 or an epitope thereof is determined. The “Kd” or “Ka” or “K_{D}” for an IL-37-binding protein is in one example measured by a radiolabelled or fluorescently-labelled IL-37 binding assay. In the case of a “Kd”, this assay equilibrates the antigen binding site with a minimal concentration of labelled IL-37 in the presence of a titration series of unlabelled IL-37. Following washing to remove unbound IL-37, the amount of label is determined, which is indicative of the Kd of the protein.

According to another example the Kd, Ka or K_{D} is measured by using surface plasmon resonance assays, e.g., using BIAcore surface plasmon resonance (BIAcore, Inc., Piscataway, N.J.) with immobilized IL-37 or a region thereof or immobilized antigen binding site.

Determining Inhibitory Activity

In some examples of the present invention, an antigen binding site is capable of inhibiting IL-37 activity.

Various assays are known in the art for assessing the ability of a protein to inhibit or reduce signalling of a ligand through a receptor.

In one example, the antigen binding site promotes production or secretion of pro-inflammatory cytokines such as IL-1β in cells (e.g., PBMC) expressing IL-18 receptor α (IL-18Rα) and IL-1 receptor 8 (or alternatively, cells modified to express both proteins) which are cultured in the presence of recIL-37 (recombinant IL-37). Cells (e.g., about 1×10⁴ cells) are cultured in the presence of recIL-37 (e.g., between about 1 pg/mL to about 10 ng/mL (such as 1 pg/mL or 10 ng/mL) and the presence or absence of a test antigen binding site, or a pro-inflammatory stimulus and an antigen binding site. Methods for assessing production or secretion of pro-inflammatory cytokines are known in the art. An antigen binding site that enhances the level of production or secretion of pro-inflammatory cytokines compared to the level observed in the absence of the antigen binding site is considered to inhibit or reduce IL-37 activity, specifically IL-37 signalling.

Other methods for assessing enhancement of IL-37 signalling are contemplated by the present disclosure including THP-1 cell assays as described in Example 6.

Assessing Therapeutic Efficacy

Assays for assessing therapeutic efficacy are described hereinabove in relation to determining neutralization by an antigen binding site.

In another example, the efficacy of a protein to treat a condition is assessed using an in vivo assay.

For example, the antigen binding site can be tested in a model of cancer, e.g., gastric cancer. For example, mice homologous for the Y757F mutant of gp130 (gp130^{Y757F/Y757F}) develop gastric tumors Jenkins et al, Blood 109: 2380-2388, 2007). Mice (e.g., eight week old mice) are treated with an antigen binding site and the number and/or weight of gastric polyps assessed. An antigen binding site that reduces polyp size and/or weight is considered useful for treating cancer. A similar assay can be used to test for an effect on colon cancer, in which gp130^{Y757F/Y757F} mice are treated with azoxymethane (AOM) followed by dextran sodium sulfate (DSS) essentially as described in Greten et al, Cell, 118: 285-296, 2004 to induce colon cancer prior to treatment with the antigen binding site.

The antigen binding site can additionally or alternatively be tested in a model of cancer metastasis or cancer-related bone disease, e.g., as described in Li et al., Oncol. Lett. 3: 802-806, 2012.

Conditions to be Treated or Prevented

The antigen binding sites of the present invention are useful in the treatment or prevention of a condition associated with expression of endogenous IL-37, including “normal” levels of expression or elevated expression. Elevated expression of endogenous IL-37 may be determined by measuring the amount or concentration of IL-37 in the circulation of a subject or at a particular location (e.g. in a specific tissue or organ) in the subject. The expression of IL-37 may be determined by measuring mRNA or protein levels using techniques known to those skilled in the art.

Further, the antigen binding sites of the present invention are useful in the treatment or prevention of cancers. Broad examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bron chogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestationaltrophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, Ihfraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed cell, null-cell, t-cell, t-cell chronic, htlv-ii-associated, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplasia syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ovarian cancer, pancreas cancer, paranasal cancer, parathyroid cancer, parotid gland cancer, penile cancer, peripheral-.neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), Sertoli cell tumor, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

The present invention also provides a method for treating or preventing a condition associated with immune paralysis in a subject, the method comprising administering an antigen binding site of the invention to the subject, thereby treating or preventing a condition associated with immune paralysis. As used herein, the term “immune paralysis” refers to any condition characterised by reduced responsiveness or non-responsiveness of the immune system of a subject. Immune paralysis may follow a period of hyper-immune response (including an intense surge in cytokine production) in response to sepsis or other infection or disease. Immune paralysis may be characterised by apoptosis of immune cells and/or high levels of anti-inflammatory cytokines that inhibit lymphocytes and macrophages and suppress the production of pro-inflammatory cytokines. Exemplary conditions associated with immune paralysis include sepsis, and acute and chronic liver failure.

Use as a Diagnostic/Analytical Reagent

It will be appreciated that the antigen binding sites of the present invention (including antibodies comprising the antigen binding sites, or comprising a CDR or FR sequence as described herein) also have utility in determining the levels of IL-37 in a biological sample.

For example, the binding of the antigen binding sites of the invention to IL-37 (as described in further detail above) can be used as a means for quantitating or evaluating the levels of IL-37 in a biological sample obtained from an individual. The biological sample may include any sample obtained from the individual for whom diagnosis is to be determined, including a blood sample (which may be further processed to PBMCs, serum or plasma), saliva, sweat, a tissue biopsy and the like.

As such, in one embodiment, the antigen binding sites may be useful as diagnostic reagents, to diagnose any one or more conditions associated with increased or decreased levels of IL-37, as the case may be. The antigen binding sites may also be used to monitor the progress of treatment for a condition associated with altered levels of IL-37 (for example, the antigen binding sites can be used to determine whether the levels of IL-37 in the individual are altered in response to any intervention used to treat or ameliorate the condition that is characterised by abnormal IL-37 levels).

The individual requiring diagnosis may be any individual suspected of having, or being at risk of a condition characterised by altered levels of IL-37 production or secretion. The altered levels may be increased or decreased levels in comparison to what would otherwise be expected for the individual. Depending on the particular circumstance, the levels of IL-37 are compared to those that are known for a reference data set. The reference data set may correspond to the levels of IL-37 in individuals who do or do not have the condition for which the individual is being diagnosed. Alternatively, the reference data set may be data obtained from the individual at an earlier time point.

In alternative embodiments, the antigen binding sites may be used as analytical reagents, to determine IL-37 levels in samples obtained during in vitro or in vivo experimentation. For example, the antigen binding sites may be used to quantify levels of IL-37 in supernatants from tissue cultures.

The skilled person will be familiar with methods for adapting the antigen binding sites described herein for use as diagnostic or analytic reagents. For example, the antigen binding sites may be adapted for use in an enzyme-linked immunosorbent assay (ELISA), including but not limited to indirect ELISA, sandwich ELISA, competitive ELISA, multiple and portable ELISA; and radioimmunoassay (RIA) and the like.

Compositions

In some examples, an antigen binding site as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding site into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding site of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

Dosages and Timing of Administration

Suitable dosages of an antigen binding site of the present invention will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

Kits

The present invention additionally comprises a kit comprising one or more of the following:

(i) an antigen binding site of the invention or expression construct(s) encoding same;

(ii) a cell of the invention;

(iii) a complex of the invention; or

(iii) a pharmaceutical composition of the invention.

In the case of a kit for detecting IL-37, the kit can additionally comprise a detection means, e.g., linked to an antigen binding site of the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Light chain and heavy chain CDR sequences

| Antibody | | | | | | |
|---|---|---|---|---|---|---|
| ID | LCDR1 | LCDR2 | LCDR3 | HCDR1 | HCDR2 | HCDR3 |
| Antibody | SASSSVSYMH | DTSKLAS | QQWSSNPPT | SYGVH | VIWRGGSTDYNAAF | ELWLRRGFAY |
| C3 | (SEQ ID NO: 2) | (SEQ ID | (SEQ ID | (SED ID | MS | (SEQ ID |
| | | NO: 3) | NO: 4) | NO: 5) | (SEQ ID NO: 6) | NO: 7) |
| Antibody | KSSQSLLYSSNQKNY | WASTRES | QQYYSYPFT | DTYMH | RIDPANGNTKYDPKF | SSSPTWFAY |
| D3 | LA | (SEQ ID | (SEQ ID | (SEQ ID | QG | (SEQ ID |
| | (SEQ ID NO: 12) | NO: 13) | NO: 14) | NO: 15) | (SEQ ID NO: 16) | NO: 17) |
| Antibody | RASESVDIYGNSFMH | RASNLES | QQSYEDPWT | NYGMN | WINTYIGEPTYADDFK | NYGYPWFAY |
| RD1 (A) | (SEQ ID NO: 38) | (SEQ ID | (SEQ ID | (SEQ ID | G | (SEQ ID |
| | | NO: 39) | NO: 40) | NO: 41) | (SEQ ID NO: 42) | NO: 43) |
| Antibody | RASESVDIYGNSFMH | RASNLES | QQSYEDPWT | DYSMH | WINTETGEPTYADDF | SYAYAMDY |
| RD1 (B) | (SEQ ID NO: 48) | (SEQ ID | (SEQ ID | (SEQ ID | KG | (SEQ ID |
| | | NO: 49) | NO: 50) | NO: 51) | (SEQ ID NO: 52) | NO: 53) |
| Antibody | KASQNVGTNVA | SASYRYS | QQYNSYPLT | NYGMN | WINTYIGEPTYADDFK | NYGYPWFAY |
| RD1 (C) | (SEQ ID NO: 58) | (SEQ ID | (SEQ ID | (SEQ ID | G | (SEQ ID |
| | | NO: 59) | NO: 60) | NO: 61) | (SEQ ID NO: 62) | NO: 63) |
| Antibody | KASQNVGTNVA | SASYRYS | QQYNSYPLT | DYSMH | WINTETGEPTYADDF | SYAYAMDY |
| RD1 (D) | (SEQ ID NO: 68) | (SEQ ID | (SEQ ID | (SEQ ID | KG | (SEQ ID |
| | | NO: 69) | NO: 70) | NO: 71) | (SEQ ID NO: 72) | NO: 73) |
| Antibody | RSSQSLVHSNGNTYL | KVSNRFS | SQCRHVPFT | DTYMH | RIDPANGNSIYDPTFQ | GNWDWFVF |
| RE1 | H | (SEQ ID | (SEQ ID | (SEQ ID | G | (SEQ ID |
| | (SEQ ID NO: 78) | NO: 79) | NO: 80) | NO: 81) | (SEQ ID NO: 82) | NO: 83) |
| Antibody | KASQNVDTHVA | SASYRYS | QQYHSYPLT | KKGVT | IIWDDGSINYHSALIS | RGDVSRGAWFVY |
| RB6 | (SEQ ID NO: 88) | (SEQ ID | (SEQ ID | (SEQ ID | (SEQ ID NO: 92) | (SEQ ID |
| | | NO: 89) | NO: 90) | NO: 91) | | NO: 93) |
| Antibody | RASESVDIYGNSFMH | RASNLES | QQSYEDPWT | DYSMH | WINTETGEPTYADDF | SYAYAMDY |
| RH1 (A) | (SEQ ID NO: 98) | (SEQ ID | (SEQ ID | (SEQ ID | KG | (SEQ ID |
| | | NO: 99) | NO: 100) | NO: 101) | (SEQ ID NO: 102) | NO: 103) |
| Antibody | RASESVDIYGNSFMH | RASNLES | QQSYEDPWT | NYGMN | WINTFTGEPSYADDF | NYGYPWFAY |
| RH1 (B) | (SEQ ID NO: 108) | (SEQ ID | (SEQ ID | (SEQ ID | KG | (SEQ ID |
| | | NO: 109) | NO: 110) | NO: 111) | (SEQ ID NO: 112) | NO: 113) |
| Antibody | RASKSVSTSGYSYM | LVSNLES | QQSYEDPWT | DYSMH | WINTETGEPTYADDF | SYAYAMDY |
| RH1 (C) | H | (SEQ ID | (SEQ ID | (SEQ ID | KG | (SEQ ID |
| | (SEQ ID NO: 118) | NO: 119) | NO: 120) | NO: 121) | (SEQ ID NO: 122) | NO: 123) |
| Antibody | RASKSVSTSGYSYM | LVSNLES | QQSYEDPWT | NYGMN | WINTFTGEPSYADDF | NYGYPWFAY |
| RH1 (D) | H | (SEQ ID | (SEQ ID | (SEQ ID | KG | (SEQ ID |
| | (SEQ ID NO: 128) | NO: 129) | NO: 130) | NO: 131) | (SEQ ID NO: 132) | NO: 133) |
| Antibody | RSSQSLVHSNGDSYL | KVSNRLS | SQSTHVPYT | NYWLH | MIDPSDSFTTYNQKF | VDH |
| RF1 | H | (SEQ ID | (SEQ ID | (SEQ ID | KG | (SEQ ID |
| | (SEQ ID NO: 138) | NO: 139) | NO: 140) | NO: 141) | (SEQ ID NO: 142) | NO: 143) |
| Antibody | RSSQSLVHSNGNTYL | IVSNRFS | SQSTHIPYT | NYWMN | MIDPSDSETHYNQVF | DFG |
| RF3 | H | (SEQ ID | (SEQ ID | (SEQ ID | KD | (SEQ ID |
| | (SEQ ID NO: 148) | NO: 149) | NO: 150) | NO: 151) | (SEQ ID NO: 152) | NO: 153) |
| Antibody | RSSQSIVHSNGNTYL | KVSNRFS | FQGSHVPWT | NYLIE | VINPGSGGTNYNEKF | DDSAY |
| R45 (A) | E | (SEQ ID | (SEQ ID | (SEQ ID | KG | (SEQ ID |
| | (SEQ ID NO: 158) | NO: 159) | NO: 160) | NO: 161) | (SEQ ID NO: 162) | NO: 163) |
| Antibody | RASQSISNNLH | YASQSIS | QQSNSWPLT | NYLIE | VINPGSGGTNYNEKF | DDSAY |
| RA5 (B) | (SEQ ID NO: 168) | (SEQ ID | (SEQ ID | (SEQ ID | KG | (SEQ ID |
| | | NO: 169) | NO: 170) | NO: 171) | (SEQ ID NO: 172) | NO: 173) |
| Antibody | RSSQSIVHSNGNTYL | KVSNRFS | FQGSHVPWT | SYWMH | EIDPSDSYTNYNQKF | MITTQGYFDY |
| RD3 | E | (SEQ ID | (SEQ ID | (SEQ ID | KG | (SEQ ID |
| | (SEQ ID NO: 178) | NO: 179) | NO: 180) | NO: 181) | (SEQ ID NO: 182) | NO: 183) |
| Antibody | RSSLSLVYSNGNTYL | KVSNRFS | SQNTHVPFT | DTYIH | RIDPANGNTKYDPKF | GNWDWFAY |
| RC1 | H | (SEQ ID | (SEQ ID | (SEQ ID | QD | (SEQ ID |
| | (SEQ ID NO: 188) | NO: 189) | NO: 190) | NO: 191) | (SEQ ID NO: 192) | NO: 193) |
| Antibody | KSGQSLVHSNGNTYL | KVSNRFS | SQSTHVPLT | DYYMH | WIDPENGNTIYDPKF | EDY |
| RF2 | R | (SEQ ID | (SEQ ID | (SEQ ID | QG | (SEQ ID |
| | (SEQ ID NO: 198) | NO: 199) | NO: 200) | NO: 201) | (SEQ ID NO: 202) | NO: 203) |

TABLE 2
Light chain and heavy chain variable region sequences

| | VL (FR1-CDR1- | VH (FR1-CDR1- | | |
|---|---|---|---|---|
| Anti- | FR2-CDR2-FR3- | FR2-CDR2-FR3- | | |
| body | CDR3-FR4) | CDR3-FR4) | | |
| ID | (amino acid) | (amino acid) | VL (nucleotide) | VH (nucleotide) |
| Anti- | QIVLTQSPAIMSASP | QVQLKQSGPGLVQ | CAAATTGTTCTCACCCAGTCTCCAGCAATCATGTC | CAGGTGCAGCTGAAGCAGTCAGGACCTGGCCTA |
| body | GEKVTMTCSASSS | PSQSLSITCTVSGF | TGCATCTCCAGGGGAGAAGGTCACCATGACCTGC | GTGCAGCCCTCACAGAGCCTGTCCATAACCTGCA |
| C3 | VSYMHWYQQKSGT | SLTSYGVHWVRQS | AGTGCCAGCTCAAGTGTAAGTTACATGCACTGGT | CAGTCTCTGGTTTCTCATTAACTAGCTATGGTGTA |
| | SPKRWYDTSKLAS | PGKGLEWLGVIWR | ACCAGCAGAAGTCAGGCACCTCCCCCAAAAGATG | CACTGGGTTCGCCAGTCTCCAGGAAAGGGTCTG |
| | GVPARFSGSGSGT | GGSTDYNAAFMSR | GATTTATGACACATCCAAACTGGCTTCTGGAGTCC | GAGTGGCTGGGAGTGATATGGAGAGGTGGAAGC |
| | SYSLTISSMEAEDA | LSITKDNSKSQVFF | CTGCTCGCTTCAGTGGCAGTGGGTCTGGGACCTC | ACAGACTACAATGCAGCTTTCATGTCCAGACTGA |
| | ATYYCQQWSSNPP | KMNSLQADDTAIYY | TTACTCTCTCACAATCAGCAGCATGGAGGCTGAA | GCATCACCAAGGACAACTCCAAGAGCCAAGTTTT |
| | TFGGGTKLEIK | CAKELWLRRGFAY | GATGCTGCCACTTATTACTGCCAGCAGTGGAGTA | CTTTAAAATGAACAGTCTGCAAGCTGATGACACTG |
| | (SEQ ID | WGQGTLVTVSA | GTAACCCACCGACGTTCGGTGGAGGCACCAAGC | CCATATACTACTGTGCCAAGGAGCTATGGTTACG |
| | NO: 8) | (SEQ ID | TGGAAATCAAA (SEQ ID NO: 10) | ACGAGGGTTTGCTTACTGGGGCCAAGGGACTCTG |
| | | NO: 9) | | GTCACTGTCTCTGCA (SEQ ID NO: 11) |
| Anti- | DIVMSQSPSSLAVS | EVQLQQSGAELVK | CTGTGTCAGTTGGAGAGAAGGTTACTATGAGCTG | GAGGTTCAGCTGCAGCAGTCTGGGGCAGAGCTT |
| body | VGEKVTMSCKSSQ | PGASVKLSCTASGF | GAGATTGTGATGTCACAGTCTCCATCCTCCCTAG | GTGAAGCCAGGGGCCTCAGTCAAGTTGTCCTGCA |
| D3 | SLLYSSNQKNYLAW | NIKDTYMHWVKQR | CAAGTCCAGTCAGAGCCTTTTATATAGTAGCAATC | CAGCTTCTGGCTTCAACATTAAAGACACCTATATG |
| | YQQKPGQSPKLLIY | PEQGLEWIGRIDPA | AAAAAGAACTACTTGGCCTGGTACCAGCAGAAACC | CACTGGGTGAAGCAGAGGCCTGAACAGGGCCTG |
| | WASTRESGVPDRF | NGNTKYDPKFQGK | AGGGCAGTCTCCTAAACTGCTGATTTACTGGGCA | GAGTGGATTGGAAGGATTGATCCTGCGAATGGTA |
| | TGSGSGTDFTLTIS | ATITADTSSNTAYLQ | TCCACTAGGGAATCTGGGGTCCCTGATCGCTTCA | ATACTAAATATGACCCGAAGTTCCAGGGCAAGGC |
| | SVKAEDLAVYYCQ | LSSLTSEDTAVYYC | CAGGCAGTGGATCTGGGACAGATTTCACTCTCAC | CACTATAACAGCAGACACATCCTCCAACACAGCC |
| | QYYSYPFTFGSGTK | AKSSSPTWFAYWG | CATCAGCAGTGTGAAGGCTGAAGACCTGGCAGTT | TACCTGCAGCTCAGCAGCCTGACATCTGAGGAGA |
| | LEIK | QGTLVTVSA | TATTACTGTCAGCAATATTATAGCTATCCATTCAC | CTGCCGTCTATTACTGTGCTAAGAGTAGTAGCCC |
| | (SEQ ID | (SEQ ID | GTTCGGCTCGGGGACAAAGTTGGAAATAAAA | CACCTGGTTTGCTTACTGGGGCCAAGGGACTCTG |
| | NO: 18) | NO: 19) | (SEQ ID NO: 20) | GTCACTGTCTCTGCA (SEQ ID NO: 21) |
| Anti- | DIVLTQSPASLAVSL | QIQLVQSGPELKKP | GACATTGTGCTGACCCAATCTCCAGCTTCTTTGG | CAGATCCAGTTGGTGCAGTCTGGACCTGAGCTGA |
| body | GQRAIISCRASESV | GETVKISCKASGYT | CTGTGTCTCTAGGGCAGAGGGCCATCATATCCTG | AGAAGCCTGGAGAGACAGTCAAGATCTCCTGCAA |
| RD1 | DIYGNSFMHWCQQ | FKNYGMNWVKQAP | CAGAGCCAGTGAAAGTGTTGATATTTATGGCAATA | GGCTTCTGGGTATACCTTCAAAAACTATGGAATGA |
| (A) | KPGQPPKLLIFRAS | GKGLKWMGWINTYI | GTTTATGCACTGGTGCCAGCAGAAGCCAGGACA | ACTGGGTGAAGCAGGCTCCAGGAAAGGGTTTAAA |
| | NLESGIPARFSGSG | GEPTYADDFKGRF | GCCACCCAAACTCCTCATCTTTCGTGCATCCAAC | GTGGATGGGCTGGATAAACACCTATATTGGAGAG |
| | SRTDFTLTINPVEAD | AFSLETSASTAYLQI | CTAGAATCTGGGATCCCTGCCAGGTTCAGTGGCA | CCAACATATGCTGATGACTTCAAGGGACGGTTTG |
| | DVATYYCQQSYED | NNLKDEDTATYFCA | GTGGGTCTAGGACAGACTTCACCCTCACCATTAA | CCTTCTCTTTGGAAACCTCTGCCAGCACTGCCTAT |
| | PWTFGGGTKLEIK | RNYGYPWFAYWG | TCCTGTGGAGGCTGATGATGTTGCAACCTATTACT | TTGCAGATCAACAACCTCAAAGATGAGGACACGG |
| | (SEQ ID | QGTLVTVSA | GTCAACAAAGTTATGAGGATCCGTGGACGTTCGG | CTACATATTTCTGTGCAAGAAACTACGGCTACCCC |
| | NO: 44) | (SEQ ID | TGGAGGCACCAAGCTGGAAATCAAA | TGGTTTGCTTACTGGGGCCAAGGGACTCTGGTCA |
| | | NO: 45) | (SEQ ID NO: 46) | CTGTCTCTGCA (SEQ ID NO: 47) |
| Anti- | DIVLIQSPASLAVSL | QIQLVQSGPELEKP | GACATTGTGCTGACCCAATCTCCAGCTTCTTTGG | CAGATCCAGTTGGTGCAGTCTGGACCTGAGCTGG |
| body | GQRAIISCRASESV | GETVKISCKASGYT | CTGTGTCTCTAGGGCAGAGGGCCATCATATCCTG | AGAAGCCTGGAGAGACAGTCAAGATCTCCTGTAA |
| RD1 | DIYGNSFMHWCQQ | FTDYSMHWVKQAP | CAGAGCCAGTGAAAGTGTTGATATTTATGGCAATA | GGCTTCTGGTTATACCTTCACAGACTATTCAATGC |
| (B) | KPGQPPKLLIFRAS | GKGLKWMGWINTE | GTTTTATGCACTGGTGCCAGCAGAAGCCAGGACA | ACTGGGTGAAGCAGGCTCCAGGAAAGGGTTTAAA |
| | NLESGIPARFSGSG | TGEPTYADDFKGRF | GCCACCCAAACTCCTCATCTTTCGTGCATCCAAC | GTGGATGGGCTGGATAAACACTGAGACTGGTGAG |
| | SRTDFTLTINPVEAD | AFSLETSASTAYLQI | CTAGAATCTGGGATCCCTGCCAGGTTCAGTGGCA | CCAACATATGCAGATGACTTCAAGGGACGGTTTG |
| | DVATYYCQQSYED | NNLKNEDTATYFCA | GTGGGTCTAGGACAGACTTCACCCTCACCATTAA | CCTTCTCGTTGGAAACCTCTGCCAGCACTGCCTA |
| | PWTFGGGTKLEIK | RSYAYAMDYWGQ | TCCTGTGGAGGCTGATGATGTTGCAACCTATTACT | TTTGCAGATCAACAACCTCAAAAATGAGGACACG |
| | (SEQ ID | GTSVTVSS | GTCAACAAAGTTATGAGGATCCGTGGACGTTCGG | GCTACATATTTCTGTGCTAGATCTTACGCCTATGC |
| | NO: 54) | (SEQ ID | TGGAGGCACCAAGCTGGAAATCAAA | TATGGACTACTGGGGTCAAGGAACCTCAGTCACC |
| | | NO: 55) | (SEQ ID NO: 56) | GTCTCCTCA (SEQ ID NO: 57) |
| Anti- | DIVMTQSQKFMSTS | QIQLVQSGPELKKP | GACATTGTCATGACCCAGTCTCAAAAATTCATGTC | CAGATCCAGTTGGTGCAGTCTGGACCTGAGCTGA |
| body | VGDRVSVTCKASQ | GETVKISCKASGYT | CACATCAGTAGGAGACAGGGTCAGCGTCACCTGC | AGAAGCCTGGAGAGACAGTCAAGATCTCCTGCAA |
| RD1 | NVGTNVAWYQQKP | FKNYGMNWVKQAP | AAGGCCAGTCAGAATGTGGGTACTAATGTAGCCT | GGCTTCTGGGTATACCTTCAAAAACTATGGAATGA |
| (C) | GQSPKALIYSASYR | GKGLKWMGWINTYI | GGTATCAACAGAAACCAGGGCAATCTCCTAAAGC | ACTGGGTGAAGCAGGCTCCAGGAAAGGGTTTAAA |
| | YSGVPDRFTGSGS | GEPTYADDFKGRF | ACTGATTTACTCGGCATCCTACCGGTACAGTGGA | GTGGATGGGCTGGATAAACACCTATATTGGAGAG |
| | GTDFTLTISNVQSE | AFSLETSASTAYLQI | GTCCCTGATCGCTTCACAGGCAGTGGATCTGGGA | CCAACATATGCTGATGACTTCAAGGGACGGTTTG |
| | DLAEYFCQQYNSY | NNLKDEDTATYFCA | CAGATTTCACTCTCACCATCAGCAATGTGCAGTCT | CCTTCTCTTTGGAAACCTCTGCCAGCACTGCCTAT |
| | PLTFGAGTKLELK | RNYGYPWFAYWG | GAAGACTTGGCAGAGTATTTCTGTCAGCAATATAA | TTGCAGATCAACAACCTCAAAGATGAGGACACGG |
| | (SEQ ID | QGTLVTVSA | CAGCTATCCGCTCACGTTCGGTGCTGGGACCAAG | CTACATATTTCTGTGCAAGAAACTACGGCTACCCC |
| | NO: 64) | (SEQ ID | CTGGAGCTGAAA | TGGTTTGCTTACTGGGGCCAAGGGACTCTGGTCA |
| | | NO: 65) | (SEQ ID NO: 66) | CTGTCTCTGCA (SEQ ID NO: 67) |
| Anti- | DIVMTQSQKFMSTS | QIQLVQSGPELEKP | GACATTGTCATGACCCAGTCTCAAAAATTCATGTC | CAGATCCAGTTGGTGCAGTCTGGACCTGAGCTGG |
| body | VGDRVSVTCKASQ | GETVKISCKASGYT | CACATCAGTAGGAGACAGGGTCAGCGTCACCTGC | AGAAGCCTGGAGAGACAGTCAAGATCTCCTGTAA |
| RD1 | NVGTNVAWYQQKP | FTDYSMHWVKQAP | AAGGCCAGTCAGAATGTGGGTACTAATGTAGCCT | GGCTTCTGGTTATACCTTCACAGACTATTCAATGC |
| (D) | GQSPKALIYSASYR | GKGLKWMGWINTE | GGTATCAACAGAAACCAGGGCAATCTCCTAAAGC | ACTGGGTGAAGCAGGCTCCAGGAAAGGGTTTAAA |
| | YSGVPDRFTGSGS | TGEPTYADDFKGRF | ACTGATTTACTCGGCATCCTACCGGTACAGTGGA | GTGGATGGGCTGGATAAACACTGAGACTGGTGAG |
| | GTDFTLTISNVQSE | AFSLETSASTAYLQI | GTCCCTGATCGCTTCACAGGCAGTGGATCTGGGA | CCAACATATGCAGATGACTTCAAGGGACGGTTTG |
| | DLAEYFCQQYNSY | NNLKNEDTATYFCA | CAGATTTCACTCTCACCATCAGCAATGTGCAGTCT | CCTTCTCGTTGGAAACCTCTGCCAGCACTGCCTA |
| | PLTFGAGTKLELK | RSYKYAMDYWGQ | GAAGACTTGGCAGAGTATTTCTGTCAGCAATATAA | TTTGCAGATCAACAACCTCAAAAATGAGGACACG |
| | (SEQ ID | GTSVTVSS | CAGCTATCCGCTCACGTTCGGTGCTGGGACCAAG | GCTACATATTTCTGTGCTAGATCTTACGCCTATGC |
| | NO: 74) | (SEQ ID | CTGGAGCTGAAA | TATGGACTACTGGGGTCAAGGAACCTCAGTCACC |
| | | NO: 75) | (SEQ ID NO: 76) | GTCTCCTCA (SEQ ID NO: 77) |
| Anti- | DVVMTQTPLSLPVS | EVQLQQSGAELVK | GATGTTGTGATGACCCAAACTCCACTCTCCCTGC | GAGGTTCAGCTGCAGCAGTCTGGGGCAGAACTT |
| body | LGDQASISCRSSQS | PGASVTLSCTASGF | CTGTCAGTCTTGGAGATCAAGCCTCCATCTCTTG | GTGAAGCCAGGGGCCTCAGTCACGTTGTCCTGCA |
| RE1 | LVHSNGNTYLHWY | NIKDTYMHWVKQR | CAGATCTAGTCAGAGCCTTGTACACAGTAATGGA | CAGCTTCTGGCTTCAACATTAAAGACACCTATATG |
| | LQKPGQSPKLLIYK | PEQGLEWIGRIDPA | AACACCTATTTACATTGGTACCTGCAGAAGCCAG | CACTGGGTGAAGCAGAGGCCTGAACAGGGCCTG |
| | VSNRFSGVPDRFS | NGNSIYDPTFQGKA | GCCAGTCTCCAAAGCTCCTGATCTACAAAGTTTCC | GAGTGGATTGGAAGGATTGATCCTGCGAATGGTA |
| | GSGSGTDFTLKISR | TVTADTSSNTAYLQ | AACCGATTTTCTGGGGTCCCAGACAGGTTCAGTG | ATTCTATATATGACCCGACGTTCCAGGGCAAGCC |
| | VEAEDLGVYFCSQ | LSSLTSEDTAVYFC | GCAGTGGATCAGGGACAGATTTCACACTCAAGAT | CACTGTAACAGCTGACACATCCTCCAACACAGCC |
| | CRHVPFTFGSGTKL | SNGNWDWFVFWG | CAGCAGAGTGGAGGCTGAGGATCTGGGAGTTTAT | TACCTGCAGCTCAGCAGCCTGACATCTGAGGACA |
| | EIK | QGTLVTVSA | TTCTGCTCTCAATGTAGACATGTTCCATTCACGTT | CTGCCGTCTATTTCTGTTCTAATGGGAACTGGGA |
| | (SEQ ID | (SEQ ID | CGGCTCGGGGACAAAGTTGGAAATAAAA | CTGGTTTGTTTTCTGGGGCCAAGGGACTCTGGTC |
| | NO: 84) | NO: 85) | (SEQ ID NO: 86) | ACTGTCTCTGCA (SEQ ID NO: 87) |
| Anti- | DIVMTQSQEFLSTL | QVQLKESGPGLVA | GACATTGTGATGACCCAGTCTCAAGAATTCTTGTC | CAGGTGCAGTTGAAGGAGTCAGGACCTGGCCTG |
| body | VGDRVSVTCKASQ | PSQSLTITCTVSGF | CACATTAGTAGGAGACAGGGTCAGCGTCACCTGC | GTGGCGCCCTCACAGAGCCTGACCATCACATGCA |
| RB6 | NVDTHVAWYQQKP | SLTKKGVTWVRQP | AAGGCCAGTCAGAATGTGGATACTCATGTAGCCT | CTGTCTCAGGGTTCTCCTTAACCAAAAAAGGTGTA |
| | GQSPKTLIYSASYR | PGKGLEWLGIIWDD | GGTATCAACAGAAACCAGGGCAGTCTCCTAAAAC | ACGTGGGTTCGCCAGCCTCCAGGAAAGGGTCTG |
| | YSGVPDRFTGSGS | GSINYHSALISRLRI | ACTGATTTACTCGGCATCTTACCGGTACAGTGGA | GAGTGGCTGGGAATAATATGGGATGACGGGAGC |
| | GTDFTLTISSVQSE | TKDNFKSQVFLHLN | GTCCCTGATCGCTTCACAGGCAGTGGATCTGGGA | ATTAATTATCACTCAGCTCTCATATCCAGATTGCG |
| | DLAKYFCQQYHSY | SLQTDDTATYYCAR | CAGATTTCACTCTCACCATCAGCAGTGTGCAGTCT | CATCACTAAGGATAATTTCAAGAGCCAAGTTTTCC |
| | PLTFGGGTKLELK | RGDVSRGAWFVY | GAAGACTTGGCAAAGTATTTCTGTCAACAATATCA | TGCACCTGAACAGTCTGCAAACTGATGATACAGC |
| | (SEQ ID | WGQGTLVTVSA | CAGCTATCCTCTCACGTTCGGAGGGGGGACCAA | CACGTATTACTGTGCCAGAAGAGGGGACGTTAGT |
| | NO: 94) | (SEQ ID | GCTGGAACTGAAA | AGGGGGGCCTGGTTTGTCTATTGGGGCCAAGGG |
| | | NO: 95) | (SEQ ID NO: 96) | ACTCTGGTCACTGTCTCTGCA |
| | | | | (SEQ ID NO: 97) |
| Anti- | DIVLTQSPASLAVSL | QIQLVQSGPELEKP | GACATTGTGCTGACCCAATCTCCAGCTTCTTTGG | CAGATCCAGTTGGTGCAGTCTGGACCTGAGCTGG |
| body | GQRAIISCRASESV | GETVKISCFASGYT | CTGTGTCTCTAGGGCAGAGGGCCATCATATCCTG | AGAAGCCTGGAGAGACAGTCAAGATCTCCTGTAA |
| RH1 | DIYGNSFMHWCQQ | FTDYSMHWVKQAP | CAGAGCCAGTGAAAGTGTTGATATTTATGGCAATA | GGCTTCTGGTTATACCTTCACAGACTATTCAATGC |
| (A) | KPGQPRKLLIFRAS | GKGLKWMGWINTE | GTTTATGCACTGGTGCCAGCAGAAGCCAGGACA | ACTGGGTGAAGCAGGCTCCAGGAAAGGGTTTAAA |
| | NLESGIPARFSGSG | TGEPTYADDEKGRF | GCCACCCAAACTCCTCATCTTTCGTGCATCCAAC | GTGGATGGGCTGGATAAACACTGAGACTGGTGAG |
| | SRTDFTLTINPVEAD | AFSLETSASTAYLQI | CTAGAATCTGGGATCCCTGCCAGGTTCAGTGGCA | CCAACATATGCAGATGACTTCAAGGGACGGTTTG |
| | DVATYYCQQSYED | NNLKNEDTATYFCA | GTGGGTCTAGGACAGACTTCACCCTCACCATTAA | CCTTCTCGTTGGAAACCTCTGCCAGCACTGCCTA |
| | PWTFGGGTKLEIK | RSYAYAMDYWGQ | TCCTGTGGAGGCTGATGATGTTGCAACCTATTACT | TTTGCAGATCAACAACCTCAAAAATGAGGACACG |
| | (SEQ ID | GTSVTVSS | GTCAACAAAGTTATGAGGATCCGTGGACGTTCGG | GCTACATATTTCTGTGCTAGATCTTACGCCTATGC |
| | NO: 104) | (SEQ ID | TGGAGGCACCAAGCTGGAAATCAAA | TATGGACTACTGGGGTCAAGGAACCTCAGTCACC |
| | | NO: 105) | (SEQ ID NO: 106) | GTCTCCTCA (SEQ ID NO: 107) |
| Anti- | DIVLTQSPASLAVSL | QIQLVQSGPELKKP | GACATTGTGCTGACCCAATCTCCAGCTTCTTTGG | CAGATCCAGTTGGTGCAGTCTGGACCTGAGCTGA |
| body | GQRAIISCRASESV | GETVKISCKASGYT | CTGTGTCTCTAGGGCAGAGGGCCATCATATCCTG | AGAAGCCTGGAGAGACAGTCAAGATCTCCTGCAA |
| RH1 | DIYGNSFMHWCQQ | FTNYGMNWVKQAP | CAGAGCCAGTGAAAGTGTTGATATTTATGGCAATA | GGCTTCTGGGTATACCTTCACAAACTATGGAATGA |
| (B) | KPGQPPKLLIFRAS | GKGLKWMGWINTF | GTTTTATGCACTGGTGCCAGCAGAAGCCAGGACA | ACTGGGTGAAGCAGGCTCCAGGAAAGGGTTTAAA |
| | NLESGIPARFSGSG | TGEPSYADDFKGR | GCCACCCAAACTCCTCATCTTTCGTGCATCCAAC | GTGGATGGGCTGGATAAACACCTTCACTGGAGAG |
| | SRTDFTLTINPVEAD | FAFSLETSASTAYL | CTAGAATCTGGGATCCCTGCCAGGTTCAGTGGCA | CCATCATATGCTGATGACTTCAAGGGACGGTTTG |
| | DVATYYCQQSYED | QINNLKNEDTATYF | GTGGGTCTAGGACAGACTTCACCCTCACCATTAA | CCTTCTCTTTGGAAACCTCTGCCAGCACTGCCTAT |
| | PWTFGGGTKLEIK | CARNYGYPWFAYW | TCCTGTGGAGGCTGATGATGTTGCAACCTATTACT | TTGCAGATCAACAACCTCAAAAATGAGGACACGG |
| | (SEQ ID | GQGTLVTVSA | GTCAACAAAGTTATGAGGATCCGTGGACGTTCGG | CTACATATTTCTGTGCAAGAAACTACGGCTACCCC |
| | NO: 114) | (SEQ ID | TGGAGGCACCAAGCTGGAAATCAAA | TGGTTTGCTTACTGGGGCCAAGGGACTCTGGTCA |
| | | NO: 115) | (SEQ ID NO: 116) | CTGTCTCTGCA (SEQ ID NO: 117) |
| Anti- | DIVLTQSPASLAVSL | QIQLVQSGPELEKP | GACATTGTGCTGACACAGTCTCCTGCTTCCTTAG | CAGATCCAGTTGGTGCAGTCTGGACCTGAGCTGG |
| body | GQRATISYRASKSV | GETVKISCKASGYT | CTGTATCTCTGGGGCAGAGGGCCACCATCTCATA | AGAAGCCTGGAGAGACAGTCAAGATCTCCTGTAA |
| RH1 | STSGYSYMHWNQQ | FTDYSMHWVKQAP | CAGGGCCAGCAAAAGTGTCAGTACATCTGGCTAT | GGCTTCTGGTTATACCTTCACAGACTATTCAATGC |
| (C) | KPGQPPRLLIYLVS | GKGLKWMGWINTE | AGTTATATGCACTGGAACCAACAGAAACCAGGAC | ACTGGGTGAAGCAGGCTCCAGGAAAGGGTTTAAA |
| | NLESGIPARFSGSG | TGEPTYADDFKGRF | AGCCACCCAGACTCCTCATCTATCTTGTATCCAAC | GTGGATGGGCTGGATAAACACTGAGACTGGTGAG |
| | SRTDFTLTINPVEAD | AFSLETSASTAYLQI | CTAGAATCTGGGATCCCTGCCAGGTTCAGTGGCA | CCAACATATGCAGATGACTTCAAGGGACGGTTTG |
| | DVATYYCQQSYED | NNLKNEDTATYFCA | GTGGGTCTAGGACAGACTTCACCCTCACCATTAA | CCTTCTCGTTGGAAACCTCTGCCAGCACTGCCTA |
| | PWTFGGGTKLEIK | RSYAYAMDYWGQ | TCCTGTGGAGGCTGATGATGTTGCAACCTATTACT | TTTGCAGATCAACAACCTCAAAAATGAGGACACG |
| | (SEQ ID | GTSVTVSS | GTCAACAAAGTTATGAGGATCCGTGGACGTTCGG | GCTACATATTTCTGTGCTAGATCTTACGCCTATGC |
| | NO: 124) | (SEQ ID | TGGAGGCACCAAGCTGGAAATCAAA | TATGGACTACTGGGGTCAAGGAACCTCAGTCACC |
| | | NO: 125) | (SEQ ID NO: 126) | GTCTCCTCA (SEQ ID NO: 127) |
| Anti- | DIVLTQSPASLAVSL | QIQLVQSGPELKKP | GACATTGTGCTGACACAGTCTCCTGCTTCCTTAG | CAGATCCAGTTGGTGCAGTCTGGACCTGAGCTGA |
| body | GQRATISYRASKSV | GETVKISCKASGYT | CTGTATCTCTGGGGCAGAGGGCCACCATCTCATA | AGAAGCCTGGAGAGACAGTCAAGATCTCCTGCAA |
| RH1 | STSGYSYMHWNQQ | FTNYGMNWVKQAP | CAGGGCCAGCAAAAGTGTCAGTACATCTGGCTAT | GGCTTCTGGGTATACCTTCACAAACTATGGAATGA |
| (D) | KPGQPPRLLIYLVS | GKGLKWMGWINTF | AGTTATATGCACTGGAACCAACAGAAACCAGGAC | ACTGGGTGAAGCAGGCTCCAGGAAAGGGTTTAAA |
| | NLESGIPARFSGSG | TGEPSYADDFKGR | AGCCACCCAGACTCCTCATCTATCTTGTATCCAAC | GTGGATGGGCTGGATAAACACCTTCACTGGAGAG |
| | SRTDFTLTINPVEAD | FAFSLETSASTAYL | CTAGAATCTGGGATCCCTGCCAGGTTCAGTGGCA | CCATCATATGCTGATGACTTCAAGGGACGGTTTG |
| | DVATYYCQQSYED | QINNLKNEDTATYF | GTGGGTCTAGGACAGACTTCACCCTCACCATTAA | CCTTCTCTTTGGAAACCTCTGCCAGCACTGCCTAT |
| | PWTFGGGTKLEIK | CARNYGYPWFAYW | TCCTGTGGAGGCTGATGATGTTGCAACCTATTACT | TTGCAGATCAACAACCTCAAAAATGAGGACACGG |
| | (SEQ ID | GQGTLVTVSA | GTCAACAAAGTTATGAGGATCCGTGGACGTTCGG | CTACATATTTCTGTGCAAGAAACTACGGCTACCCC |
| | NO: 134) | (SEQ ID | TGGAGGCACCAAGCTGGAAATCAAA | TGGTTTGCTTACTGGGGCCAAGGGACTCTGGTCA |
| | | NO: 135) | (SEQ ID NO: 136) | CTGTCTCTGCA (SEQ D NO: 137) |
| Anti- | DVVMTQTPLSLPVS | QVQLQQPGAELVK | GATGTTGTGATGACCCAAACTCCACTCTCCCTGC | CAGGTCCAACTGCAGCAGCCTGGGGCTGAGCTG |
| body | LGDQASMSCRSSQ | PGASVKMSCKASG | CTGTCAGTCTTGGAGATCAAGCCTCCATGTCTTG | GTGAAGCCTGGGGCTTCAGTGAAGATGTCCTGCA |
| RF1 | SLVHSNGDSYLHW | YTFTNYWLHWVMQ | CAGATCTAGTCAGAGCCTTGTACACAGTAATGGA | AGGCTTCTGGCTACACCTTCACCAACTACTGGTT |
| | YLQKPGQSPKLLIY | RPGQGLEWIGMIDP | GACTCCTATTTACATTGGTACCTGCAGAAGCCAG | GCACTGGGTGATGCAGAGGCCTGGACAAGGCCT |
| | KVSNRLSGVPDRFS | SDSFTTYNQKFKGK | GCCAGTCTCCAAAGCTCCTGATCTACAAAGTTTCC | TGAGTGGATCGGAATGATTGATCCTTCTGATAGTT |
| | GSGSGTDFTLKISR | ATLTVDTSSSTAFM | AACCGATTGTCTGGGGTCCCAGACAGGTTCAGTG | TTACTACCTACAATCAAAAGTTCAAGGGCAAGGC |
| | VEAEDLGIYFCSQS | QLSSLTSEDSAVYY | GCAGTGGATCAGGGACAGATTTCACACTCAAGAT | CACATTGACTGTAGACACATCCTCCAGCACAGCC |
| | THVPYTFGGGTKLE | CTTVDHWGRGTTL | CAGCAGAGTGGAGGCTGAGGATCTGGGAATTTAT | TTCATGCAGCTCAGCAGCCTGACATCTGAAGACT |
| | IK | TVSS | TTCTGCTCTCAAAGTACACATGTTCCGTACACGTT | CTGCGGTCTATTATTGTACTACGGTGGACCACTG |
| | (SEQ ID | (SEQ ID | CGGAGGGGGGACCAAGCTGGAAATAAAA | GGGCCGAGGCACCACTCTCACAGTCTCCTCA |
| | NO: 144) | NO: 145) | (SEQ ID NO: 146) | (SEQ ID NO: 147) |
| Anti- | DVVMTOTPLSLPVS | QVQLQQPGAELVR | GATGTTGTGATGACCCAAACTCCACTCTCCCTGC | CAGGTCCAACTGCAGCAGCCTGGGGCTGAGCTG |
| body | LGDQASISCRSSQS | PGASVKLSCKASGY | CTGTCAGTCTTGGAGATCAAGCCTCCATCTCTTG | GTGAGGCCTGGGGCTTCAGTGAAGCTGTCTTGCA |
| RF3 | LVHSNGNTYLHWY | TFTNYWMNWWKQ | CAGATCTAGTCAGAGCCTTGTACACAGTAATGGA | AGGCTTCTGGCTACACCTTCACCAATTACTGGAT |
| | LQKPGQSPKLLIYIV | RPGQGLEWIGMIDP | AACACCTATTTACACTGGTACCTGCAGAAGCCAG | GAACTGGTGGAAACAGAGGCCTGGACAAGGCCT |
| | SNRFSGVPDRFGG | SDSETHYNQVFKD | GCCAGTCTCCAAAGCTCCTGATCTACATAGTTTCC | TGAATGGATTGGTATGATTGATCCTTCAGACAGTG |
| | SGSGTDFTLKISRV | KATLTVDISSTTAYM | AACCGATTTTTCTGGGGTCCCAGACAGGTTCGGTG | AAACTCACTACAATCAAGTGTTCAAGGACAAGGC |
| | EAEDLGVYFCSQST | HLSSLTSEDSAVYY | GCAGTGGATCAGGGACAGATTTCACACTCAAGAT | CACATTGACTGTAGACATATCCTCCACCACAGCCT |
| | HIPYTFGGGTKLEM | CARDFGWGQGTLV | CAGCAGAGTGGAGGCTGAGGATCTGGGAGTTTAT | ACATGCACCTCAGCAGCCTGACATCTGAGGACTC |
| | K | TVSA | TTCTGCTCTCAAAGTACACATATTCCGTACACGTT | TGCGGTCTATTACTGTGCAAGAGACTTCGGCTGG |
| | (SEQ ID | (SEQ ID | CGGAGGGGGGACCAAGCTGGAAATGAAA | GGCCAAGGGACTCTGGTCACTGTCTCTGCA |
| | NO: 154) | NO: 155) | (SEQ ID NO: 156) | (SEQ UD NO: 157) |
| Anti- | DVLMTQTPLSLPVS | QVQLQQSGAELVR | GATGTTTTGATGACCCAAACTCCACTCTCCCTGCC | CAGGTCCAGCTGCAGCAGTCTGGAGCTGAGCTG |
| body | LGDQASISCRSSQS | PGTSVKVSCKASG | TGTCAGTCTTGGAGATCAAGCCTCCATCTCTTGCA | GTAAGGCCTGGGACTTCAGTGAAGGTGTCCTGCA |
| R45 | IVHSNGNTYLEWYL | YAFTNYLIEWVKQR | GATCTAGTCAGAGTATTGTACATAGTAATGGAAAC | AGGCTTCTGGATACGCCTTCACTAATTCTTGATA |
| (A) | QKPGQSPKLLIYKV | PGQGLEWIGVINPG | ACCTATTTAGAATGGTACCTGCAGAAACCAGGCC | GAGTGGGTAAAGCAGAGGCCTGGACAGGGCCTT |
| | SNRFSGVPDRFSG | SGGTNYNEKFKGK | AGTCTCCAAAGCTCCTGATCTACAAAGTTTCCAAC | GAGTGGATTGGAGTGATTAATCCTGGAAGTGGTG |
| | SGSGTDFTLKISRV | ATLTADKSSSTAYM | CGATTTTCTGGGGTCCCAGACAGGTTCAGTGGCA | GTACTAACTACAATGAGAAGTTCAAGGGCAAGGC |
| | EAEDLGVYYCFQG | QLSSLTSDDSAVYF | GTGGATCAGGGACAGATTTCACACTCAAGATCAG | AACACTGACTGCAGACAAATCCTCCAGCACTGCC |
| | SHVPWTFGGGTKL | CARDDSAYWGQGT | CAGAGTGGAGGCTGAGGATCTGGGAGTTTATTAC | TACATGCAGCTCAGCAGCCTGACATCTGATGACT |
| | EIK | LVTVSA | TGTTTTCAAGGTTCACATGTTCCGTGGACGTTCG | CTGCGGTCTATTTCTGTGCAAGAGACGACAGTGC |
| | (SEQ ID | (SEQ ID | GTGGAGGCACCAAGCTGGAAATCAAA | TTACTGGGGCCAAGGGACTCTGGTCArTGTCTCT |
| | NO: 164) | NO: 165) | (SEQ ID NO: 166) | GCA (SEQ ID NO: 167) |
| Anti- | DIVLTQSPATLSVTP | QVQLQQSGAELVR | GATATTGTGCTAACTCAGTCTCCAGCCACCCTGT | CAGGTCCAGCTGCAGCAGTCTGGAGCTGAGCTG |
| body | GDSVSLSCRASQSI | PGTSVKVSCKASG | CTGTGACTCCAGGAGATAGCGTCAGTCTTTCCTG | GTAAGGCCTGGGACTTCAGTGAAGGTGTCCTGCA |
| RA5 | SNNLHWYQQKSHE | YAFTNYLIEWVKQR | CAGGGCCAGCCAAAGTATTAGCAACAACCTACAC | AGGCTTCTGGATACGCCTTCACTAATTACTTGATA |
| (B) | SPRLLIKYASQSISG | PGQGLEWIGVINPG | TGGTATCAACAAAAATCACATGAGTCTCCAAGGCT | GAGTGGGTAAAGCAGAGGCCTGGACAGGGCCTT |
| | IPSRFSGSGSGTDF | SGGTNYNEKFKGK | TCTCATCAAGTATGCTTCCCAGTCCATCTCTGGGA | GAGTGGATTGGAGTGATTAATCCTGGAAGTGGTG |
| | TLSINSVETEDFGM | ATLTADKSSSTAYM | TCCCCTCCAGGTTCAGTGGCAGTGGATCAGGGAC | GTACTAACTACAATGAGAAGTTCAAGGGCAAGGC |
| | YFCQQSNSWPLTF | QLSSLTSDDSAVYF | AGATTCACTCTCAGTATCAACAGTGTGGAGACTG | AACACTGACTGCAGACAAATCCTCCAGCACTGCC |
| | GAGTKLELK | CARDDSAYWGQGT | AAGATTTTGGAATGTATTTCTGTCAACAGAGTAAC | TACATGCAGCTCAGCAGCCTGACATCTGATGACT |
| | (SEQ ID | LTVSA | AGCTGGCCTCTCACGTTOGGTGCTGGGACCAAG | CTGCGGTCTATTTCTGTGCAAGAGACGACAGTGC |
| | NO: 174) | (SEQ ID | CTGGAGCTGAAA | TTACTGGGGCCAAGGGACTCTGGTCACTGTCTCT |
| | | NO: 175) | (SEQ ID NO: 176) | GCA (SEQ ID NO: 177) |
| Anti- | DVLMTQTPLSLPVS | QVQLQQPGAELVK | GATGTTTTGATGACCCAAACTCCACTCTCCCTGCC | CAGGTCCAACTGCAGCAGCCTGGGGCTGAGCTT |
| body | LGDQASISCRSSQS | PGASVKLSCKASGVY | TGTCAGTCTTGGAGATCAAGCCTCCATCTCTTGCA | GTGAAGCCTGGGGCTTCAGTGAAGCTGTCCTGCA |
| RD3 | IVHSNGNTYLEWYL | TFTSYWMHWVKQR | GATCTAGTCAGAGCATTGTACATAGTAATGGAAAC | AGGCTTCTGGCTACACCTTCACCAGCTACTGGAT |
| | QKPGQSPKLLIYKV | PGQGLEWIGEIDPS | ACCTATTTAGAATGGTACCTGCAGAAACCAGGCC | GCACTGGGTGAAGCAGAGGCCTGGACAAGGCCT |
| | SNRFSGVPDRFSG | DSYTNYNQKFKGK | AGTCTCCAAAGCTCCTGATCTACAAAGTTTCCAAC | TGAGTGGATCGGAGAGATTGATCCTTCTGATAGT |
| | SGSGTDFTLKISRV | ATLTVDKSSSTAYM | CGATTTTCTGGGGTCCCAGACAGGTTCAGTGGCA | TATACTAACTACAATCAAAAGTTCAAGGGCAAGGC |
| | EAEDLGVYYCFQG | QLSSLTSEDSAVYY | GTGGATCAGGGACAGATTTCACACTCAAGATCAG | CACATTGACTGTAGACAAATCCTCCAGCACAGCC |
| | SHVPWTFGGGTKL | CANMITTQGYFDY | CAGAGTGGAGGCTGAGGATCTGGGAGTTTATTAC | TACATGCAGCTCAGCAGCCTGACATCTGAGGACT |
| | EIK | WGQGTTLTVSS | TGCTTTCAAGGTTCACATGTTCCGTGGACGTTCG | CTGCGGTCTATTACTGTGCAAATATGATTACGACG |
| | (SEQ ID | (SEQ ID | GTGGAGGCACCAAGCTGGAAATCAAA | CAGGGCTACTTTGACTACTGGGGCCAAGGCACCA |
| | NO: 184) | NO: 185) | (SEQ ID NO: 186) | CTCTCACAGTCTCCTCA |
| | | | | (SEQ ID ND: 187) |
| Anti- | DVVMTQTPLSLPVS | EVQLQQSGAELVK | GATGTTGTGATGACCCAAACTCCACTCTCCCTGC | GAGGTTCAGCTGCAGCAGTCTGGGGCAGAGCTT |
| body | LGDQASISCRSSLS | PGASVKLSCTASGL | CTGTCAGTCTTGGAGATCAAGCCTCCATCTCTTGT | GTGAAGCCAGGGGCCTCAGTCAAGTTGTCCTGCA |
| RC1 | LVYSNGNTYLHWY | NIKDTYIHWVKQRP | AGATCTAGTCTGAGCCTTGTATACAGTAATGGAAA | CAGCTTCTGGCCTCAACATTAAAGACACCTATATA |
| | QQKPGQSPKLLIFK | EQGLEWIGRIDPAN | CACCTATTTACATTGGTACCAGCAGAAGCCAGGC | CACTGGGTGAAGCAGAGGCCTGAACAGGGCCTG |
| | VSNRFSGVPDRFS | GNTKYDPKFQDKA | CAGTCTCCAAAGCTCCTGATCTTCAAAGTTTCCAA | GAGTGGATTGGAAGGATTGATCCTGCGAATGGAA |
| | GSGSGTDFTLKISR | TITADTSSNTAYLHL | CCGATTTTCTGGGGTCCCAGACAGGTTCAGTGGC | ATACTAAATATGACCCGAAGTTCCAGGACGGC |
| | VEAEDLGVYFCSQ | SSLTSEDTAVYYCT | AGTGGATCAGGGACAGATTTCACACTCAAGATCA | CACTATAACAGCAGACACATCCTCCAACACAGCC |
| | NTHVPFTFGSGTKL | NGNWDWFAYWGQ | GCAGAGTGGAGGCTGAGGATCTGGGAGTTTATTT | TACCTGCACOTCAGCAGCCTGACATCTGAGGACA |
| | EIK | GTLVTVSA | CTGCTCTCAAAATACACATGTTCCATTCACGTTCG | CTGCCGTCTATTACTGTACTAATGGGAACTGGGA |
| | (SEQ ID | (SEQ ID | GCTCGGGGACAAAGTTGGAAATAAAA | CTGGTTTGCTTACTGGGGCCAAGGGACTCTGGTC |
| | NO: 194) | NO: 195) | (SEQ ID NO: 196) | ACTGTCTCTGCA |
| | | | | (SEQ ID NO: 197) |
| Anti- | DIVMTQTPLSLPVSL | EVQLQQSGAELVR | GATATTGTGATGACCCAAACTCCACTCTCCCTGC | GAGGTTCAGCTGCAGCAGTCTGGGGGTGAGCTT |
| body | GDQASISCKSGQSL | PGALVKLSSKASGF | CTGTCAGTCTTGGAGATCAAGCCTCCATCTCTTG | GTGAGGCCAGGGGCCTTAGTCAAGTTGTCCTCCA |
| RF2 | VHSNGNTYLRWYL | NIKDYYMHWVKQR | CAAATCTGGTCAGAGCCTTGTACACAGTAATGGA | AAGCTTCTGGCTTCAACATTAAAGACTACTATATG |
| | QKPGQSPKLLIYKV | PEQGLEWIGWIDPE | AACACCTATTTACGTTGGTACCTGCAGAAGCCAG | CACTGGGTGAAGCAGAGGCCTGAACAGGGCCTG |
| | SNRFSGVPDRFSG | NGNTIYDPKFQGKA | GCCAGTCTCCAAAGCTCCTGATCTACAAAGTTTCC | GAGTGGATTGGATGGATTGATCCTGAGAATGGTA |
| | SGSGTDFTLKISRV | TITADSSNTTYLQL | AACCGATTTTCTGGGGTCCCAGACAGGTTCAGTG | ATACTATATATGACCCGAAGTTCCAGGGCAAGGC |
| | EAEDLGVYFCSQST | SSLTSEDTAVYYCT | GCAGTGGATCAGGGACAGATTTCACACTCAAGAT | CACTATAACAGCAGACACATCCTCCAACACAACCT |
| | HVPLTFGAGTKLEL | REDYWGQGTSVTV | CAGCAGAGTGGAGGCTGAGGATCTGGGAGTTTAT | ACCTCCAGCTCAGCAGCCTGACATCTGAGGACAC |
| | K | SS | TTCTGCTCTCAAAGTACACATGTTCCGCTCACGTT | TGCCGTCTATTACTGTACTCGAGAGGACTACTGG |
| | (SEQ ID | (SEQ ID | CGGTGCTGGGACCAAGCTGGAGCTGAAA | GGTCAAGGAACCTCAGTCACCGTCTCCTCA |
| | NO: 204) | NO: 205) | (SEQ ID NO: 206) | (SEQ ID NO: 207) |

TABLE 3
Framework region sequences (light chain and heavy chain)

| Anti- | | | | | | | | |
|---|---|---|---|---|---|---|---|---|
| body | | | | | | | | |
| ID- | LFR1 | LFR2 | LFR3 | LFR4 | HFR1 | HFR2 | HFR3 | HFR4 |
| Anti- | QIVILTQS | WYQQKSGTSPK | GVPARFSGSGS | FGGGTKLEIK | QVQLKQSGPGL | WVRQSPGKGLE | RLSITKDNSKSQ | WGQGTLV |
| body | PAIMSASP | RWIY | GTSYSLTISSME | (SEQ ID | VQPSQSLSITCT | WLG | VFFKMNSLQAD | TVSA |
| C3 | GEKVTMTC | (SEQ ID | AEDAATYYC | NO: 25) | VSGFSLT | (SEQ ID | DTAIYYCAK | (SEQ ID |
| | (SEQ ID | NO: 23) | (SEQ ID | | (SEQ ID | NO: 27) | (SEQ ID | NO: 29) |
| | NO: 22) | | NO: 24) | | NO: 26) | | NO: 28) | |
| Anti- | DIVMSQSP | WYQQKPGDSPK | GVPDRFTGSGS | FGSGTKLEIK | EVQLQQSGAEL | WVKQRPEQGLE | KATITADTSSNT | WGQGTLV |
| body | SSLAVSVG | LLIY | GTDFTLTISSVK | (SEQ ID | VKPGASVKLSCT | WIG | AYLQLSSLTSED | TVSA |
| D3 | EKVTMSC | (SEQ ID | AEDLAVYYC | NO: 33) | ASGFNIK | (SEQ ID | TAVYYCAK | (SEQ ID |
| | (SEQ ID | NO: 31) | (SEQ ID | | (SEQ ID | NO: 35) | (SEQ ID | NO: 37) |
| | NO: 30) | | NO: 32) | | NO: 34) | | NO: 36) | |
| Anti- | DIVLTQSP | WCQQKPGQPP | GIPARFSGSGSR | FGGGTKLEIK | QIQLVQSGPELK | WVKQAPGKGLK | RFAESLETSAST | WGQGTLV |
| body | ASLAVSLG | KLLIF | TDFTLTINPVEA | (SEQ ID | KPGETVKISCKA | WMG | AYLQINNLKDED | TVSA |
| RD1 | QRAIISC | (SEQ ID | DDVATYYC | NO: 211) | SGYTFK | (SEQ ID | TATYFCAR | (SEQ ID |
| (A) | (SEQ ID | NO: 209) | (SEQ ID | | (SEQ ID | NO: 213) | (SEQ ID | NO: 215) |
| | NO: 208) | | NO: 210) | | NO: 212) | | NO: 214) | |
| Arti- | DIVLTQSP | WCQQKPGQPP | GIPARFSGSGSR | FGGGTKLEIK | QIQLVQSGPELE | WVKQAPGKGLK | PFAFSLETSAST | WGQGTSV |
| body | ASLAVSLG | KLLIF | TDFTLTINPVEA | (SEQ ID | KPGETVKISCKA | WMG | AYLQINNLKNED | TVSS |
| RD1 | QRAIISC | (SEQ ID | DDVATYYC | NO: 219) | SGYTFT | (SEQ ID | TATYFCAR | (SEQ ID |
| (B) | (SEQ ID | NO: 217) | (SEQ ID | | (SEQ ID | NO: 221) | (SEQ ID | NO: 223) |
| | NO: 216) | | NO: 218) | | NO: 220) | | NO: 222) | |
| Anti- | DIVMTQSQ | WYQQKPGQSPK | GVPDRFTGSGS | FGAGTKLELK | QIQLVQSGPELK | WVKQAPGKGLK | RFAFSLETSAST | WGQGTLV |
| body | KFMSTSVG | ALIY | GTDFTLTISNVQ | (SEQ ID | KPGETVKISCKA | WMG | AYLQINNLKDED | TVSA |
| RD1 | DRVSVTC | (SEQ ID | SEDLAEYFC | NO: 227) | SGYTFK | (SEQ ID | TATYFCAR | (SEQ ID |
| (C) | (SEQ ID | NO: 225) | (SEQ ID | | (SEQ ID | NO: 229) | (SEQ ID | NO: 231) |
| | NO: 224) | | NO: 226) | | NO: 228) | | NO: 230) | |
| Anti- | DIVMTQSQ | WYQQKPGQSPK | GVPDRFTGSGS | FGAGTKLELK | QIQLVQSGPELE | WVKQAPGKGLK | RFAFSLETSAST | WGQGTSV |
| body | KFMSTSVG | ALIY | GTDFTLTISNVQ | (SEQ ID | KPGETVKISCKA | WMG | AYLQINNLKNED | TVSS |
| RD1 | DRVSVTC | (SEQ ID | SEDLAEYFC | NO: 235) | SGYTFT | (SEQ ID | TATYFCAR | (SEQ ID |
| (D) | (SEQ ID | NO: 233) | (SEQ ID | | (SEQ ID | NO: 237) | (SEQ ID | NO: 239) |
| | NO: 232) | | NO: 234) | | NO: 236) | | NO: 238) | |
| Anti- | DVVMTQTP | WYLQKPGQSPK | GVPDRFSGSGS | FGSGTKLEIK | EVQLQQSGAEL | WVKQRPEQGLE | KATVTADTSSNT | WGQGTLV |
| body | LSLPVSLG | LLIY | GTDFTLKISRVE | (SEQ ID | VKPGASVTLSCT | WIG | AYLQLSSLTSED | TVSA |
| RE1 | DQASISC | (SEQ ID | AEDLGVYFQ | NO: 243) | ASGFNIK | (SEQ ID | TAVYFCSN | (SEQ ID |
| | (SEQ ID | NO: 241) | (SEQ ID | | (SEQ ID | NO: 255) | (SEQ ID | NO: 247) |
| | NO: 240) | | NO: 242) | | NO: 244) | | NO: 246) | |
| Anti- | DIVMTQSQ | WYQQKPGQSPK | GVPDRFTGSGS | FGGGTKLELK | QVQLKESGPGL | WVRQPPGKGLE | RLRITKDNFKSQ | WGQGTLV |
| body | EFLSTLVG | TLIY | GTDFTLTISSVQ | (SEQ ID | VAPSQSLTITCT | WLG | VFLHLNSLQTDD | TVSA |
| RB6 | DRVSVTC | (SEQ ID | SEDLAKYFC | NO: 251) | VSGFSLT | (SEQ ID | TATYYCAR | (SEQ ID |
| | (SEQ ID | NO: 249) | (SEQ ID | | (SEQ ID | NO: 253) | (SEQ ID | NO: 255) |
| | NO: 248) | | NO: 250) | | NO: 252) | | NO: 254) | |
| Anti- | DIVLTQSP | WCQQKPGQPP | GIPARFSGSGSR | FGGGTKLEIK | QIQLVQSGPELE | WVKQAPGKGLK | RFAFSLETSAST | WGQGTSV |
| body | ASLAVSLG | KLLIF | TDFTLTINPVEA | (SEQ ID | KPGETVKISCKA | WMG | AYLQINNLKNED | TVSS |
| RH1 | QRAIISC | (SEQ ID | DDVATYYC | NO: 259) | SGYTFT | (SEQ ID | TATYFCAR | (SEQ ID |
| (A) | (SEQ ID | NO: 257) | (SEQ ID | | (SEQ ID | NO: 261) | (SEQ ID | NO: 263) |
| | NO: 256) | | NO: 258} | | NO: 260) | | NO: 262) | |
| Anti- | DIVLTQSP | WCQQKPGQPP | GIPARFSGSGSR | FGGGTKLEIK | QIQLVQSGPELK | WVKQAPGKGLK | RFAFSLETSAST | WGQGTLV |
| body | ASLAVSLG | KLLIF | TDFTLTINPVEA | (SEQ ID | KPGETVKISCKA | WMG | AYLQINNLKNED | TVSA |
| RH1 | QRAIISC | (SEQ ID | DDVATYYC | NO: 267) | SGYTFT | (SEQ ID | TATYFCAR | (SEQ ID |
| (B) | (SEQ ID | NO: 265) | (SEQ ID | | (SEQ ID | NO: 269) | (SEQ ID | NO: 271) |
| | NO: 264) | | NO: 266) | | NO: 268) | | NO: 270) | |
| Anti- | DIVLTQSP | WNQQKPGQPP | GIPARFSGSGSR | FGGGTKLEIK | QIQLVQSGPELE | WVKQAPGKGLK | RFAFSLETSAST | WGQGTSV |
| body | ASLAVSLG | RLLIY | TDFTLTINPVEA | (SEQ ID | KPGETVKISCKA | WMG | AYLQINNLKNED | TVSS |
| RH1 | QRATISY | (SEQ ID | DDVATYYC | NO: 275) | SGYTFT | (SEQ ID | TATYFCAR | (SEQ ID |
| (C) | (SEQ ID | NO: 273) | (SEQ ID | | (SEQ ID | NO: 277) | (SEQ ID | NO: 279) |
| | NO: 272) | | NO: 274) | | NO: 276) | | NO: 278) | |
| Anti- | DIVLTQSP | WNQQKPGQPP | GIPARFSGSGSR | FGGGTKLEIK | QIQLVQSGPELK | WVKQAPGKGLK | RFAFSLETSAST | WGQGTLV |
| body | ASLAVSLG | RLLIY | TDFTLTINPVEA | (SEQ ID | KPGETVKISCKA | WMG | AYLQINNLKNED | TVSA |
| (D) | QRATISY | (SEQ ID | DDVATYYC | NO: 283) | SGYTFT | (SEQ ID | TATYFCAR | (SEQ ID |
| | (SEQ ID | NO: 281) | (SEQ ID | | (SEQ ID | (NO: 285) | (SEQ ID | NO: 287) |
| | NO: 280) | | NO: 282) | | NO: 284) | | NO: 286) | |
| Anti- | DVVMTQTP | WYLQKPGQSPK | GVPDRFSGSGS | FGGGTKLEIK | QVQLQQPGAEL | WVMQRPGQGL | KATLTVDTSSST | WGRGTTL |
| body | LSLPVSLG | LLIY | GTDFTLKISRVE | (SEQ ID | VKPGASVKMSC | EWIG | AFMQLSSLTSED | TVSS |
| RF1 | DQASMSC | (SEQ ID | AEDLGIYFC | NO: 291) | KASGYTFT | (SEQ ID | SAVYYCTT | (SEQ ID |
| | (SEQ ID | NO: 289) | (SEQ ID | | (SEQ ID | NO: 293) | (SEQ ID | NO: 295) |
| | NO: 288) | | NO: 290) | | NO: 292) | | NO: 294) | |
| Anti- | DVVMTQTP | WYLQKPGQSPK | GVPDRFGGSGS | FGGGTKLEMK | QVQLQQPGAEL | WWKQRPGQGL | KATLTVDISSTTA | WGQGTLV |
| body | LSLPVSLG | LLIY | GTDFTLKISRVE | (SEQ ID | VRPGASVKLSC | EWIG | YMHLSSLTSEDS | TVSA |
| RF3 | DQASISC | (SEQ ID | AEDLGVYFC | NO: 299) | KASGYTFT | (SEQ ID | AVYYCAR | (SEQ ID) |
| | (SEQ ID | NO: 297) | (SEQ ID | | (SEQ ID | NO: 301) | (SEQ ID | NO: 303) |
| | NO: 296) | | NO: 298) | | NO: 300) | | NO: 302) | |
| Anti- | DVLMTQTP | WYLQKPGQSPK | GVPDRFSGSGS | FGGGTKLEIK | QVQLQQSGAEL | WVKQRPGQGLE | KATLTADKSSST | WGQGTLV |
| body | LSLPVSLG | LLIY | GTDFTLKISRVE | (SEQ ID | VRPGTSVKVSC | WIG | AYMQLSSLTSD | TVSA |
| RA5 | DQASISC | (SEQ ID | AEDLGVYYC | NO: 307) | KASGYAFT | (SEQ ID | DSAVYFCAR | (SEQ ID |
| (A) | (SEQ ID | NO: 305) | (SEQ ID | | (SEQ ID | NO: 309) | (SEQ ID | NO: 311) |
| | NO: 304) | | NO: 306) | | NO: 308) | | NO: 310) | |
| Anti- | DIVLTQSP | WYQQKSHESPR | GIPSRFSGSGSG | FGAGTKLELK | QVQLQQSGAEL | WVKQRPGQGLE | KATLTADKSSST | WGQGTLV |
| body | VATLSTPG | LLIK | TDFTLSINSVETE | (SEQ ID | VRPGTSVKVSC | WIG | AYMQLSSLTSD | TVSA |
| RA5 | DSVSLSC | (SEQ ID | DFGMYFC | NO: 315) | KASGYAFT | (SEQ ID | DSAVYFCAR | (SEQ ID |
| (B) | (SEQ ID | NO: 313) | (SEQ ID | | (SEQ ID | NO: 317) | (SEQ ID | NO: 319) |
| | NO: 312) | | NO: 314) | | NO: 316) | | NO: 318) | |
| Anti- | DVLMTQTP | WYLQKPGQSPK | GVPDRFSGSGS | FGGGTKLEIK | QVQLQQPGAEL | WVKQRPGQGLE | KATLTVDKSSST | WGQGTTL |
| body | LSLPVSLG | LLIY | GTDFTLKISRVE | (SEQ ID | VKPGASVKLSCK | WIG | AYMQLSSLTSED | TVSS |
| RD3 | DQASISC | (SEQ ID | AEDLGVYYC | NO: 323) | ASGYTFT | (SEQ ID | SAVYYCAN | (SEQ ID |
| | (SEQ ID | NO: 321) | (SEQ ID | | (SEQ ID | NO: 325) | (SEQ ID | NO: 327) |
| | NO: 320) | | NO: 322) | | NO: 324) | | NO: 326) | |
| Anti- | DVVMTQTP | WYQQKPGQSPK | GVPDRFSGSGS | FGSGTKLEIK | EVQLQQSGAEL | WVKQRPEQGLE | KATITADTSSNT | WGQGTLV |
| body | LSLPVSLG | LLIF | GTDFTLKISRVE | (SEQ ID | VKPGASVKLSCT | WIG | AYLHLSSLTSED | TVSA |
| RC1 | DQASISC | SEQ ID | AEDLGVYFC | NO: 331) | ASGLNIK | (SEQ ID | TAVYYCTN | (SEQ ID |
| | (SEQ ID | NO: 329) | (SEQ ID | | (SEQ ID | NO: 333) | (SEQ ID | NO: 335) |
| | NO: 328) | | NO: 330) | | NO: 332) | | NO: 334) | |
| Anti- | DIVMTQTP | WYLQKPGQSPK | GVPDRFSGSGS | FGAGTKLELK | EVQLQQSGAEL | WVKQRPEQGLE | KATITADTSSNT | WGQGTSV |
| body | LSLPVSLG | LLIY | GTDFTLKISRVE | (SEQ ID | VRPGALVKLSSK | WIG | TYLQLSSLTSED | TVSS |
| RF2 | DQASISC | (SEQ ID | AEDLGVYFC | NO: 339) | ASGFNIK | (SEQ ID | TAVYYCTR | (SEQ ID |
| | (SEQ ID | NO: 337) | (SEQ ID | | (SEQ ID | NO: 341) | (SEQ ID | NO: 343) |
| | NO: 336) | | NO: 338) | | NO: 340) | | NO: 342) | |

The present invention includes the following non-limiting Examples.

### EXAMPLES

### Example 1

Production of IL-37 Inhibitory Antibodies

Immunisation and Serum Titre: Mice were immunised intraperitonealy 3 times at two-week intervals with a combination of 16 μg of IL-37 antigen and an immune adjuvant (Sigma-Aldrich cat# S6322) in combination with methylated CpG. A serum sample was collected from the immunized mice and reactivity to the antigen was tested by ELISA at a dilution of 1:250 and 1:1250 and compared to a pre-immunization sample. The mouse with the highest titre was selected for fusion.

The IL-37 antigen used was an untagged antigen of human origin, recombinant (rec) IL-37 (46-218):

(SEQ ID NO: 1)
GSVHTSPKVKNLNPKKFSIHDQDHKVLVLDSGNLIAVPDKNYIRPEIFFA
LASSLSSASAEKGSPILLGVSKGEFCLYCDKDKGQSHPSLQLKKEKLMKL
AAQKESARRPFIFYRAQVGSWNMLESAAHPGWFICTSCNCNEPVGVTDKF
ENRKHIEFSFQPVCKAEMSPSEVSD

Hybridoma Fusion: To generate hybridoma cells the mouse spleen was excised, dissociated into a single cell suspension and fused to SP2/0-Ag14 myeloma cells using polyethylene glycol. The resultant hybridoma cells were grown in Azaserine Hypoxantine containing medium in 20×96 well tissue culture plates.

### Example 2

Screening Antibodies for Binding to IL-37

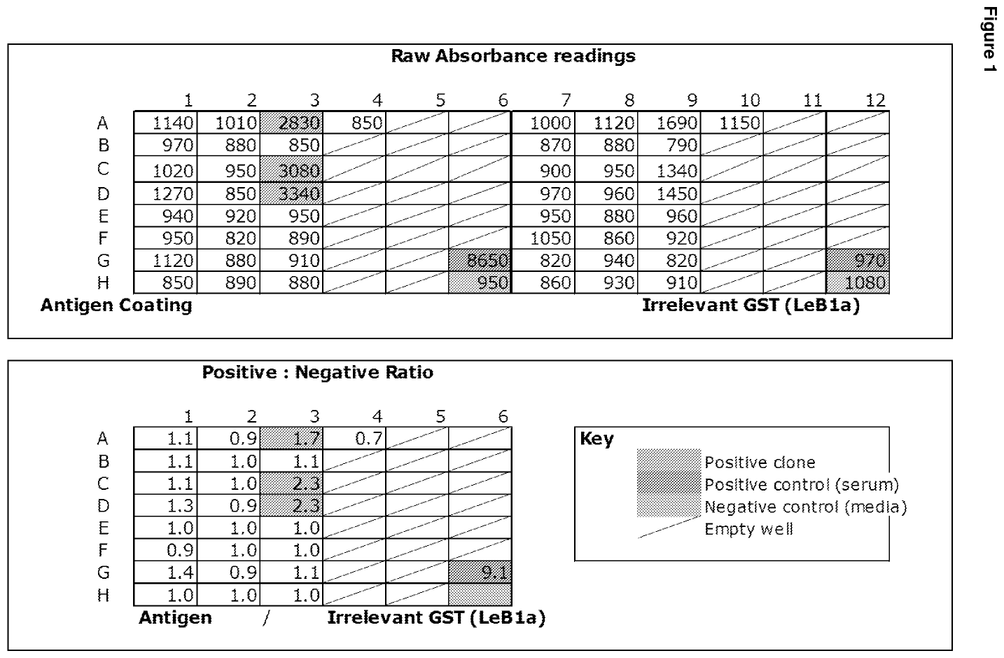

Hybridoma colonies were grown for 10 days at which point the number of hybridoma colonies was determined and after a further 3 days incubation an aliquot of antibody supernatant taken for screening. The supernatant was assayed for reactivity to the antigen and any screening samples, firstly by microarray followed by ELISA of any IgG microarray positive clones (FIG. 1). The positive clones include C3, D3 and A3 (also referred to herein as RC3, RD3 and RA3, respectively).

A serum sample was collected from the immunized mice and reactivity to the antigen was tested by ELISA at a dilution of 1:250 and 1:1250 and compared to a pre-immunization sample. The mouse with the highest titre was selected for fusion.

The results shown in FIG. 1 are the absorbance readings (Raw absorbance readings) as well and the Positive:Negative ratios.

Before sequencing, hybridoma supernatants were screened for IL-37-blocking activity; based on the results from this functional assay (which showed good blocking activity for clones C3 and D3), clones C3 and D3 were selected for sequencing.

Clones C3 and D3 were then sequenced and the results of that sequencing are shown in Tables 1 to 3 with CDRs determined using IMGT analysis.

### Example 3

Binding Specificity of Antibodies

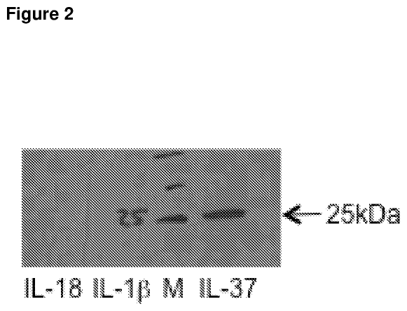

An immunoblot of rec IL-18, rec IL1β and rec IL-37 for detection of specificity and cross reactivity of the antibodies generated was performed. The blot was probed with anti-IL-37 hybridoma supernatant. No cross reactivity with rec IL-18 or rec IL-1β was observed highlighting the specificity of the generated antibodies for IL-37 (FIG. 2). While the antibody (clone D3) detects IL-37, IL-18 (a closely related IL-1 family member that IL-37 shares critical amino acids and a receptor chain with) was not detected.

### Example 4

Blocking Activity of Endogenous IL-37 by Antibodies

Freshly isolated human PBMC were resuspended at 2.5×10⁵ cells/ml in RPMI supplemented with 1% human serum and 1:500 MycoZap Plus-PR and treated with vehicle (Veh) or LPS (500 pg/ml) at time point to. At to or 1, 3 or 6 h thereafter, IL-37 antibody (clone D3) was added as indicated. Twenty h after to, supernatants were analyzed for IL-1β by ELISA. Depicted is the mean IL-1β concentration±SEM; n=3 donors. **, P<0.01; ***, P<0.001 compared to LPS alone. ###, P<0.001 compared to LPS+37bl1 one h after t₀.

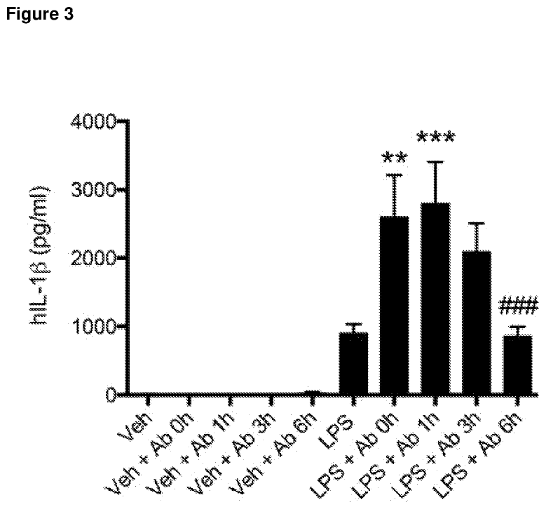

The results in FIG. 3 show that simultaneous treatment with LPS and IL-37 antibody leads to a significant, nearly 3-fold increase in IL-1β at 20 h compared to cells receiving LPS only. This increase in IL-1β is due to IL-37 antibody blocking the effects of endogenous IL-37 released from the cells at any time during the duration of the experiment. The same increase in IL-1β is observed when the antibody is added 1 h after LPS stimulation, indicating that there is no increase in extracellular IL-37 function in the first hour after stimulation of PBMC with LPS.

However, if the antibody is added 3 h after LPS, the IL-1 concentration drops, which is due to some IL-37 being released before addition of the IL-37 antibody which can then exert its anti-inflammatory effects (FIG. 3). If the antibody is added 6 h after LPS-stimulation, the IL-1β concentration equals that observed in cells that received LPS only. This observation shows that IL-37 bioactivity released from the cells after the 6 h time point does not make a significant contribution to the anti-inflammatory effects of IL-37 in this experiment.

### Example 5

Blocking Activity of Recombinant IL-37 by Antibodies

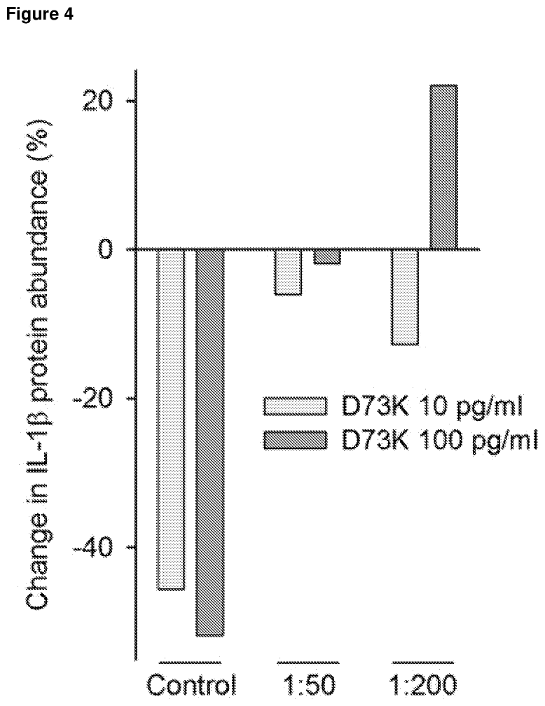

PBMC were isolated from peripheral venous blood of healthy volunteers by density gradient centrifugation as described (Nold et al., Biochemical Pharmacology 66, 505-510 (2003)). PBMC were plated in RPMI 1640 medium supplemented with L-glutamine, 25 mM HEPES (Thermo Fisher Scientific), 1% v/v human serum (Sigma Aldrich) and 1:500 MycoZap Plus-PR (Lonza), then pre-treated for 30 min with either vehicle or rec IL-37 as indicated, before stimulation with 50 pg/ml LPS (O55:B5, Sigma Aldrich) for 20 h. The IL-37-blocking antibody (clone D3) was generated by Monash Antibody Technologies Facility and the hybridoma supernatant was characterized in regards to IL-37-blocking capacity using a bioactivity assay (FIG. 4). Freshly isolated PBMC were first treated with the indicated dilutions of the IL-37-blocking antibody or control, followed by addition of the indicated concentrations of D73K⁴⁶⁻²¹⁸ or vehicle 30 min later, and 50 pg/ml LPS another 30 min later. IL-1β protein abundance was determined in the culture supernatants 20 h after addition of LPS, and percent changes afforded by treatment with the IL-37b variants in the presence or absence of the IL-37-blocking antibody were calculated and graphed±SEM; n=2 biological replicates from one exemplary donor.

### Example 6

Blocking of Transfected Natural or Monomeric IL-37 by Antibodies

Constructs expressing Natural¹⁻²¹⁸, D73K¹⁻²¹⁸ or Y85A¹⁻²¹⁸ were transfected into THP-1 cells. Following differentiation with PMA (50 ng/ml) for 24 h and a medium change, cultures pre-treated with hybridoma supernatant of LI-37 blocking antibody clone D3 (1:50 dilution) or control, then stimulated with 250 ng/ml LPS or vehicle. The fold-changes in IL-1β protein abundance between pairs of cultures treated with the IL-37-blocking antibody and vehicle-treated cultures (transfected with identical constructs, i.e. pairs of Control, Natural, D73K, or Y85A (D73K: IL-37 with a mutation from Asp to Lys at position 73; Y85A with a mutation from Tyr to Ala at position 85) and stimulated with LPS) were calculated and are graphed as mean±SEM; n=4 biological repeats for each LPS-stimulated condition; statistical significance was assessed using ANOVA on ranks and Student-Newman-Keuls post-hoc analysis; *, P<0.05 for Control vs IL-37b transfection; #, P<0.05 for Natural¹⁻²¹⁸ vs D73K¹⁻²¹⁸ or Y85A¹⁻²¹⁸. Dashed line indicates background of IL-1β induction by the IL-37-blocking antibody in Control—(i.e. not IL-37b) transfected cultures.

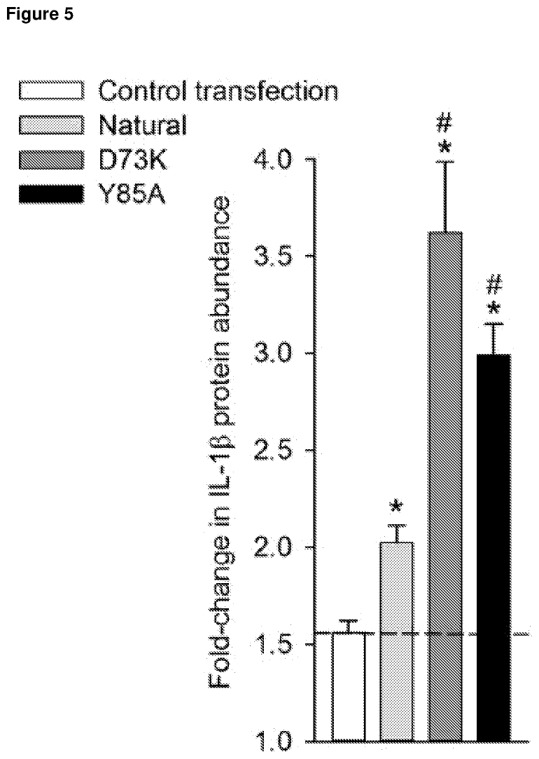

When an IL-37-blocking antibody (clone D3) was added to the transfected cultures before stimulation with LPS, we observed an increase in IL-1β upon transfection of each of the variants (FIG. 5). However, this increase was markedly more pronounced when the monomeric D73K¹⁻²¹⁸ and Y85A¹⁻²¹⁸ variants were transfected compared to dimeric Natural¹⁻²¹⁸ IL-37b.

### Example 7

Blocking Activity of Recombinant IL-37 by Antibodies: IL-1β Production

PBMC from healthy volunteers (n=2) were isolated and pre-treated with various dilutions of the hybridoma supernatants (clone C3 at 1:5 to 1:500) for 30 minutes, followed by treatment with Natural recombinant IL-37 (rIL-37) at various concentrations (from 1 pg/mL to 10 ng/mL) for 1 hour prior to LPS stimulation (500 pg/mL). After an overnight incubation, supernatants were collected and IL-1β was measured by ELISA.

RecIL-37 reduces IL-1β release from PBMC, however when the hybridoma supernatant from clone C3 is added, this anti-inflammatory activity is reduced or lost.

### Example 8

Blocking Activity of Recombinant IL-37 by Antibodies: IL-1β Production

Experiments involving human blood were approved by the Monash Health Human Research Ethics Committee. PBMCs were isolated from fresh, peripheral venous blood of healthy donors by density-gradient centrifugation using Leucosep Tubes (Greiner Bio-One, Germany), per the manufacturer's instructions. Isolated PBMCs were resuspended in RPMI 1640 (Thermo Fisher Scientific, USA) supplemented with 1% human serum (Sigma-Aldrich, USA) and 1:500 MycoZap Plus-PR (Lonza, Switzerland) and seeded in a 96-well plate. Cells were then rested for 1 h prior to addition of hybridoma clone supernatant (clone RD1). After 30 min incubation, recombinant IL-37 was added and incubated for a further 1 h before overnight stimulation with 200 pg/ml LPS (Sigma-Aldrich). IL-1β was measured in the supernatant by ELISA (BD Bioscience, USA) according to manufacturer's instructions.

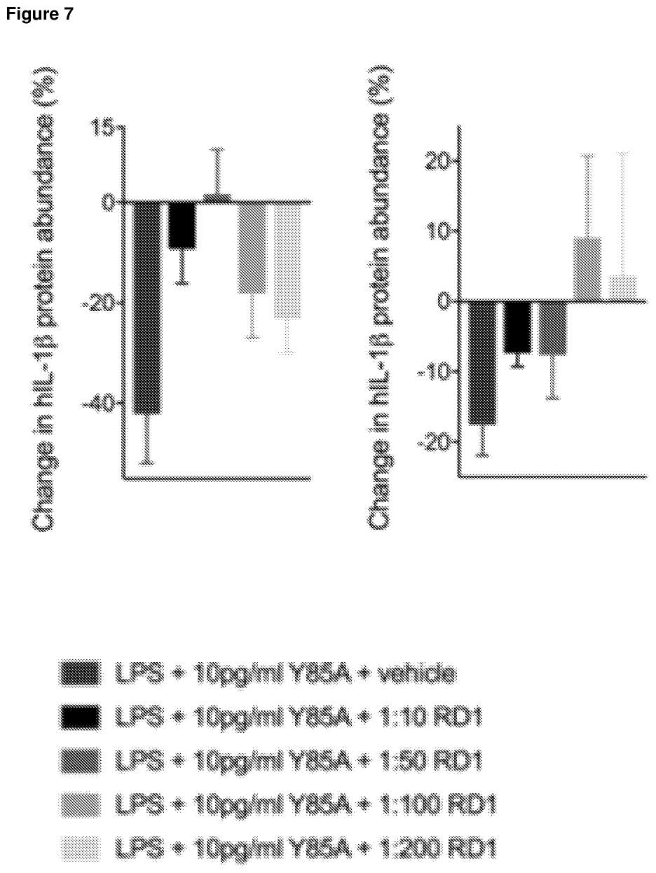

The increase in IL-1β abundance induced by LPS stimulation is reduced by up to 40% with the addition of recombinant IL-37. Pre-treatment with supernatant from clone RD1 prior to addition of IL-37 reversed the anti-inflammatory activity of IL-37, as determined by IL-1β abundance (FIG. 7).

### Example 9

Blocking Activity of Recombinant IL-37 by Antibodies: IL-6 Production

All animal work was approved by Monash University's Animal Ethics Committee and was conducted in accordance with the principles of the Declaration of Helsinki. C57BL/6J wild-type (WT) mice were purchased from Jackson Laboratories (USA) and IL-37 transgenic (IL-37tg) mice were homozygous offspring from the original colonies published in Nold et al. (2010) Nature Immunology 11(11):1014-22. Splenocytes were isolated and resuspended in RPMI 1640 (Thermo Fisher Scientific) supplemented with 1% fetal bovine serum (Thermo Fisher Scientific) and 1:500 MycoZap Plus-PR (Lonza) and seeded in a 96-well plate. Cells were then rested for 1 h prior to addition of the anti-IL-37 hybridoma clone supernatants (RF1 and RF3) and incubated for 30 min. Cells were then stimulated overnight with 250 ng/mL LPS. IL-6 was measured in the supernatant by ELISA (BD Bioscience) according to manufacturer's instructions.

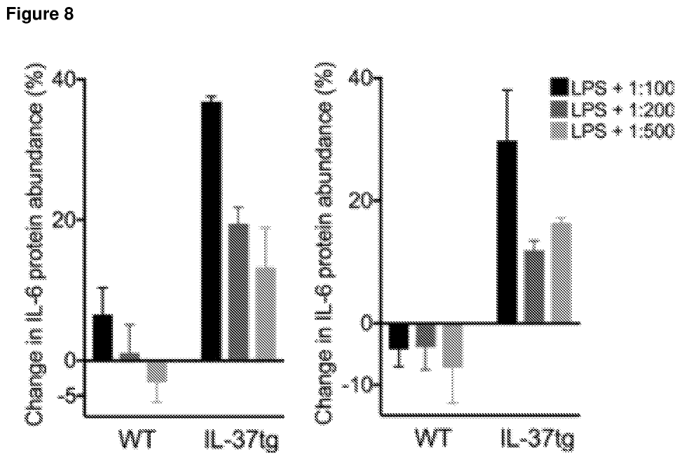

Pre-treatment of mouse splenocytes with anti-IL-37 hybridoma supernatants (clones RF1 and RF3) prior to LPS stimulation resulted in an up to 40% increase of IL-6 abundance compared to LPS alone in IL-37tg splenocytes, whereas the anti-IL-37 hybridoma supernatants had little or no effect in cells from WT mice. Thus, these data demonstrate that the hybridoma supernatants block the anti-inflammatory activity of IL-37 in a specific fashion (FIG. 8).

It will be understood that the invention disclosed and defined in this specification extends to all alternative combinations of two or more of the individual features mentioned or evident from the text or drawings. All of these different combinations constitute various alternative aspects of the invention.

1. An antigen binding site that binds to or specifically binds to IL-37 and inhibits IL-37 activity.

2. An antigen binding site according to claim 1, wherein the antigen binding site comprises an antigen binding domain of an antibody, the antigen binding domain binds to or specifically binds to IL-37 and inhibits IL-37 activity.

3. An antigen binding site according to claim 1 or 2, wherein the IL-37 activity that is inhibited is IL-37 binding to IL-18 receptor α (IL-18Rα), IL-1 receptor 8 or a complex of which comprises IL-18 receptor α (IL-18Rα) and IL-1 receptor 8.

4. An antigen binding site according to any one of claims 1 to 3, wherein the IL-37 activity that is inhibited is IL-37 mediated cell signalling.

5. An antigen binding site according to any one of claims 1 to 4, wherein the IL-37 activity that is inhibited is an IL-37 mediated anti-inflammatory response in vitro or in vivo.

6. An antigen binding site according to any one of claims 1 to 4, wherein the IL-37 activity that is inhibited is an IL-37 mediated innate immune response in vitro or in vivo.

7. An antigen binding site according to any one of claims 1 to 4, wherein the IL-37 activity that is inhibited is an IL-37 mediated adaptive immune response in vitro or in vivo.

8. An antigen binding site according to any one of claims 1 to 7, wherein the IL-37 activity that is inhibited is IL-37 mediated reduction in cytokine production or secretion.

9. An antigen binding site according to claim 8, wherein the cytokine is any one or more selected from the group consisting of sICAM-1, IFN-gamma, I-TAC, G-SCF, IL-16, IL-10, IL-13, TNF, 1-309, IL-2, IL-7, M-CSF, TIMP-1, IL-1alpha, MIP-1alpha, RANTES, MIG, IL-1Ra, sTREM-1, MCP-5, IP-10, MCP-1, IL-23, KC, IL-1beta, MIP-2, IL-17, IL-17F, IL-4, IL-5, IL-33, IL-25, IL-21, IL-22 eotaxin and IL-6.

10. An antigen binding site according to claim 9, wherein the cytokine is a pro-inflammatory cytokine selected from IL-1β, IL-6 and TNF.

11. An antigen binding site according to any one of claims 8 to 10, wherein the IL-37 mediated reduction in cytokine production or secretion is from monocytes, macrophages, peripheral blood mononuclear cells (PBMCs), cancer cells, endothelial, epithelial or dendritic cells or a cell derived therefrom.

12. An antigen binding site according to any one of claims 1 to 11, wherein the antigen binding site binds to or specifically binds to human IL-37.

13. An antigen binding site according to claim 12, wherein the human IL-37 is in dimeric form.

14. An antigen binding site according to claim 12, wherein the human IL-37 is in monomeric form.

15. An antigen binding site according to any one of claims 1 to 14, wherein the antigen binding site binds to or specifically binds to a human IL-37 molecule comprising, consisting essentially of or consisting of an amino acid sequence as shown in SEQ ID NO: 1.

16. An antigen binding site according to any one of claims 1 to 15, wherein the antigen binding site does not detectably bind to or bind significantly to IL-18 and/or IL-1β.

17. An antigen binding site according to any one of claims 1 to 16, wherein the antigen binding site comprises:
FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

18. An antigen binding site according to claim 17, wherein the sequence of any of the complementarity determining regions comprises an amino acid sequence as described in Table 1 herein.

19. An antigen binding site according to claim 17 or 18, wherein the sequence of any of the framework regions comprises an amino acid sequence as described in Table 3 herein.

20. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:5, a CDR2 comprising a sequence at least about 80%, at least 85%, at least 90%, at least 92%, at least 95%, at least 97%, at least 99% identical to a sequence set in SEQ ID NO:6 and a CDR3 comprising a sequence at least about 80%, at least 85%, at least 90%, at least 92%, at least 95%, at least 97%, at least 99% identical to a sequence set forth in SEQ ID NO: 7;
(ii) a V_{H} comprising a sequence at least about 95% or 96% or 97% or 98% or 99% identical to a sequence set forth in SEQ ID NO: 9;
(iii) a V_{L} comprising a CDR1 comprising a sequence at least about 80%, at least 85%, at least 90%, at least 92%, at least 95%, at least 97%, at least 99% identical to a sequence set forth in SEQ ID NO: 2, a CDR2 comprising a sequence at least about 80%, at least 85%, at least 90%, at least 92%, at least 95%, at least 97%, at least 99% identical to a sequence set forth in SEQ ID NO: 3 and a CDR3 comprising a sequence at least about 80%, at least 85%, at least 90%, at least 92%, at least 95%, at least 97%, at least 99% identical to a sequence set forth in SEQ ID NO: 4;
(iv) a V_{L} comprising a sequence at least about 95% identical to a sequence set forth in SEQ ID NO: 8;
(v) a V_{H} comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7;
(vi) a V_{H} comprising a sequence set forth in SEQ ID NO: 9;
(vii) a V_{L} comprising a CDR1 comprising a sequence set SEQ ID NO: 2, a CDR2 comprising a sequence set forth in SEQ ID NO: 3 and a CDR3 comprising a sequence set forth in SEQ ID NO: 4;
(viii) a V_{L} comprising a sequence set forth in SEQ ID NO: 8;
(ix) a V_{H} comprising a CDR1 comprising a sequence set forth in SEQ ID NO: 5, a CDR2 comprising a sequence set forth between in SEQ ID NO: 6 and a CDR3 comprising a sequence set forth in SEQ ID NO: 7; and a V_{L} comprising a CDR1 comprising a sequence set SEQ ID NO: 2, a CDR2 comprising a sequence set forth in SEQ ID NO: 3 and a CDR3 comprising a sequence set forth in SEQ ID NO: 4; and
(x) a V_{H} comprising a sequence set forth in SEQ ID NO: 9 and a V_{L} comprising a sequence set forth in SEQ ID NO: 8.

21. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO: 15, a CDR2 comprising a sequence at least about 80%, at least 85%, at least 90%, at least 92%, at least 95%, at least 97%, at least 99% identical to a sequence set in SEQ ID NO: 16 and a CDR3 comprising a sequence at least about 80%, at least 85%, at least 90%, at least 92%, at least 95%, at least 97%, at least 99% identical to a sequence set forth in SEQ ID NO: 17;
(ii) a V_{H} comprising a sequence at least about 95% or 96% or 97% or 98% or 99% identical to a sequence set forth in SEQ ID NO: 19;
(iii) a V_{L} comprising a CDR1 comprising a sequence at least about 80%, at least 85%, at least 90%, at least 92%, at least 95%, at least 97%, at least 99% identical to a sequence set forth in SEQ ID NO: 12, a CDR2 comprising a sequence at least about 80%, at least 85%, at least 90%, at least 92%, at least 95%, at least 97%, at least 99% identical to a sequence set forth in SEQ ID NO: 13 and a CDR3 comprising a sequence at least about 80%, at least 85%, at least 90%, at least 92%, at least 95%, at least 97%, at least 99% identical to a sequence set forth in SEQ ID NO: 14;
(iv) a V_{L} comprising a sequence at least about 95% identical to a sequence set forth in SEQ ID NO: 18;
(v) a V_{H} comprising a CDR1 comprising a sequence set forth in SEQ ID NO: 15, a CDR2 comprising a sequence set forth between in SEQ ID NO: 16 and a CDR3 comprising a sequence set forth in SEQ ID NO: 17;
(vi) a V_{H} comprising a sequence set forth in SEQ ID NO: 19;
(vii) a V_{L} comprising a CDR1 comprising a sequence set SEQ ID NO: 12, a CDR2 comprising a sequence set forth in SEQ ID NO: 13 and a CDR3 comprising a sequence set forth in SEQ ID NO: 14;
(viii) a V_{L} comprising a sequence set forth in SEQ ID NO: 18;
(ix) a V_{H} comprising a CDR1 comprising a sequence set forth in SEQ ID NO: 15, a CDR2 comprising a sequence set forth between in SEQ ID NO: 16 and a CDR3 comprising a sequence set forth in SEQ ID NO: 17; and a V_{L} comprising a CDR1 comprising a sequence set SEQ ID NO: 12, a CDR2 comprising a sequence set forth in SEQ ID NO: 13 and a CDR3 comprising a sequence set forth in SEQ ID NO: 14; and
(x) a V_{H} comprising a sequence set forth in SEQ ID NO: 19 and a V_{L} comprising a sequence set forth in SEQ ID NO: 18.

22. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:41, a CDR2 comprising a sequence at least about 80%, at least 85%, at least 90%, at least 92%, at least 95%, at least 97%, at least 99% identical to a sequence set in SEQ ID NO:42 and a CDR3 comprising a sequence at least about 80%, at least 85%, at least 90%, at least 92%, at least 95%, at least 97%, at least 99% identical to a sequence set forth in SEQ ID NO: 43;
(ii) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:51, a CDR2 comprising a sequence at least about 80%, at least 85%, at least 90%, at least 92%, at least 95%, at least 97%, at least 99% identical to a sequence set in SEQ ID NO:52 and a CDR3 comprising a sequence at least about 80%, at least 85%, at least 90%, at least 92%, at least 95%, at least 97%, at least 99% identical to a sequence set forth in SEQ ID NO: 53;
(iii) a V_{H} comprising a sequence at least about 95% or 96% or 97% or 98% or 99% identical to a sequence set forth in SEQ ID NO: 45 or 55;
(iv) a V_{L} comprising a CDR1 comprising a sequence at least about 80%, at least 85%, at least 90%, at least 92%, at least 95%, at least 97%, at least 99% identical to a sequence set forth in SEQ ID NO: 38, a CDR2 comprising a sequence at least about 80%, at least 85%, at least 90%, at least 92%, at least 95%, at least 97%, at least 99% identical to a sequence set forth in SEQ ID NO: 39 and a CDR3 comprising a sequence at least about 80%, at least 85%, at least 90%, at least 92%, at least 95%, at least 97%, at least 99% identical to a sequence set forth in SEQ ID NO: 40;
(v) a V_{L} comprising a CDR1 comprising a sequence at least about 80%, at least 85%, at least 90%, at least 92%, at least 95%, at least 97%, at least 99% identical to a sequence set forth in SEQ ID NO: 58, a CDR2 comprising a sequence at least about 80%, at least 85%, at least 90%, at least 92%, at least 95%, at least 97%, at least 99% identical to a sequence set forth in SEQ ID NO: 59 and a CDR3 comprising a sequence at least about 80%, at least 85%, at least 90%, at least 92%, at least 95%, at least 97%, at least 99% identical to a sequence set forth in SEQ ID NO: 60;
(vi) a V_{L} comprising a sequence at least about 95% identical to a sequence set forth in SEQ ID NO: 44 or 64;
(vii) a V_{H} comprising a CDR1 comprising a sequence set forth in SEQ ID NO: 41, a CDR2 comprising a sequence set forth between in SEQ ID NO: 42 and a CDR3 comprising a sequence set forth in SEQ ID NO: 43;
(viii) a V_{H} comprising a CDR1 comprising a sequence set forth in SEQ ID NO: 51, a CDR2 comprising a sequence set forth between in SEQ ID NO: 52 and a CDR3 comprising a sequence set forth in SEQ ID NO: 53;
(ix) a V_{H} comprising a sequence set forth in SEQ ID NO: 45 or 55;
(x) a V_{L} comprising a CDR1 comprising a sequence set SEQ ID NO: 38, a CDR2 comprising a sequence set forth in SEQ ID NO: 39 and a CDR3 comprising a sequence set forth in SEQ ID NO: 40;
(xi) a V_{L} comprising a CDR1 comprising a sequence set SEQ ID NO: 58, a CDR2 comprising a sequence set forth in SEQ ID NO: 59 and a CDR3 comprising a sequence set forth in SEQ ID NO: 60;
(xii) a V_{L} comprising a sequence set forth in SEQ ID NO: 44 or 64;
(xiii) a V_{H} comprising a CDR1 comprising a sequence set forth in SEQ ID NO: 41, a CDR2 comprising a sequence set forth between in SEQ ID NO: 42 and a CDR3 comprising a sequence set forth in SEQ ID NO: 43; and a V_{L} comprising a CDR1 comprising a sequence set SEQ ID NO: 38, a CDR2 comprising a sequence set forth in SEQ ID NO: 39 and a CDR3 comprising a sequence set forth in SEQ ID NO: 40; and
(xiv) a V_{H} comprising a CDR1 comprising a sequence set forth in SEQ ID NO: 51, a CDR2 comprising a sequence set forth between in SEQ ID NO: 52 and a CDR3 comprising a sequence set forth in SEQ ID NO: 53; and a V_{L} comprising a CDR1 comprising a sequence set SEQ ID NO: 48, a CDR2 comprising a sequence set forth in SEQ ID NO: 49 and a CDR3 comprising a sequence set forth in SEQ ID NO: 50; and
(xv) a V_{H} comprising a CDR1 comprising a sequence set forth in SEQ ID NO: 61, a CDR2 comprising a sequence set forth between in SEQ ID NO: 62 and a CDR3 comprising a sequence set forth in SEQ ID NO: 63; and a V_{L} comprising a CDR1 comprising a sequence set SEQ ID NO: 58, a CDR2 comprising a sequence set forth in SEQ ID NO: 59 and a CDR3 comprising a sequence set forth in SEQ ID NO: 60; and
(xvi) a V_{H} comprising a CDR1 comprising a sequence set forth in SEQ ID NO: 71, a CDR2 comprising a sequence set forth between in SEQ ID NO: 72 and a CDR3 comprising a sequence set forth in SEQ ID NO: 73; and a V_{L} comprising a CDR1 comprising a sequence set SEQ ID NO: 68, a CDR2 comprising a sequence set forth in SEQ ID NO: 69 and a CDR3 comprising a sequence set forth in SEQ ID NO: 70; and
(xvii) a V_{H} comprising a sequence set forth in SEQ ID NO: 44 and a V_{L} comprising a sequence set forth in SEQ ID NO: 45;
(xviii) a V_{H} comprising a sequence set forth in SEQ ID NO: 54 and a V_{L} comprising a sequence set forth in SEQ ID NO: 55;
(xix) a V_{H} comprising a sequence set forth in SEQ ID NO: 64 and a V_{L} comprising a sequence set forth in SEQ ID NO: 65;
(xx) a V_{H} comprising a sequence set forth in SEQ ID NO: 74 and a V_{L} comprising a sequence set forth in SEQ ID NO: 75.

23. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:141, a CDR2 comprising a sequence at least about 80%, at least 85%, at least 90%, at least 92%, at least 95%, at least 97%, at least 99% identical to a sequence set in SEQ ID NO:142 and a CDR3 comprising a sequence at least about 80%, at least 85%, at least 90%, at least 92%, at least 95%, at least 97%, at least 99% identical to a sequence set forth in SEQ ID NO: 143;
(ii) a V_{H} comprising a sequence at least about 95% or 96% or 97% or 98% or 99% identical to a sequence set forth in SEQ ID NO: 145;
(iii) a V_{L} comprising a CDR1 comprising a sequence at least about 80%, at least 85%, at least 90%, at least 92%, at least 95%, at least 97%, at least 99% identical to a sequence set forth in SEQ ID NO: 138, a CDR2 comprising a sequence at least about 80%, at least 85%, at least 90%, at least 92%, at least 95%, at least 97%, at least 99% identical to a sequence set forth in SEQ ID NO: 139 and a CDR3 comprising a sequence at least about 80%, at least 85%, at least 90%, at least 92%, at least 95%, at least 97%, at least 99% identical to a sequence set forth in SEQ ID NO: 140;
(iv) a V_{L} comprising a sequence at least about 95% identical to a sequence set forth in SEQ ID NO: 144;
(v) a V_{H} comprising a CDR1 comprising a sequence set forth in SEQ ID NO: 141, a CDR2 comprising a sequence set forth between in SEQ ID NO: 142 and a CDR3 comprising a sequence set forth in SEQ ID NO: 143;
(vi) a V_{H} comprising a sequence set forth in SEQ ID NO: 145;
(vii) a V_{L} comprising a CDR1 comprising a sequence set SEQ ID NO: 138, a CDR2 comprising a sequence set forth in SEQ ID NO: 139 and a CDR3 comprising a sequence set forth in SEQ ID NO: 140;
(viii) a V_{L} comprising a sequence set forth in SEQ ID NO: 144;
(ix) a V_{H} comprising a CDR1 comprising a sequence set forth in SEQ ID NO:141, a CDR2 comprising a sequence set forth between in SEQ ID NO: 142 and a CDR3 comprising a sequence set forth in SEQ ID NO:143; and a V_{L} comprising a CDR1 comprising a sequence set SEQ ID NO: 138, a CDR2 comprising a sequence set forth in SEQ ID NO: 139 and a CDR3 comprising a sequence set forth in SEQ ID NO: 140; and
(x) a V_{H} comprising a sequence set forth in SEQ ID NO: 145 and a V_{L} comprising a sequence set forth in SEQ ID NO: 144.

24. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1 comprising a sequence at least about 80%, at least 85%, at least 90%, at least 92%, at least 95%, at least 97%, at least 99% identical to a sequence set forth in SEQ ID NO:151, a CDR2 comprising a sequence at least about 80%, at least 85%, at least 90%, at least 92%, at least 95%, at least 97%, at least 99% identical to a sequence set in SEQ ID NO:152 and a CDR3 comprising a sequence at least about 80%, at least 85%, at least 90%, at least 92%, at least 95%, at least 97%, at least 99% identical to a sequence set forth in SEQ ID NO: 153;
(ii) a V_{H} comprising a sequence at least about 95% or 96% or 97% or 98% or 99% identical to a sequence set forth in SEQ ID NO: 155;
(iii) a V_{L} comprising a CDR1 comprising a sequence at least about 80%, at least 85%, at least 90%, at least 92%, at least 95%, at least 97%, at least 99% identical to a sequence set forth in SEQ ID NO: 148, a CDR2 comprising a sequence at least about 80%, at least 85%, at least 90%, at least 92%, at least 95%, at least 97%, at least 99% identical to a sequence set forth in SEQ ID NO: 149 and a CDR3 comprising a sequence at least about 80%, at least 85%, at least 90%, at least 92%, at least 95%, at least 97%, at least 99% identical to a sequence set forth in SEQ ID NO: 150;
(iv) a V_{L} comprising a sequence at least about 95% identical to a sequence set forth in SEQ ID NO: 154;
(v) a V_{H} comprising a CDR1 comprising a sequence set forth in SEQ ID NO: 151, a CDR2 comprising a sequence set forth between in SEQ ID NO: 152 and a CDR3 comprising a sequence set forth in SEQ ID NO: 153;
(vi) a V_{H} comprising a sequence set forth in SEQ ID NO: 155;
(vii) a V_{L} comprising a CDR1 comprising a sequence set SEQ ID NO: 148, a CDR2 comprising a sequence set forth in SEQ ID NO: 149 and a CDR3 comprising a sequence set forth in SEQ ID NO: 150;
(viii) a V_{L} comprising a sequence set forth in SEQ ID NO: 154;
(ix) a V_{H} comprising a CDR1 comprising a sequence set forth in SEQ ID NO: 151, a CDR2 comprising a sequence set forth between in SEQ ID NO: 152 and a CDR3 comprising a sequence set forth in SEQ ID NO: 153; and a V_{L} comprising a CDR1 comprising a sequence set SEQ ID NO: 148, a CDR2 comprising a sequence set forth in SEQ ID NO: 149 and a CDR3 comprising a sequence set forth in SEQ ID NO: 150; and
(x) a V_{H} comprising a sequence set forth in SEQ ID NO: 155 and a V_{L} comprising a sequence set forth in SEQ ID NO: 154.

25. An antigen binding site comprising an antigen binding domain of an antibody, wherein the antigen binding domain binds to or specifically binds to IL-37, wherein the antigen binding domain comprises at least one of:
(i) a V_{H} comprising a complementarity determining region (CDR) 1, CDR2 and CDR3 comprising a sequence at least about 80%, at least 85%, at least 90%, at least 92%, at least 95%, at least 97%, at least 99% identical to a sequence set forth in
SEQ ID NO: 5, 6 and 7, respectively;
SEQ ID NO: 15, 16 and 17, respectively;
SEQ ID NO: 41, 42 and 43, respectively;
SEQ ID NO: 51, 52 and 53, respectively;
SEQ ID NO: 61, 62 and 63, respectively;
SEQ ID NO: 71, 72 and 73, respectively;
SEQ ID NO: 81, 82 and 83, respectively;
SEQ ID NO: 91, 92 and 93, respectively;
SEQ ID NO: 101, 102 and 103, respectively;
SEQ ID NO: 111, 112 and 113, respectively;
SEQ ID NO: 121, 122 and 123, respectively;
SEQ ID NO: 131, 132 and 133, respectively;
SEQ ID NO: 141, 142 and 143, respectively;
SEQ ID NO: 151, 152 and 153, respectively;
SEQ ID NO: 161, 162 and 163, respectively;
SEQ ID NO: 171, 172 and 173, respectively;
SEQ ID NO: 181, 182 and 183, respectively;
SEQ ID NO: 191, 192 and 193, respectively; or
SEQ ID NO: 201, 202 and 203, respectively;
(ii) a V_{H} comprising a sequence at least about 95% or 96% or 97% or 98% or 99% identical to a sequence set forth in SEQ ID NO: 9, SEQ ID NO: 19, SEQ ID NO: 45, SEQ ID NO: 55, SEQ ID NO: 65, SEQ ID NO: 75, SEQ ID NO: 85, SEQ ID NO: 95, SEQ ID NO: 105, SEQ ID NO: 115, SEQ ID NO: 125, SEQ ID NO: 135, SEQ ID NO: 145, SEQ ID NO: 155, SEQ ID NO: 165, SEQ ID NO: 175, SEQ ID NO: 185, SEQ ID NO: 195, or SEQ ID NO: 205;
(iii) a V_{L} comprising a CDR1, CDR2 and CDR3 comprising a sequence at least about 80%, at least 85%, at least 90%, at least 92%, at least 95%, at least 97%, at least 99% identical to a sequence set forth in:
SEQ ID NO: 2, 3 and 4, respectively;
SEQ ID NO: 12, 13 and 14, respectively;
SEQ ID NO: 38, 39, and 40, respectively;
SEQ ID NO: 48, 49, and 50, respectively;
SEQ ID NO: 58, 59, and 60 respectively;
SEQ ID NO: 68, 69, and 70 respectively;
SEQ ID NO: 78, 79 and 80 respectively;
SEQ ID NO: 88, 89 and 90, respectively;
SEQ ID NO: 98, 99 and 100 respectively;
SEQ ID NO: 108, 109 and 110, respectively;
SEQ ID NO: 118, 119 and 120, respectively;
SEQ ID NO: 128, 129 and 130, respectively;
SEQ ID NO: 138, 139 and 140, respectively;
SEQ ID NO: 148, 149 and 150 respectively;
SEQ ID NO: 158, 159 and 160 respectively;
SEQ ID NO: 168, 169 and 170, respectively;
SEQ ID NO: 178, 179 and 180, respectively;
SEQ ID NO: 188, 189 and 190, respectively; or
SEQ ID NO: 198, 199 and 200, respectively;
(iv) a V_{L} comprising a sequence at least about 95% or 96% or 97% or 98% or 99% identical to a sequence set forth in identical to a sequence set forth in SEQ ID NO: 8; SEQ ID NO: 18, SEQ ID NO: 44, SEQ ID NO: 54, SEQ ID NO: 64, SEQ ID NO: 74, SEQ ID NO: 84, SEQ ID NO: 94, SEQ ID NO: 104, SEQ ID NO: 114, SEQ ID NO: 124, SEQ ID NO: 134, SEQ ID NO: 144, SEQ ID NO: 154, SEQ ID NO: 164, SEQ ID NO: 174, SEQ ID NO: 184, SEQ ID NO: 194, or SEQ ID NO: 204;
(v) a V_{H} comprising a CDR1, CDR2 and CDR3 comprising a sequence set forth in any one of:
SEQ ID NO: 5, 6 and 7, respectively;
SEQ ID NO: 15, 16 and 17, respectively;
SEQ ID NO: 41, 42 and 43, respectively;
SEQ ID NO: 51, 52 and 53, respectively;
SEQ ID NO: 61, 62 and 63, respectively;
SEQ ID NO: 71, 72 and 73, respectively;
SEQ ID NO: 81, 82 and 83, respectively;
SEQ ID NO: 91, 92 and 93, respectively;
SEQ ID NO: 101, 102 and 103, respectively;
SEQ ID NO: 111, 112 and 113, respectively;
SEQ ID NO: 121, 122 and 123, respectively;
SEQ ID NO: 131, 132 and 133, respectively;
SEQ ID NO: 141, 142 and 143, respectively;
SEQ ID NO: 151, 152 and 153, respectively;
SEQ ID NO: 161, 162 and 163, respectively;
SEQ ID NO: 171, 172 and 173, respectively;
SEQ ID NO: 181, 182 and 183, respectively;
SEQ ID NO: 191, 192 and 193, respectively; or
SEQ ID NO: 201, 202 and 203, respectively;
(vi) a V_{H} comprising a sequence set forth in SEQ ID NO: 9, SEQ ID NO: 19, SEQ ID NO: 45, SEQ ID NO: 55, SEQ ID NO: 65, SEQ ID NO: 75, SEQ ID NO: 85, SEQ ID NO: 95, SEQ ID NO: 105, SEQ ID NO: 115, SEQ ID NO: 125, SEQ ID NO: 135, SEQ ID NO: 145, SEQ ID NO: 155, SEQ ID NO: 165, SEQ ID NO: 175, SEQ ID NO: 185, SEQ ID NO: 195, or SEQ ID NO: 205;
(vii) a V_{L} comprising a CDR1, CDR2 and CDR3 comprising a sequence as set forth in any one of:
SEQ ID NO: 2, 3 and 4, respectively;
SEQ ID NO: 12, 13 and 14, respectively;
SEQ ID NO: 38, 39, and 40, respectively;
SEQ ID NO: 48, 49, and 50, respectively;
SEQ ID NO: 58, 59, and 60 respectively;
SEQ ID NO: 68, 69, and 70 respectively;
SEQ ID NO: 78, 79 and 80 respectively;
SEQ ID NO: 88, 89 and 90, respectively;
SEQ ID NO: 98, 99 and 100 respectively;
SEQ ID NO: 108, 109 and 110, respectively;
SEQ ID NO: 118, 119 and 120, respectively;
SEQ ID NO: 128, 129 and 130, respectively;
SEQ ID NO: 138, 139 and 140, respectively;
SEQ ID NO: 148, 149 and 150 respectively;
SEQ ID NO: 158, 159 and 160 respectively;
SEQ ID NO: 168, 169 and 170, respectively;
SEQ ID NO: 178, 179 and 180, respectively;
SEQ ID NO: 188, 189 and 190, respectively; or
SEQ ID NO: 198, 199 and 200, respectively;
(viii) a V_{L} comprising a sequence set forth in SEQ ID NO: 8, SEQ ID NO: 18, SEQ ID NO: 44, SEQ ID NO: 54, SEQ ID NO: 64, SEQ ID NO: 74, SEQ ID NO: 84, SEQ ID NO: 94, SEQ ID NO: 104, SEQ ID NO: 114, SEQ ID NO: 124, SEQ ID NO: 134, SEQ ID NO: 144, SEQ ID NO: 154, SEQ ID NO: 164, SEQ ID NO: 174, SEQ ID NO: 184, SEQ ID NO: 194, or SEQ ID NO: 204;
(ix) a V_{H} comprising a CDR1, CDR2 and CDR3 and a V_{L} comprising a CDR1, CDR2 and CDR3 comprising the sequences set forth in:
SEQ ID NO: 5, 6, 7 and 2, 3 and 4, respectively;
SEQ ID NO: 15, 16, 17 and 12, 13 and 14, respectively;
SEQ ID NO: 41, 42, 43, and 38, 39 and 40, respectively;
SEQ ID NO: 51, 52, 53, and 48, 49 and 50, respectively;
SEQ ID NO: 61, 62, 63, and 58, 59 and 60, respectively;
SEQ ID NO: 71, 72, 73, and 68 69 and 70, respectively;
SEQ ID NO: 81, 82, 83 and 78, 79 and 80, respectively;
SEQ ID NO: 91, 92, 93, and 88, 89 and 90, respectively;
SEQ ID NO: 101, 102, 103 and 98, 99 and 100, respectively;
SEQ ID NO: 111, 112, 113 and 108, 109, and 110, respectively;
SEQ ID NO: 121, 122, 123 and 118, 119 and 120, respectively;
SEQ ID NO: 131, 132, 133 and 128, 129 and 130, respectively;
SEQ ID NO: 141, 142, 143, and 138, 139 and 140, respectively;
SEQ ID NO: 151, 152, 153, and 148, 149 and 150, respectively;
SEQ ID NO: 161, 162, 163, and 158, 159 and 160, respectively;
SEQ ID NO: 171, 172, 173, and 168, 169 and 170, respectively;
SEQ ID NO: 181, 182, 183, 178, 179 and 180, respectively;
SEQ ID NO: 191, 192, 193, and 188, 189, and 190, respectively; or
SEQ ID NO: 201, 202, 203 and 198, 199 and 200, respectively;
(x) a V_{H} and a V_{L} comprising a sequence set forth in any one of:
SEQ ID NOs: 9 and 8, respectively;
SEQ ID NOs: 19 and 18, respectively;
SEQ ID NOs: 45 and 44, respectively;
SEQ ID NOs: 55 and 54, respectively;
SEQ ID NOs: 65 and 64, respectively;
SEQ ID NOs: 75 and 74, respectively;
SEQ ID NOs: 85 and 84, respectively;
SEQ ID NOs: 95 and 94, respectively;
SEQ ID NOs: 105 and 104, respectively;
SEQ ID NOs: 115 and 114, respectively;
SEQ ID NOs: 125 and 124, respectively;
SEQ ID NOs: 135 and 134, respectively;
SEQ ID NOs: 145 and 144, respectively;
SEQ ID NOs: 155 and 154, respectively;
SEQ ID NOs: 165 and 164, respectively;
SEQ ID NOs: 175 and 174, respectively;
SEQ ID NOs: 185 and 184, respectively;
SEQ ID NOs: 195 and 194, respectively; or
SEQ ID NOs: 205 and 204 respectively.

26. An antigen binding site according to any one of claims 1 to 25, wherein the antigen binding site is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to an immune effector cell.

27. An antigen binding site according to any one of claims 1 to 25, wherein the antigen binding site is in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3;
(viii) one of (i) to (vi) linked to a protein that binds to an immune effector cell.

28. An antigen binding site of any one of claims 1 to 27, wherein the antigen binding site is antibody or antigen binding fragment thereof.

29. A nucleic acid encoding an antigen binding site according to any one of claims 1 to 28.

30. A vector comprising a nucleic acid according to claim 29.

31. A vector according to claim 30, wherein the nucleic acid is operably linked to a promoter.

32. A vector according to claim 31, wherein the vector comprises the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

33. A cell comprising a vector or nucleic acid according to any one of claims 29 to 32.

34. A cell according to claim 33, wherein the cell is a bacterial cell, yeast cell, insect cell or mammalian cell.

35. A pharmaceutical composition comprising an antigen binding site according to any one of claims 1 to 28, and a pharmaceutically acceptable carrier, diluent or excipient.

36. A kit or article of manufacture comprising an antigen binding site according to any one of claims 1 to 28.

37. A method for treating or preventing a condition associated with elevated expression of endogenous IL-37 in a subject, the method comprising administering an antigen binding site according to any one of claims 1 to 28 to the subject, thereby treating or preventing a condition associated with elevated expression of endogenous IL-37.

38. A method for treating or preventing cancer in a subject, the method comprising administering an antigen binding site according to any one of claims 1 to 28 to the subject, thereby treating or preventing cancer.

39. A method for treating or preventing a condition associated with immune paralysis in a subject, the method comprising administering an antigen binding site according to any one of claims 1 to 28 to the subject, thereby treating or preventing a condition associated with immune paralysis.

40. A method according to claim 39, wherein the condition associated with immune paralysis is sepsis, acute liver failure, or chronic liver failure.

41. Use of an antigen binding site according to any one of claims 1 to 28 for determining the levels of IL-37 in a biological sample obtained from an individual.

42. The use of claim 41, wherein determining the levels of IL-37 in the biological sample enables diagnosis of the individual for a disease or condition characterised by altered IL-37 levels.


SEQUENCE LISTING (343 sequences)

SEQ ID NO: 1 - protein, 175 aa, Artificial Sequence
  IL-37 -recombinant (rec) IL-37 (46-218)
GSVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 2 - protein, 10 aa, Artificial Sequence
  Antibody D3 LCDR1
SASSSVSYMH

SEQ ID NO: 3 - protein, 7 aa, Artificial Sequence
  Antibody D3 LCDR2
DTSKLAS

SEQ ID NO: 4 - protein, 9 aa, Artificial Sequence
  Antibody D3 LCDR3
QQWSSNPPT

SEQ ID NO: 5 - protein, 5 aa, Artificial Sequence
  Antibody D3 HCDR1
SYGVH

SEQ ID NO: 6 - protein, 16 aa, Artificial Sequence
  Antibody D3 HCDR2
VIWRGGSTDYNAAFMS

SEQ ID NO: 7 - protein, 10 aa, Artificial Sequence
  Antibody D3 HCDR3
ELWLRRGFAY

SEQ ID NO: 8 - protein, 106 aa, Artificial Sequence
  Antibody D3 VL (FR1-CDR1-FR2-CDR2-FR3-CDR3-FR4)
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLEIK

SEQ ID NO: 9 - protein, 118 aa, Artificial Sequence
  Antibody D3 VH (FR1-CDR1-FR2-CDR2-FR3-CDR3-FR4)
QVQLKQSGPGLVQPSQSLSITCTVSGFSLTSYGVHWVRQSPGKGLEWLGVIWRGGSTDYNAAFMSRLSITKDNSKSQVFFKMNSLQADDTAIYYCAKELWLRRGFAYWGQGTLVTVSA

SEQ ID NO: 10 - DNA, 318 bp, Artificial Sequence
  Antibody D3 VL (DNA)
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccaccgacgttcggtggaggcaccaagctggaaatcaaa

SEQ ID NO: 11 - DNA, 354 bp, Artificial Sequence
  Anitbody D3 VH (DNA)
caggtgcagctgaagcagtcaggacctggcctagtgcagccctcacagagcctgtccataacctgcacagtctctggtttctcattaactagctatggtgtacactgggttcgccagtctccaggaaagggtctggagtggctgggagtgatatggagaggtggaagcacagactacaatgcagctttcatgtccagactgagcatcaccaaggacaactccaagagccaagttttctttaaaatgaacagtctgcaagctgatgacactgccatatactactgtgccaaggagctatggttacgacgagggtttgcttactggggccaagggactctggtcactgtctctgca

SEQ ID NO: 12 - protein, 17 aa, Artificial Sequence
  Antibody C3 LCDR1
KSSQSLLYSSNQKNYLA

SEQ ID NO: 13 - protein, 7 aa, Artificial Sequence
  Antibody C3 LCDR2
WASTRES

SEQ ID NO: 14 - protein, 9 aa, Artificial Sequence
  Antibody C3 LCDR3
QQYYSYPFT

SEQ ID NO: 15 - protein, 5 aa, Artificial Sequence
  Antibody C3 HCDR1
DTYMH

SEQ ID NO: 16 - protein, 17 aa, Artificial Sequence
  Antibody C3 HCDR2
RIDPANGNTKYDPKFQG

SEQ ID NO: 17 - protein, 9 aa, Artificial Sequence
  Antibody C3 HCDR3
SSSPTWFAY

SEQ ID NO: 18 - protein, 113 aa, Artificial Sequence
  Antibody C3 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPFTFGSGTKLEIK

SEQ ID NO: 19 - protein, 118 aa, Artificial Sequence
  Antibody C3 VH
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCAKSSSPTWFAYWGQGTLVTVSA

SEQ ID NO: 20 - DNA, 339 bp, Artificial Sequence
  Antibody C3 VL (DNA)
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccattcacgttcggctcggggacaaagttggaaataaaa

SEQ ID NO: 21 - DNA, 354 bp, Artificial Sequence
  Antibody C3 VH (DNA)
gaggttcagctgcagcagtctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctaagagtagtagccccacctggtttgcttactggggccaagggactctggtcactgtctctgca

SEQ ID NO: 22 - protein, 23 aa, Artificial Sequence
  Antibody C3 LFR1
QIVLTQSPAIMSASPGEKVTMTC

SEQ ID NO: 23 - protein, 15 aa, Artificial Sequence
  Antibody C3 LFR2
WYQQKSGTSPKRWIY

SEQ ID NO: 24 - protein, 32 aa, Artificial Sequence
  Antibody C3 LFR3
GVPARFSGSGSGTSYSLTISSMEAEDAATYYC

SEQ ID NO: 25 - protein, 10 aa, Artificial Sequence
  Antibody C3 LFR4
FGGGTKLEIK

SEQ ID NO: 26 - protein, 30 aa, Artificial Sequence
  Antibody C3 HFR1
QVQLKQSGPGLVQPSQSLSITCTVSGFSLT

SEQ ID NO: 27 - protein, 14 aa, Artificial Sequence
  Antibody C3 HFR2
WVRQSPGKGLEWLG

SEQ ID NO: 28 - protein, 32 aa, Artificial Sequence
  Antibody C3 HFR3
RLSITKDNSKSQVFFKMNSLQADDTAIYYCAK

SEQ ID NO: 29 - protein, 11 aa, Artificial Sequence
  Antibody C3 HFR4
WGQGTLVTVSA

SEQ ID NO: 30 - protein, 23 aa, Artificial Sequence
  Antibody D3 LFR1
DIVMSQSPSSLAVSVGEKVTMSC

SEQ ID NO: 31 - protein, 15 aa, Artificial Sequence
  Antibody D3 LFR2
WYQQKPGQSPKLLIY

SEQ ID NO: 32 - protein, 32 aa, Artificial Sequence
  Antibody D3 LFR3
GVPDRFTGSGSGTDFTLTISSVKAEDLAVYYC

SEQ ID NO: 33 - protein, 10 aa, Artificial Sequence
  Antibody D3 LFR4
FGSGTKLEIK

SEQ ID NO: 34 - protein, 30 aa, Artificial Sequence
  Antibody D3 HFR1
EVQLQQSGAELVKPGASVKLSCTASGFNIK

SEQ ID NO: 35 - protein, 14 aa, Artificial Sequence
  Antibody D3 HFR2
WVKQRPEQGLEWIG

SEQ ID NO: 36 - protein, 32 aa, Artificial Sequence
  Antibody D3 HFR3
KATITADTSSNTAYLQLSSLTSEDTAVYYCAK

SEQ ID NO: 37 - protein, 11 aa, Artificial Sequence
  Antibody D3 HFR4
WGQGTLVTVSA

SEQ ID NO: 38 - protein, 15 aa, Artificial Sequence
  Antibody RD1 (A)  LCDR1
RASESVDIYGNSFMH

SEQ ID NO: 39 - protein, 7 aa, Artificial Sequence
  Antibody RD1 (A) LCDR2
RASNLES

SEQ ID NO: 40 - protein, 9 aa, Artificial Sequence
  Antibody RD1 (A)  LCDR3
QQSYEDPWT

SEQ ID NO: 41 - protein, 5 aa, Artificial Sequence
  Antibody RD1 (A)  HCDR1
NYGMN

SEQ ID NO: 42 - protein, 17 aa, Artificial Sequence
  Antibody RD1 (A)  HCDR2
WINTYIGEPTYADDFKG

SEQ ID NO: 43 - protein, 9 aa, Artificial Sequence
  Antibody RD1 (A) HCDR3
NYGYPWFAY

SEQ ID NO: 44 - protein, 111 aa, Artificial Sequence
  Antibody RD1 (A) VL (FR1-CDR1-FR2-CDR2-FR3- CDR3-FR4)
DIVLTQSPASLAVSLGQRAIISCRASESVDIYGNSFMHWCQQKPGQPPKLLIFRASNLESGIPARFSGSGSRTDFTLTINPVEADDVATYYCQQSYEDPWTFGGGTKLEIK

SEQ ID NO: 45 - protein, 118 aa, Artificial Sequence
  Antibody RD1 (A) VH (FR1-CDR1-FR2-CDR2-FR3- CDR3-FR4)
QIQLVQSGPELKKPGETVKISCKASGYTFKNYGMNWVKQAPGKGLKWMGWINTYIGEPTYADDFKGRFAFSLETSASTAYLQINNLKDEDTATYFCARNYGYPWFAYWGQGTLVTVSA

SEQ ID NO: 46 - DNA, 333 bp, Artificial Sequence
  Antibody RD1 (A) VL(DNA)
gacattgtgctgacccaatctccagcttctttggctgtgtctctagggcagagggccatcatatcctgcagagccagtgaaagtgttgatatttatggcaatagttttatgcactggtgccagcagaagccaggacagccacccaaactcctcatctttcgtgcatccaacctagaatctgggatccctgccaggttcagtggcagtgggtctaggacagacttcaccctcaccattaatcctgtggaggctgatgatgttgcaacctattactgtcaacaaagttatgaggatccgtggacgttcggtggaggcaccaagctggaaatcaaa

SEQ ID NO: 47 - DNA, 354 bp, Artificial Sequence
  Antibody RD1 (A) VH (DNA)
cagatccagttggtgcagtctggacctgagctgaagaagcctggagagacagtcaagatctcctgcaaggcttctgggtataccttcaaaaactatggaatgaactgggtgaagcaggctccaggaaagggtttaaagtggatgggctggataaacacctatattggagagccaacatatgctgatgacttcaagggacggtttgccttctctttggaaacctctgccagcactgcctatttgcagatcaacaacctcaaagatgaggacacggctacatatttctgtgcaagaaactacggctacccctggtttgcttactggggccaagggactctggtcactgtctctgca

SEQ ID NO: 48 - protein, 15 aa, Artificial Sequence
  Antibody RD1 (B) LCDR1
RASESVDIYGNSFMH

SEQ ID NO: 49 - protein, 7 aa, Artificial Sequence
  Antibody RD1 (B) LCDR2
RASNLES

SEQ ID NO: 50 - protein, 7 aa, Artificial Sequence
  Antibody RD1 (B) LCDR3
RASNLES

SEQ ID NO: 51 - protein, 5 aa, Artificial Sequence
  Antibody RD1 (B) HCDR1
DYSMH

SEQ ID NO: 52 - protein, 17 aa, Artificial Sequence
  Antibody RD1 (B) HCDR2
WINTETGEPTYADDFKG

SEQ ID NO: 53 - protein, 8 aa, Artificial Sequence
  Antibody RD1 (B) HCDR3
SYAYAMDY

SEQ ID NO: 54 - protein, 111 aa, Artificial Sequence; identical to SEQ ID NO: 44
  Antibody RD1 (B) VL (FR1-CDR1-FR2-CDR2-FR3- CDR3-FR4)

SEQ ID NO: 55 - protein, 117 aa, Artificial Sequence
  Antibody RD1 (B) VH (FR1-CDR1-FR2-CDR2-FR3- CDR3-FR4)
QIQLVQSGPELEKPGETVKISCKASGYTFTDYSMHWVKQAPGKGLKWMGWINTETGEPTYADDFKGRFAFSLETSASTAYLQINNLKNEDTATYFCARSYAYAMDYWGQGTSVTVSS

SEQ ID NO: 56 - DNA, 333 bp, Artificial Sequence; identical to SEQ ID NO: 46
  Antibody RD1 (B) VL (DNA)

SEQ ID NO: 57 - DNA, 351 bp, Artificial Sequence
  Antibody RD1 (B) VH (DNA)
cagatccagttggtgcagtctggacctgagctggagaagcctggagagacagtcaagatctcctgtaaggcttctggttataccttcacagactattcaatgcactgggtgaagcaggctccaggaaagggtttaaagtggatgggctggataaacactgagactggtgagccaacatatgcagatgacttcaagggacggtttgccttctcgttggaaacctctgccagcactgcctatttgcagatcaacaacctcaaaaatgaggacacggctacatatttctgtgctagatcttacgcctatgctatggactactggggtcaaggaacctcagtcaccgtctcctca

SEQ ID NO: 58 - protein, 11 aa, Artificial Sequence
  Antibody RD1 (C) LCDR1
KASQNVGTNVA

SEQ ID NO: 59 - protein, 7 aa, Artificial Sequence
  Antibody RD1 (C) LCDR2
SASYRYS

SEQ ID NO: 60 - protein, 9 aa, Artificial Sequence
  Antibody RD1 (C) LCDR3
QQYNSYPLT

SEQ ID NO: 61 - protein, 5 aa, Artificial Sequence
  Antibody RD1 (C) HCDR1
NYGMN

SEQ ID NO: 62 - protein, 17 aa, Artificial Sequence
  Antibody RD1 (C) HCDR2
WINTYIGEPTYADDFKG

SEQ ID NO: 63 - protein, 9 aa, Artificial Sequence
  Antibody RD1 (C) HCDR3
NYGYPWFAY

SEQ ID NO: 64 - protein, 107 aa, Artificial Sequence
  Antibody RD1 (C) VL (FR1-CDR1-FR2-CDR2-FR3- CDR3-FR4)
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLELK

SEQ ID NO: 65 - protein, 118 aa, Artificial Sequence; identical to SEQ ID NO: 45
  Antibody RD1 (C) VH (FR1-CDR1-FR2-CDR2-FR3- CDR3-FR4)

SEQ ID NO: 66 - DNA, 321 bp, Artificial Sequence
  Antibody RD1 (C) VL (DNA)
gacattgtcatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgctcacgttcggtgctgggaccaagctggagctgaaa

SEQ ID NO: 67 - DNA, 354 bp, Artificial Sequence; identical to SEQ ID NO: 47
  Antibody RD1 (C) VH (DNA)

SEQ ID NO: 68 - protein, 11 aa, Artificial Sequence
  Antibody RD1 (D) LCDR1
KASQNVGTNVA

SEQ ID NO: 69 - protein, 7 aa, Artificial Sequence
  Antibody RD1 (D) LCDR2
SASYRYS

SEQ ID NO: 70 - protein, 9 aa, Artificial Sequence
  Antibody RD1 (D) LCDR3
QQYNSYPLT

SEQ ID NO: 71 - protein, 5 aa, Artificial Sequence
  Antibody RD1 (D) HCDR1
DYSMH

SEQ ID NO: 72 - protein, 17 aa, Artificial Sequence
  Antibody RD1 (D) HCDR2
WINTETGEPTYADDFKG

SEQ ID NO: 73 - protein, 8 aa, Artificial Sequence
  Antibody RD1 (D) HCDR3
SYAYAMDY

SEQ ID NO: 74 - protein, 107 aa, Artificial Sequence; identical to SEQ ID NO: 64
  Antibody RD1 (D) VL (FR1-CDR1-FR2-CDR2-FR3- CDR3-FR4)

SEQ ID NO: 75 - protein, 117 aa, Artificial Sequence; identical to SEQ ID NO: 55
  Antibody RD1 (D) VH (FR1-CDR1-FR2-CDR2-FR3- CDR3-FR4)

SEQ ID NO: 76 - DNA, 321 bp, Artificial Sequence; identical to SEQ ID NO: 66
  Antibody RD1 (D) VL (DNA)

SEQ ID NO: 77 - DNA, 351 bp, Artificial Sequence; identical to SEQ ID NO: 57
  Antibody RD1 (D) VH (DNA)

SEQ ID NO: 78 - protein, 16 aa, Artificial Sequence
  Antibody RE1 LCDR1
RSSQSLVHSNGNTYLH

SEQ ID NO: 79 - protein, 7 aa, Artificial Sequence
  Antibody RE1 LCDR2
KVSNRFS

SEQ ID NO: 80 - protein, 9 aa, Artificial Sequence
  Antibody RE1 LCDR3
SQCRHVPFT

SEQ ID NO: 81 - protein, 5 aa, Artificial Sequence
  Antibody RE1 HCDR1
DTYMH

SEQ ID NO: 82 - protein, 17 aa, Artificial Sequence
  Antibody RE1 HCDR2
RIDPANGNSIYDPTFQG

SEQ ID NO: 83 - protein, 8 aa, Artificial Sequence
  Antibody RE1 HCDR3
GNWDWFVF

SEQ ID NO: 84 - protein, 112 aa, Artificial Sequence
  Antibody RE1 VL (FR1-CDR1-FR2-CDR2-FR3-CDR3- FR4)
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQCRHVPFTFGSGTKLEIK

SEQ ID NO: 85 - protein, 117 aa, Artificial Sequence
  Antibody RE1 VH (FR1-CDR1-FR2-CDR2-FR3-CDR3- FR4)
EVQLQQSGAELVKPGASVTLSCTASGFNIKDTYMHWVKQRPEQGLEWIGRIDPANGNSIYDPTFQGKATVTADTSSNTAYLQLSSLTSEDTAVYFCSNGNWDWFVFWGQGTLVTVSA

SEQ ID NO: 86 - DNA, 336 bp, Artificial Sequence
  Antibody RE1 VL (DNA)
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaatgtagacatgttccattcacgttcggctcggggacaaagttggaaataaaa

SEQ ID NO: 87 - DNA, 351 bp, Artificial Sequence
  Antibody RE1 VH (DNA)
gaggttcagctgcagcagtctggggcagaacttgtgaagccaggggcctcagtcacgttgtcctgcacagcttctggcttcaacattaaagacacctatatgcactgggtgaagcagaggcctgaacagggcctggagtggattggaaggattgatcctgcgaatggtaattctatatatgacccgacgttccagggcaaggccactgtaacagctgacacatcctccaacacagcctacctgcagctcagcagcctgacatctgaggacactgccgtctatttctgttctaatgggaactgggactggtttgttttctggggccaagggactctggtcactgtctctgca

SEQ ID NO: 88 - protein, 11 aa, Artificial Sequence
  Antibody RB6 LCDR1
KASQNVDTHVA

SEQ ID NO: 89 - protein, 7 aa, Artificial Sequence
  Antibody RB6 LCDR2
SASYRYS

SEQ ID NO: 90 - protein, 9 aa, Artificial Sequence
  Antibody RB6 LCDR3
QQYHSYPLT

SEQ ID NO: 91 - protein, 5 aa, Artificial Sequence
  Antibody RB6 HCDR1
KKGVT

SEQ ID NO: 92 - protein, 16 aa, Artificial Sequence
  Antibody RB6 HCDR2
IIWDDGSINYHSALIS

SEQ ID NO: 93 - protein, 12 aa, Artificial Sequence
  Antibody RB6 HCDR3
RGDVSRGAWFVY

SEQ ID NO: 94 - protein, 107 aa, Artificial Sequence
  Antibody RB6 VL (FR1-CDR1-FR2-CDR2-FR3-CDR3- FR4)
DIVMTQSQEFLSTLVGDRVSVTCKASQNVDTHVAWYQQKPGQSPKTLIYSASYRYSGVPDRFTGSGSGTDFTLTISSVQSEDLAKYFCQQYHSYPLTFGGGTKLELK

SEQ ID NO: 95 - protein, 120 aa, Artificial Sequence
  Antibody RB6 VH (FR1-CDR1-FR2-CDR2-FR3-CDR3- FR4)
QVQLKESGPGLVAPSQSLTITCTVSGFSLTKKGVTWVRQPPGKGLEWLGIIWDDGSINYHSALISRLRITKDNFKSQVFLHLNSLQTDDTATYYCARRGDVSRGAWFVYWGQGTLVTVSA

SEQ ID NO: 96 - DNA, 321 bp, Artificial Sequence
  Antibody RB6 VL (DNA)
gacattgtgatgacccagtctcaagaattcttgtccacattagtaggagacagggtcagcgtcacctgcaaggccagtcagaatgtggatactcatgtagcctggtatcaacagaaaccagggcagtctcctaaaacactgatttactcggcatcttaccggtacagtggagtccctgatcgcttcacaggcagtggatctgggacagatttcactctcaccatcagcagtgtgcagtctgaagacttggcaaagtatttctgtcaacaatatcacagctatcctctcacgttcggaggggggaccaagctggaactgaaa

SEQ ID NO: 97 - DNA, 360 bp, Artificial Sequence
  Antibody RB6 VH (DNA)
caggtgcagttgaaggagtcaggacctggcctggtggcgccctcacagagcctgaccatcacatgcactgtctcagggttctccttaaccaaaaaaggtgtaacgtgggttcgccagcctccaggaaagggtctggagtggctgggaataatatgggatgacgggagcattaattatcactcagctctcatatccagattgcgcatcactaaggataatttcaagagccaagttttcctgcacctgaacagtctgcaaactgatgatacagccacgtattactgtgccagaagaggggacgttagtaggggggcctggtttgtctattggggccaagggactctggtcactgtctctgca

SEQ ID NO: 98 - protein, 15 aa, Artificial Sequence
  Antibody RH1 (A) LCDR1
RASESVDIYGNSFMH

SEQ ID NO: 99 - protein, 7 aa, Artificial Sequence
  Antibody RH1 (A) LCDR2
RASNLES

SEQ ID NO: 100 - protein, 9 aa, Artificial Sequence
  Antibody RH1 (A) LCDR3
QQSYEDPWT

SEQ ID NO: 101 - protein, 5 aa, Artificial Sequence
  Antibody RH1 (A) HCDR1
DYSMH

SEQ ID NO: 102 - protein, 17 aa, Artificial Sequence
  Antibody RH1 (A) HCDR2
WINTETGEPTYADDFKG

SEQ ID NO: 103 - protein, 8 aa, Artificial Sequence
  Antibody RH1 (A) HCDR3
SYAYAMDY

SEQ ID NO: 104 - protein, 111 aa, Artificial Sequence; identical to SEQ ID NO: 44
  Antibody RH1 (A) VL (FR1-CDR1-FR2-CDR2-FR3- CDR3-FR4)

SEQ ID NO: 105 - protein, 117 aa, Artificial Sequence; identical to SEQ ID NO: 55
  Antibody RH1 (A) VH (FR1-CDR1-FR2-CDR2-FR3- CDR3-FR4)

SEQ ID NO: 106 - DNA, 333 bp, Artificial Sequence; identical to SEQ ID NO: 46
  Antibody RH1 (A) VL (DNA)

SEQ ID NO: 107 - DNA, 351 bp, Artificial Sequence; identical to SEQ ID NO: 57
  Antibody RH1 (A) VH (DNA)

SEQ ID NO: 108 - protein, 15 aa, Artificial Sequence
  Antibody RH1 (B) LCDR1
RASESVDIYGNSFMH

SEQ ID NO: 109 - protein, 7 aa, Artificial Sequence
  Antibody RH1 (B) LCDR2
RASNLES

SEQ ID NO: 110 - protein, 9 aa, Artificial Sequence
  Antibody RH1 (B) LCDR3
QQSYEDPWT

SEQ ID NO: 111 - protein, 5 aa, Artificial Sequence
  Antibody RH1 (B) HCDR1
NYGMN

SEQ ID NO: 112 - protein, 17 aa, Artificial Sequence
  Antibody RH1 (B) HCDR2
WINTFTGEPSYADDFKG

SEQ ID NO: 113 - protein, 9 aa, Artificial Sequence
  Antibody RH1 (B) HCDR3
NYGYPWFAY

SEQ ID NO: 114 - protein, 111 aa, Artificial Sequence; identical to SEQ ID NO: 44
  Antibody RH1 (B) VL (FR1-CDR1-FR2-CDR2-FR3- CDR3-FR4)

SEQ ID NO: 115 - protein, 118 aa, Artificial Sequence
  Antibody RH1 (B) VH (FR1-CDR1-FR2-CDR2-FR3- CDR3-FR4)
QIQLVQSGPELKKPGETVKISCKASGYTFTNYGMNWVKQAPGKGLKWMGWINTFTGEPSYADDFKGRFAFSLETSASTAYLQINNLKNEDTATYFCARNYGYPWFAYWGQGTLVTVSA

SEQ ID NO: 116 - DNA, 333 bp, Artificial Sequence; identical to SEQ ID NO: 46
  Antibody RH1 (B) VL (DNA)

SEQ ID NO: 117 - DNA, 354 bp, Artificial Sequence
  Antibody RH1 (B) VH (DNA)
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccttcactggagagccatcatatgctgatgacttcaagggacggtttgccttctctttggaaacctctgccagcactgcctatttgcagatcaacaacctcaaaaatgaggacacggctacatatttctgtgcaagaaactacggctacccctggtttgcttactggggccaagggactctggtcactgtctctgca

SEQ ID NO: 118 - protein, 15 aa, Artificial Sequence
  Antibody RH1 (C) LCDR1
RASKSVSTSGYSYMH

SEQ ID NO: 119 - protein, 7 aa, Artificial Sequence
  Antibody RH1 (C) LCDR2
LVSNLES

SEQ ID NO: 120 - protein, 9 aa, Artificial Sequence
  Antibody RH1 (C) LCDR3
QQSYEDPWT

SEQ ID NO: 121 - protein, 5 aa, Artificial Sequence
  Antibody RH1 (C) HCDR1
DYSMH

SEQ ID NO: 122 - protein, 17 aa, Artificial Sequence
  Antibody RH1 (C) HCDR2
WINTETGEPTYADDFKG

SEQ ID NO: 123 - protein, 8 aa, Artificial Sequence
  Antibody RH1 (C) HCDR3
SYAYAMDY

SEQ ID NO: 124 - protein, 111 aa, Artificial Sequence
  Antibody RH1 (C) VL (FR1-CDR1-FR2-CDR2-FR3- CDR3-FR4)
DIVLTQSPASLAVSLGQRATISYRASKSVSTSGYSYMHWNQQKPGQPPRLLIYLVSNLESGIPARFSGSGSRTDFTLTINPVEADDVATYYCQQSYEDPWTFGGGTKLEIK

SEQ ID NO: 125 - protein, 117 aa, Artificial Sequence; identical to SEQ ID NO: 55
  Antibody RH1 (C) VH (FR1-CDR1-FR2-CDR2-FR3- CDR3-FR4)

SEQ ID NO: 126 - DNA, 333 bp, Artificial Sequence
  Antibody RH1 (C) VL (DNA)
gacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctgggatccctgccaggttcagtggcagtgggtctaggacagacttcaccctcaccattaatcctgtggaggctgatgatgttgcaacctattactgtcaacaaagttatgaggatccgtggacgttcggtggaggcaccaagctggaaatcaaa

SEQ ID NO: 127 - DNA, 351 bp, Artificial Sequence; identical to SEQ ID NO: 57
  Antibody RH1 (C) VH (DNA)

SEQ ID NO: 128 - protein, 15 aa, Artificial Sequence
  Antibody RH1 (D) LCDR1
RASKSVSTSGYSYMH

SEQ ID NO: 129 - protein, 7 aa, Artificial Sequence
  Antibody RH1 (D) LCDR 2
LVSNLES

SEQ ID NO: 130 - protein, 9 aa, Artificial Sequence
  Antibody RH1 (D) LCDR3
QQSYEDPWT

SEQ ID NO: 131 - protein, 5 aa, Artificial Sequence
  Antibody RH1 (D HCDR1
NYGMN

SEQ ID NO: 132 - protein, 17 aa, Artificial Sequence
  Antibody RH1 (D) HCDR2
WINTFTGEPSYADDFKG

SEQ ID NO: 133 - protein, 9 aa, Artificial Sequence
  Antibody RH1 (D) HCDR3
NYGYPWFAY

SEQ ID NO: 134 - protein, 111 aa, Artificial Sequence; identical to SEQ ID NO: 124
  Antibody RH1(D) VL (FR1-CDR1-FR2-CDR2-FR3- CDR3-FR4)

SEQ ID NO: 135 - protein, 118 aa, Artificial Sequence; identical to SEQ ID NO: 115
  Antibody RH1 (D) VH (FR1-CDR1-FR2-CDR2-FR3- CDR3-FR4)

SEQ ID NO: 136 - DNA, 333 bp, Artificial Sequence; identical to SEQ ID NO: 126
  Antibody RH1 (D) VL (DNA)

SEQ ID NO: 137 - DNA, 354 bp, Artificial Sequence; identical to SEQ ID NO: 117
  Antibody RH1 (D) VH (DNA)

SEQ ID NO: 138 - protein, 16 aa, Artificial Sequence
  Antibody RF1 LCDR1
RSSQSLVHSNGDSYLH

SEQ ID NO: 139 - protein, 7 aa, Artificial Sequence
  Antibody RF1 LCDR2
KVSNRLS

SEQ ID NO: 140 - protein, 9 aa, Artificial Sequence
  Antibody RF1 LCDR3
SQSTHVPYT

SEQ ID NO: 141 - protein, 5 aa, Artificial Sequence
  Antibody RF1 HCDR1
NYWLH

SEQ ID NO: 142 - protein, 17 aa, Artificial Sequence
  Antibody RF1 HCDR2
MIDPSDSFTTYNQKFKG

SEQ ID NO: 143 - protein, 3 aa, Artificial Sequence
  Antibody RF1 HCDR3
VDH

SEQ ID NO: 144 - protein, 112 aa, Artificial Sequence
  Antibody RF1 VL (FR1-CDR1-FR2-CDR2-FR3-CDR3- FR4)
DVVMTQTPLSLPVSLGDQASMSCRSSQSLVHSNGDSYLHWYLQKPGQSPKLLIYKVSNRLSGVPDRFSGSGSGTDFTLKISRVEAEDLGIYFCSQSTHVPYTFGGGTKLEIK

SEQ ID NO: 145 - protein, 112 aa, Artificial Sequence
  Antibody RF1 VH (FR1-CDR1-FR2-CDR2-FR3-CDR3- FR4)
QVQLQQPGAELVKPGASVKMSCKASGYTFTNYWLHWVMQRPGQGLEWIGMIDPSDSFTTYNQKFKGKATLTVDTSSSTAFMQLSSLTSEDSAVYYCTTVDHWGRGTTLTVSS

SEQ ID NO: 146 - DNA, 336 bp, Artificial Sequence
  Antibody RF1 VL (DNA)
gatgttgtgatgacccaaactccactctccctgcctgtcagtcttggagatcaagcctccatgtcttgcagatctagtcagagccttgtacacagtaatggagactcctatttacattggtacctgcagaagccaggccagtctccaaagctcctgatctacaaagtttccaaccgattgtctggggtcccagacaggttcagtggcagtggatcagggacagatttcacactcaagatcagcagagtggaggctgaggatctgggaatttatttctgctctcaaagtacacatgttccgtacacgttcggaggggggaccaagctggaaataaaa

SEQ ID NO: 147 - DNA, 336 bp, Artificial Sequence
  Antibody RF1 VH (DNA)
caggtccaactgcagcagcctggggctgagctggtgaagcctggggcttcagtgaagatgtcctgcaaggcttctggctacaccttcaccaactactggttgcactgggtgatgcagaggcctggacaaggccttgagtggatcggaatgattgatccttctgatagttttactacctacaatcaaaagttcaagggcaaggccacattgactgtagacacatcctccagcacagccttcatgcagctcagcagcctgacatctgaagactctgcggtctattattgtactacggtggaccactggggccgaggcaccactctcacagtctcctca

SEQ ID NO: 148 - protein, 16 aa, Artificial Sequence
  Antibody RF3 LCDR1
RSSQSLVHSNGNTYLH

SEQ ID NO: 149 - protein, 7 aa, Artificial Sequence
  Antibody RF3 LCDR2
IVSNRFS

SEQ ID NO: 150 - protein, 9 aa, Artificial Sequence
  Antibody RF3 LCDR3
SQSTHIPYT

SEQ ID NO: 151 - protein, 5 aa, Artificial Sequence
  Antibody RF3 HCDR1
NYWMN

SEQ ID NO: 152 - protein, 17 aa, Artificial Sequence
  Antibody RF3 HCDR2
MIDPSDSETHYNQVFKD

SEQ ID NO: 153 - protein, 3 aa, Artificial Sequence
  Antibody RF3 HCDR3
DFG

SEQ ID NO: 154 - protein, 112 aa, Artificial Sequence
  Antibody RF3 VL (FR1-CDR1-FR2-CDR2-FR3-CDR3- FR4)
DVVMTQTPLSLPVSLGDQASISCRSSQSLVHSNGNTYLHWYLQKPGQSPKLLIYIVSNRFSGVPDRFGGSGSGTDFTLKISRVEAEDLGVYFCSQSTHIPYTFGGGTKLEMK

SEQ ID NO: 155 - protein, 112 aa, Artificial Sequence
  Antibody RF3 VH (FR1-CDR1-FR2-CDR2-FR3-CDR3- FR4)
QVQLQQPGAELVRPGASVKLSCKASGYTFTNYWMNWWKQRPGQGLEWIGMIDPSDSETHYNQVFKDKATLTVDISSTTAYMHLSSLTSEDSAVYYCARDFGWGQGTLVTVSA

SEQ ID NO: 156 - DNA, 336 bp, Artificial Sequence
  Antibody RF3 VL (DNA)
gatgttgtgatgacccaaactccactctccctgcctgtcagtcttggagatcaagcctccatctcttgcagatctagtcagagccttgtacacagtaatggaaacacctatttacactggtacctgcagaagccaggccagtctccaaagctcctgatctacatagtttccaaccgattttctggggtcccagacaggttcggtggcagtggatcagggacagatttcacactcaagatcagcagagtggaggctgaggatctgggagtttatttctgctctcaaagtacacatattccgtacacgttcggaggggggaccaagctggaaatgaaa

SEQ ID NO: 157 - DNA, 336 bp, Artificial Sequence
  Antibody RF3 VH (DNA)
caggtccaactgcagcagcctggggctgagctggtgaggcctggggcttcagtgaagctgtcttgcaaggcttctggctacaccttcaccaattactggatgaactggtggaaacagaggcctggacaaggccttgaatggattggtatgattgatccttcagacagtgaaactcactacaatcaagtgttcaaggacaaggccacattgactgtagacatatcctccaccacagcctacatgcacctcagcagcctgacatctgaggactctgcggtctattactgtgcaagagacttcggctggggccaagggactctggtcactgtctctgca

SEQ ID NO: 158 - protein, 16 aa, Artificial Sequence
  Antibody RA5 (A) LCDR1
RSSQSIVHSNGNTYLE

SEQ ID NO: 159 - protein, 7 aa, Artificial Sequence
  Antibody RA5 (A) LCDR2
KVSNRFS

SEQ ID NO: 160 - protein, 9 aa, Artificial Sequence
  Antibody RA5 (A) LCDR3
FQGSHVPWT

SEQ ID NO: 161 - protein, 5 aa, Artificial Sequence
  Antibody RA5 (A) HCDR1
NYLIE

SEQ ID NO: 162 - protein, 17 aa, Artificial Sequence
  Antibody RA5 (A) HCDR2
VINPGSGGTNYNEKFKG

SEQ ID NO: 163 - protein, 5 aa, Artificial Sequence
  Antibody RA5 (A) HCDR3
DDSAY

SEQ ID NO: 164 - protein, 112 aa, Artificial Sequence
  Antibody RA5 (A) VL (FR1-CDR1-FR2-CDR2-FR3- CDR3-FR4)
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 165 - protein, 114 aa, Artificial Sequence
  Antibody RA5 (A) VH (FR1-CDR1-FR2-CDR2-FR3- CDR3-FR4)
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDDSAYWGQGTLVTVSA

SEQ ID NO: 166 - DNA, 336 bp, Artificial Sequence
  Antibody RA5 (A) VL (DNA)
gatgttttgatgacccaaactccactctccctgcctgtcagtcttggagatcaagcctccatctcttgcagatctagtcagagtattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgttttcaaggttcacatgttccgtggacgttcggtggaggcaccaagctggaaatcaaa

SEQ ID NO: 167 - DNA, 342 bp, Artificial Sequence
  Antibody RA5 (A) VH (DNA)
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagagacgacagtgcttactggggccaagggactctggtcactgtctctgca

SEQ ID NO: 168 - protein, 11 aa, Artificial Sequence
  Antibody RA5 (B) LCDR1
RASQSISNNLH

SEQ ID NO: 169 - protein, 7 aa, Artificial Sequence
  Antibody RA5 (B) LCDR2
YASQSIS

SEQ ID NO: 170 - protein, 9 aa, Artificial Sequence
  Antibody RA5 (B) LCDR3
QQSNSWPLT

SEQ ID NO: 171 - protein, 5 aa, Artificial Sequence
  Antibody RA5 (B) HCDR1
NYLIE

SEQ ID NO: 172 - protein, 17 aa, Artificial Sequence
  Antibody RA5 (B) HCDR2
VINPGSGGTNYNEKFKG

SEQ ID NO: 173 - protein, 5 aa, Artificial Sequence
  Antibody RA5 (B) HCDR3
DDSAY

SEQ ID NO: 174 - protein, 107 aa, Artificial Sequence
  Antibody RA5 (B) VL (FR1-CDR1-FR2-CDR2-FR3- CDR3-FR4)
DIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 175 - protein, 114 aa, Artificial Sequence; identical to SEQ ID NO: 165
  Antibody RA5 (B) VH (FR1-CDR1-FR2-CDR2-FR3- CDR3-FR4)

SEQ ID NO: 176 - DNA, 321 bp, Artificial Sequence
  Antibody RA5 (B) VL (DNA)
gatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggcctctcacgttcggtgctgggaccaagctggagctgaaa

SEQ ID NO: 177 - DNA, 342 bp, Artificial Sequence; identical to SEQ ID NO: 167
  Antibody RA5 (B) VH (DNA)

SEQ ID NO: 178 - protein, 16 aa, Artificial Sequence
  Antibody RD3 LCDR1
RSSQSIVHSNGNTYLE

SEQ ID NO: 179 - protein, 7 aa, Artificial Sequence
  Antibody RD3 LCDR2
KVSNRFS

SEQ ID NO: 180 - protein, 9 aa, Artificial Sequence
  Antibody RD3 LCDR3
FQGSHVPWT

SEQ ID NO: 181 - protein, 5 aa, Artificial Sequence
  Antibody RD3 HCDR1
SYWMH

SEQ ID NO: 182 - protein, 17 aa, Artificial Sequence
  Antibody RD3 HCDR2
EIDPSDSYTNYNQKFKG

SEQ ID NO: 183 - protein, 10 aa, Artificial Sequence
  Antibody RD3 HCDR3
MITTQGYFDY

SEQ ID NO: 184 - protein, 112 aa, Artificial Sequence; identical to SEQ ID NO: 164
  Antibody RD3 VL (FR1-CDR1-FR2-CDR2-FR3- CDR3-FR4)

SEQ ID NO: 185 - protein, 119 aa, Artificial Sequence
  Antibody RD3 VH (FR1-CDR1-FR2-CDR2-FR3- CDR3-FR4)
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCANMITTQGYFDYWGQGTTLTVSS

SEQ ID NO: 186 - DNA, 336 bp, Artificial Sequence
  Antibody RD3 VL (DNA)
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 187 - DNA, 357 bp, Artificial Sequence
  Antibody RD3 VH (DNA)
caggtccaactgcagcagcctggggctgagcttgtgaagcctggggcttcagtgaagctgtcctgcaaggcttctggctacaccttcaccagctactggatgcactgggtgaagcagaggcctggacaaggccttgagtggatcggagagattgatccttctgatagttatactaactacaatcaaaagttcaagggcaaggccacattgactgtagacaaatcctccagcacagcctacatgcagctcagcagcctgacatctgaggactctgcggtctattactgtgcaaatatgattacgacgcagggctactttgactactggggccaaggcaccactctcacagtctcctca

SEQ ID NO: 188 - protein, 16 aa, Artificial Sequence
  Antibody RC1 LCDR1
RSSLSLVYSNGNTYLH

SEQ ID NO: 189 - protein, 7 aa, Artificial Sequence
  Antibody RC1 LCDR2
KVSNRFS

SEQ ID NO: 190 - protein, 9 aa, Artificial Sequence
  Antibody RC1 LCDR3
SQNTHVPFT

SEQ ID NO: 191 - protein, 5 aa, Artificial Sequence
  Antibody RC1 HCDR1
DTYIH

SEQ ID NO: 192 - protein, 17 aa, Artificial Sequence
  Antibody RC1 HCDR2
RIDPANGNTKYDPKFQD

SEQ ID NO: 193 - protein, 8 aa, Artificial Sequence
  Antibody RC1 HCDR3
GNWDWFAY

SEQ ID NO: 194 - protein, 112 aa, Artificial Sequence
  Antibody RC1 VL (FR1-CDR1-FR2-CDR2-FR3- CDR3-FR4)
DVVMTQTPLSLPVSLGDQASISCRSSLSLVYSNGNTYLHWYQQKPGQSPKLLIFKVSNRFSGVPDRFSGSGSGTDFTLKISRVEAEDLGVYFCSQNTHVPFTFGSGTKLEIK

SEQ ID NO: 195 - protein, 117 aa, Artificial Sequence
  Antibody RC1 VH (FR1-CDR1-FR2-CDR2-FR3- CDR3-FR4)
EVQLQQSGAELVKPGASVKLSCTASGLNIKDTYIHWVKQRPEQGLEWIGRIDPANGNTKYDPKFQDKATITADTSSNTAYLHLSSLTSEDTAVYYCTNGNWDWFAYWGQGTLVTVSA

SEQ ID NO: 196 - DNA, 336 bp, Artificial Sequence
  Antibody RC1 VL (DNA)
gatgttgtgatgacccaaactccactctccctgcctgtcagtcttggagatcaagcctccatctcttgtagatctagtctgagccttgtatacagtaatggaaacacctatttacattggtaccagcagaagccaggccagtctccaaagctcctgatcttcaaagtttccaaccgattttctggggtcccagacaggttcagtggcagtggatcagggacagatttcacactcaagatcagcagagtggaggctgaggatctgggagtttatttctgctctcaaaatacacatgttccattcacgttcggctcggggacaaagttggaaataaaa

SEQ ID NO: 197 - DNA, 351 bp, Artificial Sequence
  Antibody RC1 VH (DNA)
gaggttcagctgcagcagtctggggcagagcttgtgaagccaggggcctcagtcaagttgtcctgcacagcttctggcctcaacattaaagacacctatatacactgggtgaagcagaggcctgaacagggcctggagtggattggaaggattgatcctgcgaatggaaatactaaatatgacccgaagttccaggacaaggccactataacagcagacacatcctccaacacagcctacctgcacctcagcagcctgacatctgaggacactgccgtctattactgtactaatgggaactgggactggtttgcttactggggccaagggactctggtcactgtctctgca

SEQ ID NO: 198 - protein, 16 aa, Artificial Sequence
  Antibody RF2 LCDR1
KSGQSLVHSNGNTYLR

SEQ ID NO: 199 - protein, 7 aa, Artificial Sequence
  Antibody RF2 LCDR2
KVSNRFS

SEQ ID NO: 200 - protein, 9 aa, Artificial Sequence
  Antibody RF2 LCDR3
SQSTHVPLT

SEQ ID NO: 201 - protein, 5 aa, Artificial Sequence
  Antibody RF2 HCDR1
DYYMH

SEQ ID NO: 202 - protein, 17 aa, Artificial Sequence
  Antibody RF2 HCDR2
WIDPENGNTIYDPKFQG

SEQ ID NO: 203 - protein, 3 aa, Artificial Sequence
  Antibody RF2 HCDR3
EDY

SEQ ID NO: 204 - protein, 112 aa, Artificial Sequence
  Antibody RF2 VL (FR1-CDR1-FR2-CDR2-FR3- CDR3-FR4)
DIVMTQTPLSLPVSLGDQASISCKSGQSLVHSNGNTYLRWYLQKPGQSPKLLIYKVSNRFSGVPDRFSGSGSGTDFTLKISRVEAEDLGVYFCSQSTHVPLTFGAGTKLELK

SEQ ID NO: 205 - protein, 112 aa, Artificial Sequence
  Antibody RF2 VH (FR1-CDR1-FR2-CDR2-FR3- CDR3-FR4)
EVQLQQSGAELVRPGALVKLSSKASGFNIKDYYMHWVKQRPEQGLEWIGWIDPENGNTIYDPKFQGKATITADTSSNTTYLQLSSLTSEDTAVYYCTREDYWGQGTSVTVSS

SEQ ID NO: 206 - DNA, 336 bp, Artificial Sequence
  Antibody RF2 VL (DNA)
gatattgtgatgacccaaactccactctccctgcctgtcagtcttggagatcaagcctccatctcttgcaaatctggtcagagccttgtacacagtaatggaaacacctatttacgttggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaa

SEQ ID NO: 207 - DNA, 336 bp, Artificial Sequence
  Antibody RF2 VH (DNA)
gaggttcagctgcagcagtctggggctgagcttgtgaggccaggggccttagtcaagttgtcctccaaagcttctggcttcaacattaaagactactatatgcactgggtgaagcagaggcctgaacagggcctggagtggattggatggattgatcctgagaatggtaatactatatatgacccgaagttccagggcaaggccactataacagcagacacatcctccaacacaacctacctccagctcagcagcctgacatctgaggacactgccgtctattactgtactcgagaggactactggggtcaaggaacctcagtcaccgtctcctca

SEQ ID NO: 208 - protein, 23 aa, Artificial Sequence
  Antibody RD1 (A) LFR1
DIVLTQSPASLAVSLGQRAIISC

SEQ ID NO: 209 - protein, 15 aa, Artificial Sequence
  Antibody RD1 (A) LFR2
WCQQKPGQPPKLLIF

SEQ ID NO: 210 - protein, 32 aa, Artificial Sequence
  Antibody RD1 (A) LFR3
GIPARFSGSGSRTDFTLTINPVEADDVATYYC

SEQ ID NO: 211 - protein, 10 aa, Artificial Sequence
  Antibody RD1 (A) LFR4
FGGGTKLEIK

SEQ ID NO: 212 - protein, 30 aa, Artificial Sequence
  Antibody RD1 (A) HFR1
QIQLVQSGPELKKPGETVKISCKASGYTFK

SEQ ID NO: 213 - protein, 14 aa, Artificial Sequence
  Antibody RD1 (A) HFR2
WVKQAPGKGLKWMG

SEQ ID NO: 214 - protein, 32 aa, Artificial Sequence
  Antibody RD1 (A) HFR3
RFAFSLETSASTAYLQINNLKDEDTATYFCAR

SEQ ID NO: 215 - protein, 11 aa, Artificial Sequence
  Antibody RD1 (A) HFR4
WGQGTLVTVSA

SEQ ID NO: 216 - protein, 23 aa, Artificial Sequence
  Antibody RD1 (B) LFR1
DIVLTQSPASLAVSLGQRAIISC

SEQ ID NO: 217 - protein, 15 aa, Artificial Sequence
  Antibody RD1 (B) LFR2
WCQQKPGQPPKLLIF

SEQ ID NO: 218 - protein, 32 aa, Artificial Sequence; identical to SEQ ID NO: 210
  Antibody RD1 (B) LFR3

SEQ ID NO: 219 - protein, 10 aa, Artificial Sequence
  Antibody RD1 (B) LFR4
FGGGTKLEIK

SEQ ID NO: 220 - protein, 30 aa, Artificial Sequence
  Antibody RD1 (B) HFR1
QIQLVQSGPELEKPGETVKISCKASGYTFT

SEQ ID NO: 221 - protein, 14 aa, Artificial Sequence
  Antibody RD1 (B) HFR2
WVKQAPGKGLKWMG

SEQ ID NO: 222 - protein, 32 aa, Artificial Sequence
  Antibody RD1 (B) HFR3
RFAFSLETSASTAYLQINNLKNEDTATYFCAR

SEQ ID NO: 223 - protein, 11 aa, Artificial Sequence
  Antibody RD1 (B) HFR4
WGQGTSVTVSS

SEQ ID NO: 224 - protein, 23 aa, Artificial Sequence
  Antibody  RD1 (C) LFR1
DIVMTQSQKFMSTSVGDRVSVTC

SEQ ID NO: 225 - protein, 15 aa, Artificial Sequence
  Antibody  RD1 (C) LFR2
WYQQKPGQSPKALIY

SEQ ID NO: 226 - protein, 32 aa, Artificial Sequence
  Antibody  RD1 (C) LFR3
GVPDRFTGSGSGTDFTLTISNVQSEDLAEYFC

SEQ ID NO: 227 - protein, 10 aa, Artificial Sequence
  Antibody  RD1 (C) LFR4
FGAGTKLELK

SEQ ID NO: 228 - protein, 30 aa, Artificial Sequence; identical to SEQ ID NO: 212
  Antibody  RD1 (C) HFR1

SEQ ID NO: 229 - protein, 14 aa, Artificial Sequence
  Antibody  RD1 (C) HFR2
WVKQAPGKGLKWMG

SEQ ID NO: 230 - protein, 32 aa, Artificial Sequence; identical to SEQ ID NO: 214
  Antibody  RD1 (C) HFR3

SEQ ID NO: 231 - protein, 11 aa, Artificial Sequence
  Antibody  RD1 (C) HFR4
WGQGTLVTVSA

SEQ ID NO: 232 - protein, 23 aa, Artificial Sequence
  Antibody RD1 (D) LFR1
DIVMTQSQKFMSTSVGDRVSVTC

SEQ ID NO: 233 - protein, 15 aa, Artificial Sequence
  Antibody RD1 (D) LFR2
WYQQKPGQSPKALIY

SEQ ID NO: 234 - protein, 32 aa, Artificial Sequence; identical to SEQ ID NO: 226
  Antibody RD1 (D) LFR3

SEQ ID NO: 235 - protein, 10 aa, Artificial Sequence
  Antibody RD1 (D) LFR4
FGAGTKLELK

SEQ ID NO: 236 - protein, 30 aa, Artificial Sequence; identical to SEQ ID NO: 220
  Antibody RD1 (D) HFR1

SEQ ID NO: 237 - protein, 14 aa, Artificial Sequence
  Antibody RD1 (D) HFR2
WVKQAPGKGLKWMG

SEQ ID NO: 238 - protein, 32 aa, Artificial Sequence; identical to SEQ ID NO: 222
  Antibody RD1 (D) HFR3

SEQ ID NO: 239 - protein, 11 aa, Artificial Sequence
  Antibody RD1 (D) HFR4
WGQGTSVTVSS

SEQ ID NO: 240 - protein, 23 aa, Artificial Sequence
  Antibody RE1 LFR1
DVVMTQTPLSLPVSLGDQASISC

SEQ ID NO: 241 - protein, 15 aa, Artificial Sequence
  Antibody RE1 LFR2
WYLQKPGQSPKLLIY

SEQ ID NO: 242 - protein, 32 aa, Artificial Sequence
  Antibody RE1 LFR3
GVPDRFSGSGSGTDFTLKISRVEAEDLGVYFC

SEQ ID NO: 243 - protein, 10 aa, Artificial Sequence
  Antibody RE1 LFR4
FGSGTKLEIK

SEQ ID NO: 244 - protein, 30 aa, Artificial Sequence
  Antibody RE1 HFR1
EVQLQQSGAELVKPGASVTLSCTASGFNIK

SEQ ID NO: 245 - protein, 14 aa, Artificial Sequence
  Antibody RE1 HFR2
WVKQRPEQGLEWIG

SEQ ID NO: 246 - protein, 32 aa, Artificial Sequence
  Antibody RE1 HFR3
KATVTADTSSNTAYLQLSSLTSEDTAVYFCSN

SEQ ID NO: 247 - protein, 11 aa, Artificial Sequence
  Antibody RE1 HFR4
WGQGTLVTVSA

SEQ ID NO: 248 - protein, 23 aa, Artificial Sequence
  Antibody RB6 LFR1
DIVMTQSQEFLSTLVGDRVSVTC

SEQ ID NO: 249 - protein, 15 aa, Artificial Sequence
  Antibody RB6 LFR2
WYQQKPGQSPKTLIY

SEQ ID NO: 250 - protein, 32 aa, Artificial Sequence
  Antibody RB6 LFR3
GVPDRFTGSGSGTDFTLTISSVQSEDLAKYFC

SEQ ID NO: 251 - protein, 10 aa, Artificial Sequence
  Antibody RB6 LFR4
FGGGTKLELK

SEQ ID NO: 252 - protein, 30 aa, Artificial Sequence
  Antibody RB6 HFR1
QVQLKESGPGLVAPSQSLTITCTVSGFSLT

SEQ ID NO: 253 - protein, 14 aa, Artificial Sequence
  Antibody RB6 HFR2
WVRQPPGKGLEWLG

SEQ ID NO: 254 - protein, 32 aa, Artificial Sequence
  Antibody RB6 HFR3
RLRITKDNFKSQVFLHLNSLQTDDTATYYCAR

SEQ ID NO: 255 - protein, 11 aa, Artificial Sequence
  Antibody RB6 HFR4
WGQGTLVTVSA

SEQ ID NO: 256 - protein, 23 aa, Artificial Sequence
  Antibody RH1 (A) LFR1
DIVLTQSPASLAVSLGQRAIISC

SEQ ID NO: 257 - protein, 15 aa, Artificial Sequence
  Antibody RH1 (A) LFR2
WCQQKPGQPPKLLIF

SEQ ID NO: 258 - protein, 32 aa, Artificial Sequence; identical to SEQ ID NO: 210
  Antibody RH1 (A) LFR3

SEQ ID NO: 259 - protein, 10 aa, Artificial Sequence
  Antibody RH1 (A) LFR4
FGGGTKLEIK

SEQ ID NO: 260 - protein, 30 aa, Artificial Sequence; identical to SEQ ID NO: 220
  Antibody RH1 (A) HFR1

SEQ ID NO: 261 - protein, 14 aa, Artificial Sequence
  Antibody RH1 (A) HFR2
WVKQAPGKGLKWMG

SEQ ID NO: 262 - protein, 32 aa, Artificial Sequence; identical to SEQ ID NO: 222
  Antibody RH1 (A) HFR3

SEQ ID NO: 263 - protein, 11 aa, Artificial Sequence
  Antibody RH1 (A) HFR4
WGQGTSVTVSS

SEQ ID NO: 264 - protein, 23 aa, Artificial Sequence
  Antibody RH1 (B) LFR1
DIVLTQSPASLAVSLGQRAIISC

SEQ ID NO: 265 - protein, 15 aa, Artificial Sequence
  Antibody RH1 (B) LFR2
WCQQKPGQPPKLLIF

SEQ ID NO: 266 - protein, 32 aa, Artificial Sequence; identical to SEQ ID NO: 210
  Antibody RH1 (B) LFR3

SEQ ID NO: 267 - protein, 10 aa, Artificial Sequence
  Antibody RH1 (B) LFR4
FGGGTKLEIK

SEQ ID NO: 268 - protein, 30 aa, Artificial Sequence
  Antibody RH1 (B) HFR1
QIQLVQSGPELKKPGETVKISCKASGYTFT

SEQ ID NO: 269 - protein, 14 aa, Artificial Sequence
  Antibody RH1 (B) HFR2
WVKQAPGKGLKWMG

SEQ ID NO: 270 - protein, 32 aa, Artificial Sequence; identical to SEQ ID NO: 222
  Antibody RH1 (B) HFR3

SEQ ID NO: 271 - protein, 11 aa, Artificial Sequence
  Antibody RH1 (B) HFR4
WGQGTLVTVSA

SEQ ID NO: 272 - protein, 23 aa, Artificial Sequence
  Antibody RH1 (C) LFR1
DIVLTQSPASLAVSLGQRATISY

SEQ ID NO: 273 - protein, 15 aa, Artificial Sequence
  Antibody RH1 (C) LFR2
WNQQKPGQPPRLLIY

SEQ ID NO: 274 - protein, 32 aa, Artificial Sequence; identical to SEQ ID NO: 210
  Antibody RH1 (C) LFR3

SEQ ID NO: 275 - protein, 10 aa, Artificial Sequence
  Antibody RH1 (C) LFR4
FGGGTKLEIK

SEQ ID NO: 276 - protein, 30 aa, Artificial Sequence; identical to SEQ ID NO: 220
  Antibody RH1 (C) HFR1

SEQ ID NO: 277 - protein, 14 aa, Artificial Sequence
  Antibody RH1 (C) HFR2
WVKQAPGKGLKWMG

SEQ ID NO: 278 - protein, 32 aa, Artificial Sequence; identical to SEQ ID NO: 222
  Antibody RH1 (C) HFR3

SEQ ID NO: 279 - protein, 11 aa, Artificial Sequence
  Antibody RH1 (C) HFR4
WGQGTSVTVSS

SEQ ID NO: 280 - protein, 23 aa, Artificial Sequence
  Antibody RH1 (D) LFR1
DIVLTQSPASLAVSLGQRATISY

SEQ ID NO: 281 - protein, 15 aa, Artificial Sequence
  Antibody RH1 (D) LFR2
WNQQKPGQPPRLLIY

SEQ ID NO: 282 - protein, 32 aa, Artificial Sequence; identical to SEQ ID NO: 210
  Antibody RH1 (D) LFR3

SEQ ID NO: 283 - protein, 10 aa, Artificial Sequence
  Antibody RH1 (D) LFR4
FGGGTKLEIK

SEQ ID NO: 284 - protein, 30 aa, Artificial Sequence; identical to SEQ ID NO: 268
  Antibody RH1 (D) HFR1

SEQ ID NO: 285 - protein, 14 aa, Artificial Sequence
  Antibody RH1 (D) HFR2
WVKQAPGKGLKWMG

SEQ ID NO: 286 - protein, 32 aa, Artificial Sequence; identical to SEQ ID NO: 222
  Antibody RH1 (D) HFR3

SEQ ID NO: 287 - protein, 11 aa, Artificial Sequence
  Antibody RH1 (D) HFR4
WGQGTLVTVSA

SEQ ID NO: 288 - protein, 23 aa, Artificial Sequence
  Antibody RF1 LFR1
DVVMTQTPLSLPVSLGDQASMSC

SEQ ID NO: 289 - protein, 15 aa, Artificial Sequence
  Antibody RF1 LFR2
WYLQKPGQSPKLLIY

SEQ ID NO: 290 - protein, 32 aa, Artificial Sequence
  Antibody RF1 LFR3
GVPDRFSGSGSGTDFTLKISRVEAEDLGIYFC

SEQ ID NO: 291 - protein, 10 aa, Artificial Sequence
  Antibody RF1 LFR4
FGGGTKLEIK

SEQ ID NO: 292 - protein, 30 aa, Artificial Sequence
  Antibody RF1 HFR1
QVQLQQPGAELVKPGASVKMSCKASGYTFT

SEQ ID NO: 293 - protein, 14 aa, Artificial Sequence
  Antibody RF1 HFR2
WVMQRPGQGLEWIG

SEQ ID NO: 294 - protein, 32 aa, Artificial Sequence
  Antibody RF1 HFR3
KATLTVDTSSSTAFMQLSSLTSEDSAVYYCTT

SEQ ID NO: 295 - protein, 11 aa, Artificial Sequence
  Antibody RF1 HFR4
WGRGTTLTVSS

SEQ ID NO: 296 - protein, 23 aa, Artificial Sequence
  Antibody RF3 LFR1
DVVMTQTPLSLPVSLGDQASISC

SEQ ID NO: 297 - protein, 15 aa, Artificial Sequence
  Antibody RF3 LFR2
WYLQKPGQSPKLLIY

SEQ ID NO: 298 - protein, 32 aa, Artificial Sequence
  Antibody RF3 LFR3
GVPDRFGGSGSGTDFTLKISRVEAEDLGVYFC

SEQ ID NO: 299 - protein, 10 aa, Artificial Sequence
  Antibody RF3 LFR4
FGGGTKLEMK

SEQ ID NO: 300 - protein, 30 aa, Artificial Sequence
  Antibody RF3 HFR1
QVQLQQPGAELVRPGASVKLSCKASGYTFT

SEQ ID NO: 301 - protein, 14 aa, Artificial Sequence
  Antibody RF3 HFR2
WWKQRPGQGLEWIG

SEQ ID NO: 302 - protein, 32 aa, Artificial Sequence
  Antibody RF3 HFR3
KATLTVDISSTTAYMHLSSLTSEDSAVYYCAR

SEQ ID NO: 303 - protein, 11 aa, Artificial Sequence
  Antibody RF3 HFR4
WGQGTLVTVSA

SEQ ID NO: 304 - protein, 23 aa, Artificial Sequence
  Antibody RA5 (A) LFR1
DVLMTQTPLSLPVSLGDQASISC

SEQ ID NO: 305 - protein, 15 aa, Artificial Sequence
  Antibody RA5 (A) LFR2
WYLQKPGQSPKLLIY

SEQ ID NO: 306 - protein, 32 aa, Artificial Sequence
  Antibody RA5 (A) LFR3
GVPDRFSGSGSGTDFTLKISRVEAEDLGVYYC

SEQ ID NO: 307 - protein, 10 aa, Artificial Sequence
  Antibody RA5 (A) LFR4
FGGGTKLEIK

SEQ ID NO: 308 - protein, 30 aa, Artificial Sequence
  Antibody RA5 (A) HFR1
QVQLQQSGAELVRPGTSVKVSCKASGYAFT

SEQ ID NO: 309 - protein, 14 aa, Artificial Sequence
  Antibody RA5 (A) HFR2
WVKQRPGQGLEWIG

SEQ ID NO: 310 - protein, 32 aa, Artificial Sequence
  Antibody RA5 (A) HFR3
KATLTADKSSSTAYMQLSSLTSDDSAVYFCAR

SEQ ID NO: 311 - protein, 11 aa, Artificial Sequence
  Antibody RA5 (A) HFR4
WGQGTLVTVSA

SEQ ID NO: 312 - protein, 23 aa, Artificial Sequence
  Antibody RA5 (B) LFR1
DIVLTQSPATLSVTPGDSVSLSC

SEQ ID NO: 313 - protein, 15 aa, Artificial Sequence
  Antibody RA5 (B) LFR2
WYQQKSHESPRLLIK

SEQ ID NO: 314 - protein, 32 aa, Artificial Sequence
  Antibody RA5 (B) LFR3
GIPSRFSGSGSGTDFTLSINSVETEDFGMYFC

SEQ ID NO: 315 - protein, 10 aa, Artificial Sequence
  Antibody RA5 (B) LFR4
FGAGTKLELK

SEQ ID NO: 316 - protein, 30 aa, Artificial Sequence; identical to SEQ ID NO: 308
  Antibody RA5 (B) HFR1

SEQ ID NO: 317 - protein, 14 aa, Artificial Sequence
  Antibody RA5 (B) HFR2
WVKQRPGQGLEWIG

SEQ ID NO: 318 - protein, 32 aa, Artificial Sequence; identical to SEQ ID NO: 310
  Antibody RA5 (B) HFR3

SEQ ID NO: 319 - protein, 11 aa, Artificial Sequence
  Antibody RA5 (B) HFR4
WGQGTLVTVSA

SEQ ID NO: 320 - protein, 23 aa, Artificial Sequence
  Antibody RD3 LFR1
DVLMTQTPLSLPVSLGDQASISC

SEQ ID NO: 321 - protein, 15 aa, Artificial Sequence
  Antibody RD3 LFR2
WYLQKPGQSPKLLIY

SEQ ID NO: 322 - protein, 32 aa, Artificial Sequence; identical to SEQ ID NO: 306
  Antibody RD3 LFR3

SEQ ID NO: 323 - protein, 10 aa, Artificial Sequence
  Antibody RD3 LFR4
FGGGTKLEIK

SEQ ID NO: 324 - protein, 30 aa, Artificial Sequence
  Antibody RD3 HFR1
QVQLQQPGAELVKPGASVKLSCKASGYTFT

SEQ ID NO: 325 - protein, 14 aa, Artificial Sequence
  Antibody RD3 HFR2
WVKQRPGQGLEWIG

SEQ ID NO: 326 - protein, 32 aa, Artificial Sequence
  Antibody RD3 HFR3
KATLTVDKSSSTAYMQLSSLTSEDSAVYYCAN

SEQ ID NO: 327 - protein, 11 aa, Artificial Sequence
  Antibody RD3 HFR4
WGQGTTLTVSS

SEQ ID NO: 328 - protein, 23 aa, Artificial Sequence
  Antibody RC1 LFR1
DVVMTQTPLSLPVSLGDQASISC

SEQ ID NO: 329 - protein, 15 aa, Artificial Sequence
  Antibody RC1 LFR2
WYQQKPGQSPKLLIF

SEQ ID NO: 330 - protein, 32 aa, Artificial Sequence; identical to SEQ ID NO: 242
  Antibody RC1 LFR3

SEQ ID NO: 331 - protein, 10 aa, Artificial Sequence
  Antibody RC1 LFR4
FGSGTKLEIK

SEQ ID NO: 332 - protein, 30 aa, Artificial Sequence
  Antibody RC1 HFR1
EVQLQQSGAELVKPGASVKLSCTASGLNIK

SEQ ID NO: 333 - protein, 14 aa, Artificial Sequence
  Antibody RC1 HFR2
WVKQRPEQGLEWIG

SEQ ID NO: 334 - protein, 32 aa, Artificial Sequence
  Antibody RC1 HFR3
KATITADTSSNTAYLHLSSLTSEDTAVYYCTN

SEQ ID NO: 335 - protein, 11 aa, Artificial Sequence
  Antibody RC1 HFR4
WGQGTLVTVSA

SEQ ID NO: 336 - protein, 23 aa, Artificial Sequence
  Antibody RF2 LFR1
DIVMTQTPLSLPVSLGDQASISC

SEQ ID NO: 337 - protein, 15 aa, Artificial Sequence
  Antibody RF2 LFR2
WYLQKPGQSPKLLIY

SEQ ID NO: 338 - protein, 32 aa, Artificial Sequence; identical to SEQ ID NO: 242
  Antibody RF2 LFR3

SEQ ID NO: 339 - protein, 10 aa, Artificial Sequence
  Antibody RF2 LFR4
FGAGTKLELK

SEQ ID NO: 340 - protein, 30 aa, Artificial Sequence
  Antibody RF2 HFR1
EVQLQQSGAELVRPGALVKLSSKASGFNIK

SEQ ID NO: 341 - protein, 14 aa, Artificial Sequence
  Antibody RF2 HFR2
WVKQRPEQGLEWIG

SEQ ID NO: 342 - protein, 32 aa, Artificial Sequence
  Antibody RF2 HFR3
KATITADTSSNTTYLQLSSLTSEDTAVYYCTR

SEQ ID NO: 343 - protein, 11 aa, Artificial Sequence
  Antibody RF2 HFR4
WGQGTSVTVSS